(19) United States
(12) Patent Application Publication
(10) Pub. No.: US 2018/0312913 A1
(43) Pub. Date: Nov. 1, 2018

(54) METHODS AND SYSTEMS FOR NUCLEIC ACID AMPLIFICATION

(71) Applicant: Coyote Bioscience Co., Ltd., Beijing (CN)

(72) Inventors: Xiang Li, Beijing (CN); Yumei Wei, Beijing (CN); Huiying Feng, Xicheng District (CN); Yuning Wang, Beijing (CN)

(21) Appl. No.: 15/953,304

(22) Filed: Apr. 13, 2018

Related U.S. Application Data
(63) Continuation of application No. PCT/CN2016/107443, filed on Nov. 28, 2016.
Application No. PCT/CN2016/107443 is a continuation-in-part of application No. PCT/CN2015/095763, filed on Nov. 27, 2015.

(51) Int. Cl. C12Q 1/6844; C12Q 1/686; C12Q 1/70

(52) U.S. Cl. CPC C12Q 1/6846; C12Q 1/70; C12Q 1/686

(57) ABSTRACT

The present disclosure provides methods and systems for amplifying and analyzing nucleic acid samples.

### CROSS-REFERENCE

This application is a continuation of PCT Application Serial No. PCT/CN2016/107443, filed Nov. 28, 2016, which is a continuation-in-part of PCT Application Serial No. PCT/CN2015/095763, filed Nov. 27, 2015, which applications are entirely incorporated herein by reference.

### SEQUENCE LISTING

The instant application contains a Sequence Listing which has been filed electronically in ASCII format and is hereby incorporated by reference in its entirety. Said ASCII copy, created on Jul. 5, 2018, is named 45769725301SL.txt and is 2,359 bytes in size.

### BACKGROUND

Nucleic acid amplification methods permit selected amplification and identification of nucleic acids of interest from a complex mixture, such as a biological sample. To detect a nucleic acid in a biological sample, the biological sample is typically processed to isolate nucleic acids from other components of the biological sample and other agents that may interfere with the nucleic acid and/or amplification. Following isolation of the nucleic acid of interest from the biological sample, the nucleic acid of interest can be amplified, via, for example, amplification methods, such as thermal cycling based approaches (e.g., polymerase chain reaction (PCR)). Following amplification of the nucleic acid of interest, the products of amplification can be detected and the results of detection interpreted by an end-user. The extraction of nucleic acid from a biological sample prior to amplification of the nucleic acid, however, can be time consuming, resulting in a reduced time efficiency for the process as a whole.

Point-of-care (POC) testing has the potential to improve the detection and management of infections (e.g., infectious diseases, contamination of food, contamination of soil, etc.) in resource-limited settings with poor laboratory infrastructure, or in remote areas where there are delays in the receipt of laboratory results and potential complications to following up with patients. POC testing also may render state of the art health care facilities more capable of delivering sample-to-answer results to patients during a single visit. Inefficiencies in POC methods and devices, however, limit what can be achieved. For example, preparation of nucleic acids (e.g., of a pathogen) from complex sample types (e.g., biological samples) entails highly skilled personnel, in a dedicated laboratory space, to manually perform multiple processing steps and subsequent testing, with reporting of results often occurring hours or even days later.

Thus, there exists a need for rapid, accurate methods and devices for analyzing nucleic acids from complex sample types. Such methods and devices may be useful, for example, in realizing fast sample-to-answer detection and management of diseases detectable via their nucleic acid.

### SUMMARY

The present disclosure provides methods and systems for efficient amplification of nucleic acids, such as RNA and DNA molecules. Amplified nucleic acid product can be detected rapidly and with good sensitivity.

In one aspect, the present disclosure provides a method for detecting a target nucleic acid molecule in a biological sample. The method may comprise: (a) mixing the biological sample with a lysis buffer to obtain a mixture; (b) incubating the mixture at a temperature from about 15° C. to 70° C. for a period of time of no more than about 15 minutes; (c) adding the mixture from (b) to a reaction vessel comprising reagents necessary for conducting nucleic acid amplification, the reagents comprising (i) a deoxyribonucleic acid (DNA) polymerase, and (ii) a primer set for the target nucleic acid molecule, to obtain a reaction mixture; and (d) subjecting the reaction mixture in the reaction vessel to multiple cycles of a primer extension reaction to generate amplified product(s) that is indicative of a presence of the target nucleic acid molecule, each cycle comprising (i) incubating the reaction mixture at a denaturing temperature for a denaturing duration that is less than or equal to 60 seconds, followed by (ii) incubating the reaction mixture at an elongation temperature for an elongation duration that is less than or equal to 60 seconds, thereby amplifying the target nucleic acid molecule.

In one aspect, the present disclosure provides a method for detecting a target nucleic acid molecule in a biological sample. The method may comprise: (a) mixing the biological sample with a lysis buffer to obtain a mixture; (b) incubating the mixture at a temperature from about 15° C. to 70° C. for a period of time of no more than about 15 minutes; (c) adding the mixture from (b) to a reaction vessel comprising reagents necessary for conducting nucleic acid amplification, the reagents comprising (i) a deoxyribonucleic acid (DNA) polymerase and optionally a reverse transcriptase, and (ii) a primer set for the target nucleic acid molecule, to obtain a reaction mixture; and (d) subjecting the reaction mixture in the reaction vessel to a plurality of series of primer extension reactions to generate amplified product(s) that is indicative of a presence of the target nucleic acid molecule in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In some embodiments, the method further comprises, prior to (a), suspending the biological sample in solution to obtain a homogenized preparation comprising the biological sample.

In some embodiments, the method further comprises, prior to (a), subjecting the biological sample to centrifugation to yield a solution comprising the biological sample and a pellet. In some embodiments, the method further comprises, prior to (a), subjecting the biological sample to centrifugation to yield a solution and a pellet comprising the biological sample.

In some embodiments, the method further comprises, between (b) and (c), subjecting the mixture to centrifugation to yield a supernatant comprising the biological sample.

In some embodiments, the biological sample is obtained directly from a source thereof without pre-culturing, non-selective enrichment, selective enrichment, plating on differential medium, and/or presumptive biomedical identification.

The biological sample may be from a tissue or fluid of a subject. In some embodiments, the tissue or fluid is stool. The biological sample may be obtained by an oral or rectal swab.

In some embodiments, the biological sample includes a soil or food sample. The food sample may be a dairy sample. For example, the dairy sample may include milk.

In some embodiments, the mixture is added to the reaction vessel in (c) without undergoing DNA or ribonucleic acid (RNA) extraction. In some embodiments, the mixture is added to the reaction vessel in (c) without undergoing purification. In some embodiments, the mixture is added to the reaction vessel in (c) without undergoing DNA or ribonucleic acid (RNA) concentration.

In some embodiments, the temperature in (b) is from about 20° C. to 40° C. In some embodiments, the period of time in (b) is no more than about 10 minutes. For example, the period of time in (b) is no more than about 1 minute.

In some embodiments, the biological sample is not treated with a detergent.

In some embodiments, the lysis buffer comprises NaOH. The lysis buffer may have a pH from about 8 to 13.

In some embodiments, in (a), a ratio of the biological sample to the lysis buffer is between about 1:1 (wt/vol) to about 1:10 (wt/vol).

In some embodiments, the reagents in (c) include reagents necessary for conducting reverse transcription amplification and deoxyribonucleic acid (DNA) amplification. For example, the reagents may comprise a reverse transcriptase.

In some embodiments, the reagents in (c) comprise a reporter agent that yields a detectable signal indicative of a presence of the amplified product(s). For example, an intensity of the detectable signal may be proportional to an amount of the amplified product(s) or target nucleic acid molecule. For example, the reporter agent may be a dye.

The target nucleic acid molecule may be a DNA and/or a ribonucleic acid (RNA). In some embodiments, the RNA is viral RNA.

In some embodiments, the denaturing temperature is from about 90° C. to 100° C. For example, the denaturing temperature may be from about 92° C. to 95° C. In some embodiments, the elongation temperature is from about 35° C. to 72° C. For example, the elongation temperature may be from about 45° C. to 65° C. The denaturing duration may be less than or equal to about 30 seconds. The elongation duration may be less than or equal to about 30 seconds.

In some embodiments, the amplifying yields a detectable amount of the amplified product(s) indicative of a presence of the target nucleic acid molecule in the biological sample at a cycle threshold value (Ct) of less than 30. In some embodiments, the amplifying yields a detectable amount of the amplified product(s) indicative of a presence of the target nucleic acid molecule in the sample at a time period of 10 minutes or less.

In some embodiments, the method further comprises detecting an amount of the amplified product(s).

In some embodiments, the method further comprises outputting information indicative of an amount of the amplified product(s) to a recipient. The recipient may be a treating physician, a pharmaceutical company, or the subject. The information may be outputted as a report.

In some embodiments, the operation (d) is conducted in 35 cycles or less.

In some embodiments, the target nucleic acid molecule is associated with a disease. The disease may be associated with a virus. The virus may be an RNA virus or a DNA virus. For example, the virus may be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, Varicella virus, enterovirus, and norovirus. The influenza virus may be selected from the group consisting of H1N1 virus, H3N2 virus, H7N9 virus and H5N1 virus. The adenovirus may be adenovirus type 55 (ADV55) or adenovirus type 7 (ADV7). The hepatitis C virus may be armored RNA-hepatitis C virus (RNA-HCV). The enterovirus may be a Coxsackie virus. The Coxsackie virus may be Coxsackie virus A16. The enterovirus may be enterovirus 71. The norovirus may be norovirus GI or norovirus GII.

In some embodiments, the disease is associated with a pathogenic bacterium or a pathogenic protozoan. The pathogenic bacterium may be a gram-positive or gram-negative pathogenic bacterium. The pathogenic bacterium may be selected from the group consisting of Staphylococcus aureus, Listeria monocytogenes, Escherichia coli, Enterobacter sakazakii, Vibrio Parahemolyticus, and Shigella spp. For example, the pathogenic bacterium may be Mycobacterium tuberculosis. The pathogenic protozoan may be Plasmodium. In some embodiments, the pathogenic bacterium is Salmonella.

In some embodiments, the amplified product in (d) is amplified DNA product.

In some embodiments, the method further comprises subjecting the target nucleic acid molecule to one or more denaturing conditions prior to (d). The one or more denaturing conditions may be selected from a denaturing temperature profile and a denaturing agent.

In some embodiments, the method further comprises subjecting the target nucleic acid molecule to one or more denaturing conditions between a first series and a second series of the plurality of series of primer extension reactions. The individual series may differ with respect to at least any one of ramping rate between denaturing temperature and elongation temperature, denaturing temperature, denaturing duration, elongation temperature and elongation duration. In some embodiments, the individual series differ with respect to at least any two of ramping rate between denaturing temperature and elongation temperature, denaturing temperature, denaturing duration, elongation temperature and elongation duration.

In some embodiments, the plurality of series of primer extension reactions comprises a first series and a second series, the first series comprising more than ten cycles, each cycle of the first series comprising (i) incubating the reaction mixture at about 92° C.-95° C. for no more than 30 seconds, followed by (ii) incubating the reaction mixture at about 35° C.-65° C. for no more than 1 minute, the second series comprising more than ten cycles, each cycle of the second series comprising (i) incubating the reaction mixture at about 92° C.-95° C. for no more than 30 seconds, followed by (ii) incubating the reaction mixture at about 40° C.-60° C. for no more than 1 minute.

In some embodiments, the plurality of series of primer extension reactions yields a detectable amount of amplified product that is indicative of a presence of the target nucleic acid in the biological sample with a lower cycle threshold value as compared to a single series of primer extension reactions under comparable denaturing and elongation conditions.

In some embodiments, the method further comprises, prior to (d), pre-heating the biological sample at a pre-heating temperature from 90° C. to 100° C. for a pre-heating duration of no more than 10 minutes. For example, the pre-heating duration may be no more than 1 minute.

In one aspect, the present disclosure provides a system for detecting a target nucleic acid molecule in a biological sample. The system may comprise: an input unit that receives a request from a user to process the biological sample to detect the target nucleic acid molecule; and one or more computer processors operatively coupled to the input unit. The one or more computer processors may be individually or collectively programmed to: (a) mix the biological sample with a lysis buffer to obtain a mixture; (b) incubate the mixture at a temperature from about 15° C. to 70° C. for a period of time of no more than about 15 minutes; (c) add the mixture from (b) to a reaction vessel comprising reagents necessary for conducting nucleic acid amplification, the reagents comprising (i) a deoxyribonucleic acid (DNA) polymerase, and (ii) a primer set for the target nucleic acid molecule, to obtain a reaction mixture; and (d) subject the reaction mixture in the reaction vessel to multiple cycles of a primer extension reaction to generate amplified product(s) that is indicative of a presence of the target nucleic acid molecule, each cycle comprising (i) incubating the reaction mixture at a denaturing temperature for a denaturing duration that is less than or equal to 60 seconds, followed by (ii) incubating the reaction mixture at an elongation temperature for an elongation duration that is less than or equal to 60 seconds, thereby amplifying the target nucleic acid molecule.

In one aspect, the present disclosure provides a system for detecting a target nucleic acid molecule in a biological sample. The system may comprise: an input unit that receives a request from a user to process the biological sample to detect the target nucleic acid molecule; and one or more computer processors operatively coupled to the input unit. The one or more computer processors may be individually or collectively programmed to: (a) mix the biological sample with a lysis buffer to obtain a mixture; (b) incubate the mixture at a temperature from about 15° C. to 70° C. at a period of time of no more than about 15 minutes; (c) add the mixture from (b) to a reaction vessel comprising reagents necessary for conducting nucleic acid amplification, the reagents comprising (i) a deoxyribonucleic acid (DNA) polymerase and optionally a reverse transcriptase, and (ii) a primer set for the target nucleic acid molecule, to obtain a reaction mixture; and (d) subject the reaction mixture in the reaction vessel to a plurality of series of primer extension reactions to generate amplified product(s) that is indicative of a presence of the target nucleic acid molecule in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In some embodiments, the system further comprises a biological sample treatment module that mixes the biological sample with the lysis buffer to obtain the mixture. The biological sample treatment module may incubate the mixture.

In some embodiments, the system further comprises an amplification module operatively coupled to the biological sample treatment module. The amplification module may (i) add an amount of the mixture from the biological sample treatment module to the reaction vessel and (ii) subject the reaction mixture in the reaction vessel to the primer extension reaction(s) to generate the amplified product that is indicative of a presence of the target nucleic acid molecule.

In some embodiments, the system further comprises an output module operatively coupled to the one or more computer processors. The output module may output information regarding the target nucleic acid molecule or the amplified DNA product to a recipient.

The one or more computer processors may be individually or collectively programmed to, prior to (a), suspend the biological sample in solution to obtain a homogenized preparation comprising the biological sample.

In some embodiments, the one or more computer processors are individually or collectively programmed to, prior to (a), subject the biological sample to centrifugation to yield a solution comprising the biological sample and a pellet. In some embodiments, the one or more computer processors are individually or collectively programmed to, prior to (a), subject the biological sample to centrifugation to yield a solution and a pellet comprising the biological sample.

In some embodiments, the one or more computer processors are individually or collectively programmed to, between (b) and (c), subject the mixture to centrifugation to yield a supernatant comprising the biological sample.

The biological sample may be obtained directly from a source thereof without pre-culturing, non-selective enrichment, selective enrichment, plating on differential medium, and/or presumptive biomedical identification.

In some embodiments, the biological sample is from a tissue or fluid of a subject. The tissue or fluid may be stool. The biological sample may be obtained by an oral or rectal swab.

In some embodiments, the biological sample includes a soil or food sample. The food sample may be a dairy sample. In some embodiments, the dairy sample includes milk.

In some embodiments, the mixture is added to the reaction vessel in (c) without undergoing DNA or ribonucleic acid (RNA) extraction. In some embodiments, the mixture is added to the reaction vessel in (c) without undergoing purification. In some embodiments, the mixture is added to the reaction vessel in (c) without undergoing DNA or ribonucleic acid (RNA) concentration.

The temperature in (b) may be from about 20° C. to 40° C.

The period of time in (b) may be no more than about 10 minutes. In some embodiments, the period of time in (b) is no more than about 1 minute.

In some embodiments, the biological sample is not treated with a detergent.

In some embodiments, the lysis buffer comprises NaOH. The lysis buffer may have a pH from about 8 to 13.

In some embodiments, in (a), a ratio of the biological sample to the lysis buffer is between about 1:1 (wt/vol) to about 1:10 (wt/vol).

The reagents in (c) may include reagents necessary for conducting reverse transcription amplification and deoxyribonucleic acid (DNA) amplification. For example, the reagents may comprise a reverse transcriptase.

In some embodiments, the reagents in (c) comprise a reporter agent that yields a detectable signal indicative of a presence of the amplified product(s). Intensity of the detectable signal may be proportional to an amount of the amplified product(s) or target nucleic acid molecule. The reporter agent may be a dye.

The target nucleic acid molecule may be a DNA. In some embodiments, the target nucleic acid molecule is ribonucleic acid (RNA). The RNA may be viral RNA.

The denaturing temperature may be from about 90° C. to 100° C. For example, the denaturing temperature may be from about 92° C. to 95° C.

The elongation temperature may be from about 35° C. to 72° C. For example, the elongation temperature may be from about 45° C. to 65° C.

The denaturing duration may be less than or equal to about 30 seconds. The elongation duration may be less than or equal to about 30 seconds.

In some embodiments, the amplifying yields a detectable amount of the amplified product(s) indicative of a presence of the target nucleic acid molecule in the biological sample at a cycle threshold value (Ct) of less than 30.

In some embodiments, the amplifying yields a detectable amount of the amplified product(s) indicative of a presence of the target nucleic acid molecule in the sample at a time period of 10 minutes or less.

In some embodiments, the one or more computer processors are individually or collectively programmed to detect an amount of the amplified product(s).

In some embodiments, the one or more computer processors are individually or collectively programmed to output information indicative of an amount of the amplified product(s) to a recipient. The recipient may be a treating physician, a pharmaceutical company, or the subject. The information may be outputted as a report.

In some embodiments, (d) is conducted in 35 cycles or less.

The target nucleic acid molecule may be associated with a disease.

In some embodiments, the disease is associated with a virus. The virus may be an RNA virus or a DNA virus. For example, the virus may be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, Varicella virus, enterovirus, and norovirus. The influenza virus may be selected from the group consisting of H1N1 virus, H3N2 virus, H7N9 virus and H5N1 virus. The adenovirus may be adenovirus type 55 (ADV55) or adenovirus type 7 (ADV7). The hepatitis C virus may be armored RNA-hepatitis C virus (RNA-HCV). The enterovirus may be a Coxsackie virus. The Coxsackie virus may be Coxsackie virus A16. The norovirus may be norovirus GI or norovirus GII.

In some embodiments, the disease is associated with a pathogenic bacterium or a pathogenic protozoan. The pathogenic bacterium may be a gram-positive or gram-negative pathogenic bacterium. The pathogenic bacterium may be selected from the group consisting of Staphylococcus aureus, Listeria monocytogenes, Escherichia coli, Enterobacter sakazakii, Vibrio Parahemolyticus, and Shigella spp. In some embodiments, the pathogenic bacterium is Mycobacterium tuberculosis. In some embodiments, the pathogenic bacterium is Salmonella. The pathogenic protozoan may be Plasmodium.

In some embodiments, the amplified product in (d) is amplified DNA product.

In some embodiments, the one or more computer processors are individually or collectively programmed to subject the target nucleic acid molecule to one or more denaturing conditions prior to (d). The one or more denaturing conditions may be selected from a denaturing temperature profile and a denaturing agent.

In some embodiments, the one or more computer processors are individually or collectively programmed to subject the target nucleic acid molecule to one or more denaturing conditions between a first series and a second series of the plurality of series of primer extension reactions. The individual series may differ with respect to at least any one of ramping rate between denaturing temperature and elongation temperature, denaturing temperature, denaturing duration, elongation temperature and elongation duration. In some embodiments, the individual series differ with respect to at least any two of ramping rate between denaturing temperature and elongation temperature, denaturing temperature, denaturing duration, elongation temperature and elongation duration.

In some embodiments, the plurality of series of primer extension reactions comprises a first series and a second series, the first series comprising more than ten cycles, each cycle of the first series comprising (i) incubating the reaction mixture at about 92° C.-95° C. for no more than 30 seconds, followed by (ii) incubating the reaction mixture at about 35° C.-65° C. for no more than 1 minute, the second series comprising more than ten cycles, each cycle of the second series comprising (i) incubating the reaction mixture at about 92° C.-95° C. for no more than 30 seconds, followed by (ii) incubating the reaction mixture at about 40° C.-60° C. for no more than 1 minute.

In some embodiments, the plurality of series of primer extension reactions yields a detectable amount of amplified product that is indicative of a presence of the target nucleic acid in the biological sample with a lower cycle threshold value as compared to a single series of primer extension reactions under comparable denaturing and elongation conditions.

In some embodiments, the one or more computer processors are individually or collectively programmed to, prior to (d), pre-heat the biological sample at a pre-heating temperature from 90° C. to 100° C. for a pre-heating duration of no more than 10 minutes. The pre-heating duration may be no more than 1 minute.

In one aspect, the present disclosure provides a computer readable medium comprising machine executable code that, upon execution by one or more computer processors, implements a method of detecting a target nucleic acid molecule in a biological sample. The method may comprise: (a) mixing the biological sample with a lysis buffer to obtain a mixture; (b) incubating the mixture at a temperature from about 15° C. to 70° C. for a period of time of no more than about 15 minutes; (c) adding the mixture from (b) to a reaction vessel comprising reagents necessary for conducting nucleic acid amplification, the reagents comprising (i) a deoxyribonucleic acid (DNA) polymerase, and (ii) a primer set for the target nucleic acid molecule, to obtain a reaction mixture; and (d) subjecting the reaction mixture in the reaction vessel to multiple cycles of a primer extension reaction to generate amplified product(s) that is indicative of a presence of the target nucleic acid molecule, each cycle comprising (i) incubating the reaction mixture at a denaturing temperature for a denaturing duration that is less than or equal to 60 seconds, followed by (ii) incubating the reaction mixture at an elongation temperature for an elongation duration that is less than or equal to 60 seconds, thereby amplifying the target nucleic acid molecule.

In one aspect, the present disclosure provides a computer readable medium comprising machine executable code that, upon execution by one or more computer processors, implements a method of detecting a target nucleic acid molecule in a biological sample. The method may comprise: (a) mixing the biological sample with a lysis buffer to obtain a mixture; (b) incubating the mixture at a temperature from about 15° C. to 70° C. at a period of time of no more than about 15 minutes; (c) adding the mixture from (b) to a reaction vessel comprising reagents necessary for conducting nucleic acid amplification, the reagents comprising (i) a deoxyribonucleic acid (DNA) polymerase and optionally a reverse transcriptase, and (ii) a primer set for the target nucleic acid molecule, to obtain a reaction mixture; and (d) subjecting the reaction mixture in the reaction vessel to a plurality of series of primer extension reactions to generate amplified product(s) that is indicative of a presence of the target nucleic acid molecule in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In one aspect, the present disclosure provides a method for detecting a target nucleic acid molecule in a biological sample. The method may comprise: (a) mixing the biological sample with a lysis buffer to obtain a mixture, wherein the biological sample includes a stool sample or milk sample; (b) incubating the mixture at an incubation temperature for an incubation time period; (c) adding the mixture from (b) to a reaction vessel comprising reagents necessary for conducting nucleic acid amplification, the reagents comprising (i) a deoxyribonucleic acid (DNA) polymerase, and (ii) a primer set for the target nucleic acid molecule, to obtain a reaction mixture; and (d) subjecting the reaction mixture in the reaction vessel to multiple cycles of a primer extension reaction to generate amplified product(s) that is indicative of a presence of the target nucleic acid molecule, each cycle comprising (i) incubating the reaction mixture at a denaturing temperature for a denaturing duration that is less than or equal to 60 seconds, followed by (ii) incubating the reaction mixture at an elongation temperature for an elongation duration that is less than or equal to 60 seconds, thereby amplifying the target nucleic acid molecule.

In one aspect, the present disclosure provides a method for detecting a target nucleic acid molecule in a biological sample. The method may comprise: (a) mixing the biological sample with a lysis buffer to obtain a mixture, wherein the biological sample includes a stool sample or milk sample; (b) incubating the mixture at an incubation temperature for an incubation time period; (c) adding the mixture from (b) to a reaction vessel comprising reagents necessary for conducting nucleic acid amplification, the reagents comprising (i) a deoxyribonucleic acid (DNA) polymerase and optionally a reverse transcriptase, and (ii) a primer set for the target nucleic acid molecule, to obtain a reaction mixture; and (d) subjecting the reaction mixture in the reaction vessel to a plurality of series of primer extension reactions to generate amplified product(s) that is indicative of a presence of the target nucleic acid molecule in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In another aspect, the present disclosure provides a method for detecting Salmonella in a stool sample. The method may comprise: (a) mixing the stool sample with a lysis buffer to obtain a mixture; (b) incubating the mixture at an incubation temperature greater than 15° C. for an incubation time period of no more than about 15 minutes; (c) adding the mixture from (b) to a reaction vessel comprising reagents necessary for conducting nucleic acid amplification, the reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof, to obtain a reaction mixture; and (d) subjecting the reaction mixture in the reaction vessel to a plurality of series of primer extension reactions to generate amplified product(s) that is indicative of a presence of the target nucleic acid(s) in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In some embodiments, the lysis buffer is alkaline.

In some embodiments, the lysis buffer has a pH from about 8 to 13.

In some embodiments, the method further comprises, between (b) and (c), subjecting the mixture to centrifugation to yield a supernatant for use as the mixture in subsequent steps.

In some embodiments, the stool sample has been cultivated for microbial proliferation.

In some embodiments, the cultivation for microbial proliferation comprises subjecting the stool sample under enrichment culturing conditions for a cultivation period of time.

In some embodiments, the stool sample is obtained directly from a source thereof without pre-culturing, non-selective enrichment, selective enrichment, plating on differential medium, and/or presumptive biomedical identification.

In some embodiments, the stool sample is a solid stool sample.

In some embodiments, the stool sample is a liquid stool sample.

In some embodiments, the liquid stool sample is a watery diarrhea stool.

In some embodiments, the stool sample is obtained by a swab.

In some embodiments, the mixture is added to the reaction vessel in (c) without undergoing DNA or ribonucleic acid (RNA) extraction.

In some embodiments, the mixture is added to the reaction vessel in (c) without undergoing purification.

In some embodiments, the mixture is added to the reaction vessel in (c) without undergoing DNA or ribonucleic acid (RNA) concentration.

In some embodiments, the temperature in (b) is from about 80° C. to 100° C.

In some embodiments, the incubation time period in (b) is no less than about 2 minutes.

In some embodiments, the incubation time period in (b) is about 10 minute.

In some embodiments, the stool sample is not treated with a detergent.

In some embodiments, the method further comprises, prior to (a), adding a suspension buffer to the stool sample to obtain a homogenized preparation of the stool sample.

In some embodiments, the suspension buffer comprises NaCl, PBS, and/or HEPES.

In some embodiments, a ratio of the stool sample to the suspension buffer is between about 1:1 (wt/vol) to about 1:100 (wt/vol).

In some embodiments, in (a), a ratio of the homogenized preparation of the stool sample to the lysis buffer is between about 5:1 (vol/vol) to about 1:5 (vol/vol).

In some embodiments, the reagents in (c) include reagents necessary for conducting reverse transcription amplification and deoxyribonucleic acid (DNA) amplification.

In some embodiments, the reagents comprise a reverse transcriptase.

In some embodiments, the reagents in (c) comprise reporter agent that yields a detectable signal indicative of a presence of the amplified product.

In some embodiments, an intensity of the detectable signal is proportional to an amount of the amplified product or target nucleic acid.

In some embodiments, the reporter agent is a sequence-specific oligonucleotide probe that is optically active when hybridized with the amplified product.

In some embodiments, the sequence-specific oligonucleotide probe links to optically-active reporter agent and optionally, quencher.

In some embodiments, the reporter agent is a sequence-specific oligonucleotide probe that has blocked optical activity when hybridized with the amplified product.

In some embodiments, the oligonucleotide probe is optically active upon breakdown.

In some embodiments, the reporter agent is a dye.

In some embodiments, the sequence-specific oligonucleotide probe hybridizes with a region on the target nucleic acid between the primers in the primer set capable of specifically binding to the target nucleic acid.

In some embodiments, the primer sets include both a primer set capable of specifically binding to a target nucleic acid sequence from a Salmonella genome or variants thereof and a primer set capable of specifically binding to a target nucleic acid sequence from a Salmonella transcriptome, or variants thereof.

In some embodiments, each target nucleic acid is independently a DNA or an RNA.

In some embodiments, the RNA is an mRNA.

In some embodiments, the denaturing temperature is from about 90° C. to 100° C.

In some embodiments, the elongation temperature is from about 35° C. to 72° C.

In some embodiments, the denaturing duration is less than or equal to about 30 seconds.

In some embodiments, the elongation duration is less than or equal to about 30 seconds.

In some embodiments, the amplifying yields a detectable amount of the amplified product indicative of a presence of the target nucleic acid(s) in the stool sample at a cycle threshold value (Ct) of less than 30.

In some embodiments, the amplifying yields a detectable amount of the amplified product indicative of a presence of the target nucleic acid(s) in the stool sample at a time period of 30 minutes or less.

In some embodiments, the method further comprises detecting an amount and/or presence of the amplified product(s).

In some embodiments, the method further comprises outputting information indicative of an amount and/or presence of the amplified product(s) to a recipient.

In some embodiments, the information is outputted as a report.

In some embodiments, each series in (d) is conducted in 35 cycles or less.

In some embodiments, the amplified product in (d) is an amplified DNA product.

In some embodiments, the method further comprises subjecting the target nucleic acid to one or more denaturing conditions prior to (d).

In some embodiments, the one or more denaturing conditions are selected from a denaturing temperature profile and a denaturing agent.

In some embodiments, the method further comprises subjecting the target nucleic acid molecule to one or more denaturing conditions between any two consecutive series of the plurality of series of primer extension reactions.

In some embodiments, the individual series differ with respect to at least any one of ramping rate between denaturing temperature and elongation temperature, denaturing temperature, denaturing duration, elongation temperature and elongation duration.

In some embodiments, the individual series differ with respect to at least any two of ramping rate between denaturing temperature and elongation temperature, denaturing temperature, denaturing duration, elongation temperature and elongation duration.

In some embodiments, the plurality of series of primer extension reactions comprises a first series and a second series, the first series comprising ten or more cycles, each cycle of the first series comprising (i) incubating the reaction mixture at about 92° C.-95° C. for no more than 30 seconds, followed by (ii) incubating the reaction mixture at about 35° C.-65° C. for no more than 1 minute, the second series comprising ten or more cycles, each cycle of the second series comprising (i) incubating the reaction mixture at about 92° C.-95° C. for no more than 30 seconds, followed by (ii) incubating the reaction mixture at about 40° C.-60° C. for no more than 1 minute.

In some embodiments, the method further comprises incubating the reaction mixture at about 92° C.-95° C. for no more than 120 seconds between the first series and the second series.

In some embodiments, the plurality of series of primer extension reactions yields a detectable amount of amplified product that is indicative of a presence of the target nucleic acid in the stool sample with a lower cycle threshold value as compared to one series of primer extension reactions under comparable denaturing and elongation conditions.

In some embodiments, the method further comprises, prior to (d), pre-heating the stool sample at a pre-heating temperature from 90° C. to 100° C. for a pre-heating duration of no more than 10 minutes.

In some embodiments, the pre-heating duration is no more than 1 minute.

In some embodiments, the target nucleic acid is invA mRNA.

In some embodiments, the primer set includes a forward primer as depicted in SEQ ID NO: 1 (TGCTCGTTTACGACCTGAATTA) and a reverse primer depicted in SEQ ID NO: 2 (ACACCAATATCGCCAGTACG).

In some embodiments, the sequence-specific oligonucleotide probe comprise a nucleic acid sequence as depicted in SEQ ID NO: 3 (TCTGGTTGATTTCCTGATCGCACTGA).

In some embodiments, the primer set includes a forward primer as depicted in SEQ ID NO: 4 (TCGTTTACGACCTGAATTAC) and a reverse primer depicted in SEQ ID NO: 5 (TAGAACGACCCCATAAACA).

In some embodiments, the sequence-specific oligonucleotide probe comprise a nucleic acid sequence as depicted in SEQ ID NO: 6 (CTGGTTGATTTCCTGATCGCACT).

In some embodiments, the target nucleic acid is ttr gene.

In some embodiments, the primer set includes a forward primer as depicted in SEQ ID NO: 7 (CTCACCAGGAGATTACAACATGG) and a reverse primer depicted in SEQ ID NO: 8 (AGCTCAGACCAAAAGTGACCATC).

In some embodiments, the sequence-specific oligonucleotide probe comprise a nucleic acid sequence as depicted in SEQ ID NO: 9 (CACCGACGGCGAGACCGACTTT).

In some embodiments, the reagents further comprise MgCl₂.

In some embodiments, the reagents further comprise about 1.5 mM MgCl₂.

In some embodiments, the reagents further comprise about 0.1 to 0.5 mM dNTP(s).

In some embodiments, the reagents comprise about 0.1-1.0 μM forward primer(s) and reverse primer(s).

In some embodiments, the reagents comprise about 0.1-0.5 μM sequence-specific oligonucleotide probe(s).

In another aspect, the present disclosure provides use of an agent for the manufacture of a kit for detecting Salmonella in a stool sample. The detecting may comprise: (a) mixing the stool sample with a lysis buffer to obtain a mixture; (b) incubating the mixture at an incubation temperature greater than 15° C. for an incubation time period of no more than about 15 minutes; (c) adding the mixture from (b) to a reaction vessel comprising reagents necessary for conducting nucleic acid amplification, the reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof, to obtain a reaction mixture; and (d) subjecting the reaction mixture in the reaction vessel to a plurality of series of primer extension reactions to generate amplified product(s) that is indicative of a presence of the target nucleic acid(s) in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition. The agent may be the primer set(s).

In another aspect, the present disclosure provides a computer-assisted method for detecting Salmonella in a stool sample. The method may comprise: (a) an input step for receiving a request from a user to process the stool sample to detect Salmonella in the stool sample; (b) a mixing step for mixing the stool sample with a lysis buffer to obtain a mixture; (c) an incubating step for incubating the mixture at an incubation temperature greater than 15° C. for an incubation time period of no more than about 15 minutes; (d) an adding step for adding the mixture from (c) to a reaction vessel comprising reagents necessary for conducting nucleic acid amplification, the reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof, to obtain a reaction mixture; and (e) a reacting step for subjecting the reaction mixture in the reaction vessel to a plurality of series of primer extension reactions to generate amplified product(s) that is indicative of a presence of the target nucleic acid(s) in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In another aspect, the present disclosure provides a computer-assisted system for detecting Salmonella in a stool sample. The method may comprise: (a) an input means for receiving a request from a user to process the stool sample to detect Salmonella in the stool sample; (b) a mixing means for mixing the stool sample with a lysis buffer to obtain a mixture; (c) an incubating means for incubating the mixture at an incubation temperature greater than 15° C. for an incubation time period of no more than about 15 minutes; (d) an adding means for adding the mixture from (c) to a reaction vessel comprising reagents necessary for conducting nucleic acid amplification, the reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof, to obtain a reaction mixture; and (e) a reacting means for subjecting the reaction mixture in the reaction vessel to a plurality of series of primer extension reactions to generate amplified product(s) that is indicative of a presence of the target nucleic acid(s) in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In another aspect, the present disclosure provides a system for detecting Salmonella in a stool sample. The method may comprise an input unit that receives a request from a user to process the stool sample to detect Salmonella in the stool sample; and one or more computer processors operatively coupled to the input unit, wherein the one or more computer processors are individually or collectively programmed to: (a) mix the stool sample with a lysis buffer to obtain a mixture; (b) incubate the mixture at an incubation temperature greater than 15° C. for an incubation time period of no more than about 15 minutes; (c) add the mixture from (b) to a reaction vessel comprising reagents necessary for conducting nucleic acid amplification, the reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof, to obtain a reaction mixture; and (d) subject the reaction mixture in the reaction vessel to a plurality of series of primer extension reactions to generate amplified product(s) that is indicative of a presence of the target nucleic acid(s) in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In another aspect, the present disclosure provides a reaction mixture. The reaction mixture may comprise Salmonella, Salmonella lysate, or Salmonella nucleic acids; one or more primer sets, each capable of specifically binding to a target nucleic acid sequence from a Salmonella genome or a Salmonella transcriptome, or a variant thereof, for amplifying the target nucleic acid sequence in an amplification reaction to obtain amplification product(s); a deoxyribonucleic acid (DNA) polymerase and optionally a reverse transcriptase; nucleotides and analogs thereof capable of being incorporated by the DNA polymerase in an amplification reaction, and optionally, reporter agent(s) that yields a detectable signal indicative of a presence of the amplified product(s).

In some embodiments, the Salmonella nucleic acids are selected from a group consisting of genome DNA, cDNA, non-coding DNA, mRNA, rRNA, tRNA, siRNA, shRNA, miRNA, and combination thereof.

In some embodiments, the nucleotides and analogs thereof capable of being incorporated by the DNA polymerase in an amplification reaction are dNTPs.

In some embodiments, the one or more primer set includes a primer set comprising a forward primer as depicted in SEQ ID NO: 1 (TGCTCGTTTACGACCTGAATTA) and a reverse primer depicted in SEQ ID NO: 2 (ACACCAATATCGCCAGTACG).

In some embodiments, the reporter agent includes a sequence-specific oligonucleotide probe comprising a nucleic acid sequence as depicted in SEQ ID NO: 3 (TCTGGTTGATTTCCTGATCGCACTGA).

In some embodiments, the one or more primer set includes a primer set comprising a forward primer as depicted in SEQ ID NO: 4 (TCGTTTACGACCTGAATTAC) and a reverse primer depicted in SEQ ID NO: 5 (TAGAACGACCCCATAAACA).

In some embodiments, the reporter agent includes a sequence-specific oligonucleotide probe comprising a nucleic acid sequence as depicted in SEQ ID NO: 6 (CTGGTTGATTTCCTGATCGCACT).

In some embodiments, the one or more primer set includes a primer set comprising a forward primer as depicted in SEQ ID NO: 7 (CTCACCAGGAGATTACAACATGG) and a reverse primer depicted in SEQ ID NO: 8 (AGCTCAGACCAAAAGTGACCATC).

In some embodiments, the reporter agent includes a sequence-specific oligonucleotide probe comprising a nucleic acid sequence as depicted in SEQ ID NO: 9 (CACCGACGGCGAGACCGACTTT).

In another aspect, the present disclosure provides a kit for detecting Salmonella in a stool sample. The kit may comprise: one or more primer sets, each capable of specifically binding to a target nucleic acid sequence from a Salmonella genome or a Salmonella transcriptome, or a variant thereof, for amplifying the target nucleic acid sequence in an amplification reaction to obtain amplification product(s); a deoxyribonucleic acid (DNA) polymerase and optionally a reverse transcriptase; buffer(s) for a nucleic acid amplification; nucleotides and analogs thereof capable of being incorporated by the DNA polymerase in an amplification reaction; optionally, reporter agent(s) that yields a detectable signal indicative of a presence of the amplified product(s), and optionally instructions for using the one or more primers sets, DNA polymerase, and dNTPs to perform nucleic acid amplification to detect Salmonella in the stool sample.

The kit may further comprise a unique identifier which is extractable for information on one or more relevant parameters for conducting a primer extension reaction.

In some embodiments, the parameters are selected from the group consisting of the number of series of the primer extension reaction, the number of cycles in each series, denaturing condition, elongation condition, one or more primer sets, reporter agent(s), oligonucleotide probe(s), and combinations thereof.

In some embodiments, the unique identifier is a barcode.

In some embodiments, the unique identifier is an RFID tag.

In another aspect, the present disclosure provides a system for detecting Salmonella in a stool sample, the system may comprise: (a) a recognition module for recognizing information on one or more relevant parameters for conducting a primer extension reaction included in a kit for use in conjunction with the system; (b) an amplification module which, upon recognizing the information, automatically subjects a reaction mixture in a reaction vessel to a plurality of series of primer extension reactions to generate amplified product(s) that is indicative of a presence of target nucleic acid(s) in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition, wherein the reaction mixture is obtained by adding a lysate derived from the stool sample to a reaction vessel comprising reagents necessary for conducting nucleic acid amplification, the reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof.

The system may further comprise an output module for outputting information indicative of an amount and/or presence of the amplified product(s) to a recipient.

In some embodiments, the recognition module comprises a barcode scanning module for scanning a barcode on the kit for extraction of information.

In some embodiments, the recognition module comprises a RFID recognition module for recognizing a RFID tag on the kit for extraction of information.

In some embodiments, the parameters are selected from the group consisting of the number of series of the primer extension reaction, the number of cycles in each series, denaturing condition, elongation condition, one or more primer sets, reporter agent(s), oligonucleotide probe(s).

In some embodiments, upon recognizing the information, the recognition module communicates with the amplification, thereby transmitting the one or more relevant parameters to the amplification module for conducting the plurality of series of primer extension reactions.

The system may further comprise a detection module for detecting the amount and/or presence of the amplified product(s).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “Fig.” herein), of which:

FIG. 1 is schematic depicting an example system.

FIGS. 2A and 2B are graphs depicting results of example nucleic acid amplification reactions described in Example 1.

FIGS. 3A and 3B are graphs depicting results of example nucleic acid amplification reactions described in Example 1.

FIGS. 4A and 4B are graphs depicting results of example nucleic acid amplification reactions described in Example 2.

FIG. 5 is a graph depicting results of example nucleic acid amplification reactions described in Example 3.

FIGS. 6A and 6B are graphs depicting results of example nucleic acid amplification reactions described in Example 4.

FIGS. 7A and 7B are graphs depicting results of example nucleic acid amplification reactions described in Example 4.

FIGS. 8A and 8B are graphs depicting results of example nucleic acid amplification reactions described in Example 4.

FIGS. 9A and 9B are graphs depicting results of example nucleic acid amplification reactions described in Example 4.

FIGS. 10A and 10B are graphs depicting results of example nucleic acid amplification reactions described in Example 4.

FIG. 11 is a graph depicting results of example nucleic acid amplification reactions described in Example 5.

FIG. 12 is a graph depicting results of example nucleic acid amplification reactions described in Example 5.

FIG. 13 is a graph depicting results of example nucleic acid amplification reactions described in Example 7.

FIG. 14 is a graph depicting results of example nucleic acid amplification reactions described in Example 9.

FIGS. 15A and 15B are graphs depicting results of example nucleic acid amplification reactions described in Example 10.

FIGS. 16A and 16B are graphs depicting results of example nucleic acid amplification reactions described in Example 10.

FIG. 17 is a graph depicting results of nucleic acid amplification reactions described in Example 11.

FIG. 18 is a graph depicting results of nucleic acid amplification reactions described in Example 12.

FIG. 19A and FIG. 19B are graphs depicting results of nucleic acid amplification reactions described in Example 13.

FIG. 20 is a graph depicting results of nucleic acid amplification reactions described in Example 14.

FIG. 21 is a graph depicting results of nucleic acid amplification reactions described in Example 15.

FIG. 22A and FIG. 22B are graphs depicting results of nucleic acid amplification reactions described in Example 17.

FIG. 23A, FIG. 23B and FIG. 23C are graphs depicting results of nucleic acid amplification reactions described in Example 18.

FIG. 24A and FIG. 24B are graphs depicting results of nucleic acid amplification reactions described in Example 19.

FIG. 25A and FIG. 25B are graphs depicting results of nucleic acid amplification reactions described in Example 19.

FIG. 26A and FIG. 26B are graphs depicting results of nucleic acid amplification reactions described in Example 20.

FIG. 27 is a graph depicting results of nucleic acid amplification reactions described in Example 21.

FIG. 28A is a schematic of an example electronic display having an example user interface.

FIG. 28B is a schematic of an example electronic display having an example user interface.

FIG. 29 is a graph depicting results of various nucleic acid amplification reactions.

FIG. 30A and FIG. 30B are graphs depicting results of various nucleic acid amplification reactions.

FIG. 31A and FIG. 31B are graphs depicting results of various nucleic acid amplification reactions.

FIG. 32A and FIG. 32B are graphs depicting results of various nucleic acid amplification reactions.

FIG. 33 is a graph depicting results of various nucleic acid amplification reactions.

FIG. 34 is a graph depicting results of various nucleic acid amplification reactions.

FIG. 35 (panel A) and FIG. 35 (panel B) are graphs depicting results of various nucleic acid amplification reactions.

FIG. 36 (panel A) and FIG. 36 (panel B) are graphs depicting results of various nucleic acid amplification reactions.

FIG. 37 (panel A) and FIG. 37 (panel B) are graphs depicting results of various nucleic acid amplification reactions.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an” and “the” include plural references unless the context clearly dictates otherwise. For example, the term “a cell” includes a plurality of cells, including mixtures thereof.

As used in the specification and claims, the term “about” refers to a range that is no more than 10% greater than or less than a stated numerical value unless the context dictates otherwise. For example, the range may be 10%, 9%, 8%, 7%, 6%, 5%, 4%, 3%, 2%, 1%, or 0.5% greater than or less than a stated numerical value.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase.

As used herein, the term “cycle threshold” or “Ct” generally refers to the cycle during thermocycling in which an increase in a detectable signal due to amplified product reaches a statistically significant level above background signal.

As used herein, the terms “incubating” and “incubation” are used interchangeably and generally refer to keeping a sample, a mixture or a solution at certain temperature for a certain period of time, with or without shaking or stirring. An “incubation temperature” generally refers to a temperature at which incubation is permitted to occur. An “incubation time period” generally refers to an amount of time allotted for incubation to occur.

As used herein, the terms “denaturing” and “denaturation” are used interchangeably and generally refer to the full or partial unwinding of the helical structure of a double-stranded nucleic acid, and in some cases the unwinding of the secondary structure of a single stranded nucleic acid. Denaturation may include the inactivation of the cell wall(s) of a pathogen or the shell of a virus, and the inactivation of the protein(s) of inhibitors. Conditions at which denaturation may occur include a “denaturation temperature” that generally refers to a temperature at which denaturation is permitted to occur and a “denaturation duration” that generally refers to an amount of time allotted for denaturation to occur.

As used herein, the term “elongation” generally refers to the incorporation of nucleotides to a nucleic acid in a template directed fashion. Elongation may occur via the aid of an enzyme, such as, for example, a polymerase or reverse transcriptase. Conditions at which elongation may occur include an “elongation temperature” that generally refers to a temperature at which elongation is permitted to occur and an “elongation duration” that generally refers to an amount of time allotted for elongation to occur.

As used herein, the term “nucleic acid” generally refers to a polymeric form of nucleotides of any length, either deoxyribonucleotides or ribonucleotides, or analogs thereof. Nucleic acids may have any three dimensional structure, and may perform any function, known or unknown. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non nucleotide components. A nucleic acid may be further modified after polymerization, such as by conjugation or binding with a reporter agent.

As used herein, the term “primer extension reaction” generally refers to the denaturing of a double-stranded nucleic acid, binding of a primer to one or both strands of the denatured nucleic acid, followed by elongation of the primer(s). In some cases, a template nucleic acid may be single-stranded (e.g., partially single-stranded) without denaturation, and a primer may bind to the single-stranded nucleic acid, followed by elongation of the primer(s).

As used herein, the term “reaction mixture” generally refers to a composition comprising reagents necessary to complete nucleic acid amplification (e.g., DNA amplification, RNA amplification), with non-limiting examples of such reagents that include primer sets having specificity for target RNA or target DNA, DNA produced from reverse transcription of RNA, a DNA polymerase, a reverse transcriptase (e.g., for reverse transcription of RNA), suitable buffers (including zwitterionic buffers), co-factors (e.g., divalent and monovalent cations), dNTPs, and other enzymes (e.g., uracil-DNA glycosylase (UNG)), etc). In some cases, reaction mixtures can also comprise one or more reporter agents.

As used herein, a “reporter agent” generally refers to a composition that yields a detectable signal, the presence or absence of which can be used to detect the presence of amplified product.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogues thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the term “subject,” generally refers to an entity or a medium that has testable or detectable genetic information. A subject can be a person or individual. A subject can be a vertebrate, such as, for example, a mammal. Non-limiting examples of mammals include murines, simians, humans, farm animals, sport animals, and pets. Other examples of subjects include food, plant, soil, and water.

Salmonella is the generic name for a genus of Gram-negative bacteria belonging to the family Enterobacteriaceae. The genus Salmonella comprises two Salmonella bacteria, that is, Salmonella enterica and Salmonella bongori. Salmonella enterica can be classified into 6 subspecies and more than 2,500 serotypes. As used herein, the term “Salmonella” generally refers to all bacteria belonging to the genus Salmonella. The Salmonella genus include numerous species including, but not limited to Salmonella typhi, Salmonella typhimurium, Salmonella enteritidis, Salmonella choleraesuis, Salmonella paratyphi, Salmonella arizonae, and the like. Unless specifically stated otherwise, all these Salmonella are encompassed in the term “Salmonella” as recited herein.

In one aspect, the present disclosure provides a method for detecting a target nucleic acid molecule in a biological sample. The method may comprise: (a) mixing said biological sample with a lysis buffer to obtain a mixture; (b) incubating said mixture at a temperature from about 15° C. to 70° C. for a period of time of no more than about 15 minutes; (c) adding said mixture from (b) to a reaction vessel comprising reagents necessary for conducting nucleic acid amplification, said reagents comprising (i) a deoxyribonucleic acid (DNA) polymerase, and (ii) a primer set for said target nucleic acid molecule, to obtain a reaction mixture; and (d) subjecting said reaction mixture in said reaction vessel to multiple cycles of a primer extension reaction to generate amplified product(s) that is indicative of a presence of said target nucleic acid molecule, each cycle comprising (i) incubating said reaction mixture at a denaturing temperature for a denaturing duration that is less than or equal to 60 seconds, followed by (ii) incubating said reaction mixture at an elongation temperature for an elongation duration that is less than or equal to 60 seconds, thereby amplifying said target nucleic acid molecule. The amplified product may be DNA product.

In another aspect, the present disclosure provides a method for detecting a target nucleic acid molecule in a biological sample. The method may comprise: (a) mixing said biological sample with a lysis buffer to obtain a mixture; (b) incubating said mixture at a temperature from about 15° C. to 70° C. for a period of time of no more than about 15 minutes; (c) adding said mixture from (b) to a reaction vessel comprising reagents necessary for conducting nucleic acid amplification, said reagents comprising (i) a deoxyribonucleic acid (DNA) polymerase and optionally a reverse transcriptase, and (ii) a primer set for said target nucleic acid molecule, to obtain a reaction mixture; and (d) subjecting said reaction mixture in said reaction vessel to a plurality of series of primer extension reactions to generate amplified product(s) that is indicative of a presence of said target nucleic acid molecule in said sample, each series comprising two or more cycles of (i) incubating said reaction mixture under a denaturing condition characterized by a denaturing temperature and a denaturing duration, followed by (ii) incubating said reaction mixture under an elongation condition characterized by an elongation temperature and an elongation duration, wherein an individual series differs from at least one other individual series of said plurality with respect to said denaturing condition and/or said elongation condition. The amplified product may be DNA product.

In one aspect, the present disclosure provides a method for detecting a target nucleic acid molecule in a biological sample. The method may comprise (a) mixing said biological sample with a lysis buffer to obtain a mixture, wherein said biological sample includes a stool sample or milk sample; (b) incubating said mixture at an incubation temperature for an incubation time period; (c) adding said mixture from (b) to a reaction vessel comprising reagents necessary for conducting nucleic acid amplification, said reagents comprising (i) a deoxyribonucleic acid (DNA) polymerase, and (ii) a primer set for said target nucleic acid molecule, to obtain a reaction mixture; and (d) subjecting said reaction mixture in said reaction vessel to multiple cycles of a primer extension reaction to generate amplified product(s) that is indicative of a presence of said target nucleic acid molecule, each cycle comprising (i) incubating said reaction mixture at a denaturing temperature for a denaturing duration that is less than or equal to 60 seconds, followed by (ii) incubating said reaction mixture at an elongation temperature for an elongation duration that is less than or equal to 60 seconds, thereby amplifying said target nucleic acid molecule. The amplified product may be DNA product.

In another aspect, the present disclosure provides a method for detecting a target nucleic acid molecule in a biological sample. The method may comprise: (a) mixing said biological sample with a lysis buffer to obtain a mixture, wherein said biological sample includes a stool sample or milk sample; (b) incubating said mixture at an incubation temperature for an incubation time period; (c) adding said mixture from (b) to a reaction vessel comprising reagents necessary for conducting nucleic acid amplification, said reagents comprising (i) a deoxyribonucleic acid (DNA) polymerase and optionally a reverse transcriptase, and (ii) a primer set for said target nucleic acid molecule, to obtain a reaction mixture; and (d) subjecting said reaction mixture in said reaction vessel to a plurality of series of primer extension reactions to generate amplified product(s) that is indicative of a presence of said target nucleic acid molecule in said sample, each series comprising two or more cycles of (i) incubating said reaction mixture under a denaturing condition characterized by a denaturing temperature and a denaturing duration, followed by (ii) incubating said reaction mixture under an elongation condition characterized by an elongation temperature and an elongation duration, wherein an individual series differs from at least one other individual series of said plurality with respect to said denaturing condition and/or said elongation condition. The amplified product may be DNA product.

In another aspect, the present disclosure provides a method for detecting Salmonella in a biological sample. The method may comprise: (a) mixing said biological sample with a lysis buffer to obtain a mixture; (b) incubating said mixture at an incubation temperature greater than 15° C. for an incubation time period of no more than about 15 minutes; (c) adding said mixture from (b) to a reaction vessel comprising reagents necessary for conducting nucleic acid amplification, said reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof, to obtain a reaction mixture; and (d) subjecting said reaction mixture in said reaction vessel to a plurality of series of primer extension reactions to generate amplified product(s) that is indicative of a presence of said target nucleic acid(s) in said sample, each series comprising two or more cycles of (i) incubating said reaction mixture under a denaturing condition characterized by a denaturing temperature and a denaturing duration, followed by (ii) incubating said reaction mixture under an elongation condition characterized by an elongation temperature and an elongation duration, wherein an individual series differs from at least one other individual series of said plurality with respect to said denaturing condition and/or said elongation condition. The biological sample may be a stool sample. The biological sample may be a cell culture sample. The amplified product may be DNA product.

In any of the various aspects of the present disclosure, the “incubation temperature” may be from about 10° C. to 75° C., for example, at a temperature that is from about 10° C. to 70° C., from about 15° C. to 65° C., from about 15° C. to 60° C., from about 15° C. to 55° C., from about 20° C. to 50° C., from about 20° C. to 45° C., from about 20° C. to 40° C., from about 20° C. to 35° C., from about 20° C. to 30° C., from about 20° C. to 25° C., or from about 25° C. to 30° C.

Alternatively, the incubation temperature may be greater than 15° C., for example, at a temperature that is greater than about 20° C., greater than about 25° C., greater than about 30° C., greater than about 35° C., greater than about 40° C., greater than about 45° C., greater than about 50° C., greater than about 55° C., greater than about 60° C., greater than about 65° C., greater than about 70° C., greater than about 75° C., greater than about 80° C., greater than about 85° C., greater than about 90° C., greater than about 91° C., greater than about 92° C., greater than about 93° C., greater than about 94° C., greater than about 95° C., greater than about 96° C., greater than about 97° C., greater than about 98° C., greater than about 99° C., greater than about 100° C., or at a temperature that is from about 15° C. to 95° C., for example, at a temperature that is from about 20° C. to 90° C., from about 25° C. to 85° C., from about 30° C. to 80° C., from about 40° C. to 70° C., from about 40° C. to 95° C., from about 45° C. to 90° C., from about 50° C. to 85° C., from about 55° C. to 80° C., from about 60° C. to 75° C., from about 65° C. to 75° C., or from about 65° C. to 70° C.

In any of the various aspects of the present disclosure, the “incubation time period” may be no more than about 20 minutes. For example, the “incubation time period” may be no more than about 19 minutes, no more than about 18 minutes, no more than about 17 minutes, no more than about 16 minutes, no more than about 15 minutes, no more than about 14 minutes, no more than about 13 minutes, no more than about 12 minutes, no more than about 11 minutes, no more than about 10 minutes, no more than about 9 minutes, no more than about 8 minutes, no more than about 7 minutes, no more than about 6 minutes, no more than about 5 minutes, no more than about 4 minutes, no more than about 3 minutes, no more than about 2 minutes, no more than about 1 minute, no more than about 50 seconds, no more than about 40 seconds, no more than about 30 seconds, no more than about 20 seconds, no more than about 15 seconds, or no more than about 10 seconds.

In any of the various aspects of the present disclosure, prior to mixing the biological sample with a lysis buffer, the biological sample may be suspended in solution to obtain a homogenized preparation comprising the biological sample. The solution may be a suspension buffer. The suspension buffer may comprise NaCl, PBS and/or HEPES. Suspension buffer that can be used in accordance with the present disclosure includes, but is not limited to sodium chloride solutions of about 0.9%, phosphate buffers, lactated Ringer's solution, acetated Ringer's solution, phosphate buffered saline, citrate buffers, a sodium bicarbonate buffer, a borate buffer, a Tris buffer, a histidine buffer, a HEPES buffer, a MOPS buffer, glycine buffers, N-glycylglycine buffers, and combinations thereof.

In any of the various aspects of the present disclosure, the homogenized preparation may be incubated at a temperature that is greater than about 40° C., for example, at a temperature that is greater than about 45° C., greater than about 50° C., greater than about 55° C., greater than about 60° C., greater than about 65° C., greater than about 70° C., greater than about 75° C., greater than about 80° C., greater than about 85° C., greater than about 90° C., greater than about 91° C., greater than about 92° C., greater than about 93° C., greater than about 94° C., greater than about 95° C., greater than about 96° C., greater than about 97° C., greater than about 98° C., greater than about 99° C., or greater than about 100° C.

In any of the various aspects of the present disclosure, the homogenized preparation may be incubated for a time period of no more than about 20 minutes, for example, for a time period of no more than about 19 minutes, no more than about 18 minutes, no more than about 17 minutes, no more than about 16 minutes, no more than about 15 minutes, no more than about 14 minutes, no more than about 13 minutes, no more than about 12 minutes, no more than about 11 minutes, no more than about 10 minutes, no more than about 9 minutes, no more than about 8 minutes, no more than about 7 minutes, no more than about 6 minutes, no more than about 5 minutes, no more than about 4 minutes, no more than about 3 minutes, no more than about 2 minutes, no more than about 1 minute, no more than about 50 seconds, no more than about 40 seconds, no more than about 30 seconds, no more than about 20 seconds, no more than about 15 seconds, or no more than about 10 seconds.

In some embodiments, prior to being mixed with a lysis buffer, the biological sample is subjected to centrifugation to yield a solution comprising the biological sample and a pellet. In some embodiments, prior to being mixed with a lysis buffer, the biological sample is subjected to centrifugation to yield a pellet comprising the biological sample and a supernatant.

In some embodiments, after incubating the mixture of the biological sample and the lysis buffer, the mixture may be subjected to centrifugation to yield a supernatant comprising the biological sample. Then, the supernatant may serve as the mixture in the subsequent steps. For example, the supernatant may be added to a reaction vessel comprising reagents necessary for conducting nucleic acid amplification.

In some cases, before conducting the primer extension reactions, the target nucleic acid molecules may be subjected to one or more denaturing conditions. The one or more denaturing conditions may be selected from a denaturing temperature profile and a denaturing agent.

In some cases, before conducting the primer extension reactions, the biological sample may be pre-heated at a pre-heating temperature from 90° C. to 100° C. for a pre-heating duration of no more than 10 minutes. In some embodiments, the pre-heating duration is no more than 1 minute.

In any of the various aspects of the present disclosure, the mixture of the biological sample with the lysis buffer may be added to the reaction vessel comprising reagents necessary for conducting nucleic acid amplification without undergoing DNA or ribonucleic acid (RNA) extraction. In some cases, the mixture may be added to the reaction vessel without undergoing purification. In certain cases, the mixture may be added to the reaction vessel without undergoing DNA or RNA concentration.

In any of the various aspects of the present disclosure, the mixture of the biological sample with the lysis buffer may be incubated at a temperature that is from about 10° C. to 75° C., for example, at a temperature that is from about 10° C. to 70° C., from about 15° C. to 65° C., from about 15° C. to 60° C., from about 15° C. to 55° C., from about 20° C. to 50° C., from about 20° C. to 45° C., from about 20° C. to 40° C., from about 20° C. to 35° C., from about 20° C. to 30° C., from about 20° C. to 25° C., or from about 25° C. to 30° C.

In any of the various aspects of the present disclosure, the mixture of the biological sample with the lysis buffer may be incubated for a period of time that is no more than about 20 minutes. For example, the period of time in (b) may be no more than about 19 minutes, no more than about 18 minutes, no more than about 17 minutes, no more than about 16 minutes, no more than about 15 minutes, no more than about 14 minutes, no more than about 13 minutes, no more than about 12 minutes, no more than about 11 minutes, no more than about 10 minutes, no more than about 9 minutes, no more than about 8 minutes, no more than about 7 minutes, no more than about 6 minutes, no more than about 5 minutes, no more than about 4 minutes, no more than about 3 minutes, no more than about 2 minutes, no more than about 1 minute, no more than about 50 seconds, no more than about 40 seconds, no more than about 30 seconds, no more than about 20 seconds, no more than about 15 seconds, or no more than about 10 seconds.

In any of the various aspects of the present disclosure, nucleic acid from a biological sample obtained from a subject is amplified. The biological sample may be obtained directly from a source thereof. For example, the biological sample may be obtained directly from a source thereof without pre-culturing, non-selective enrichment, selective enrichment, plating on differential medium, and/or presumptive biomedical identification. “Pre-culturing” generally refers to a process for expanding one or more target species (e.g., microorganisms) in a sample, or for increasing the number thereof, prior to performing methods of the present disclosure. “Non-selective enrichment” generally refers to a process of increasing the amount of a majority or all of the species (e.g., microorganisms) in a mixed population non-selectively. “Selective enrichment” generally refers to a process of increasing the proportion and/or amount of one or more specific species (e.g., microorganisms) in a mixed population while inhibiting other species. Such inhibition may result due to medium constituents such as compounds which are selectively toxic, as well as the end-products of microbial metabolism produced by organisms which utilize the medium constituents. “Differential medium” generally refers to a medium that includes one or more added indicator(s) that allows for the differentiation of particular chemical reactions occurring during growth. “Presumptive biomedical identification” generally refers to preliminary identification of a microorganism based on observation such as colony characteristics, growth on primary isolation media, gram stain results, etc.

In some embodiments, the biological sample is cultivated for microbial proliferation. In some embodiments, prior to being mixed with a lysis buffer, the biological sample is subjected to enrichment culturing conditions for a culturing time period. The enrichment culturing conditions may comprise culturing the biological sample in a suitable culture medium (e.g., tryptic soy broth, modified tryptic soy broth, tryptone, nutrient broth, L-broth, gram negative broth, peptone, tryptic soy broth with yeast, or Salmonella medium) at a suitable temperature (e.g., from 23° C. to 40° C., such as 25° C., 30° C., 35° C., or 37° C.) with or without shaking. In some embodiment, the culture medium is a Salmonella medium which favors proliferation of Salmonella as compared to other bacteria. Exemplary Salmonella medium includes Bismuth Sulfite agar (BS), xylose lysine deoxycholate agar (XLD), selenite brilliant green sulfa medium (SBG), but is not limited thereto. The culturing time period may be from about 0.5 hour to 10 hours, e.g., about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, or about 10 hours. In some embodiments, the culturing time period is no more than about 7 hours, e.g., no more than about 6.5 hours, no more than about 6 hours, no more than about 5.5 hours, no more than about 5 hour, no more than about 4.5 hour, no more than about 4 hours, no more than about 3.5 hours, no more than about 3 hours, no more than about 2.5 hours, no more than about 2 hours, no more than about 1.5 hours, no more than about 1 hour, or no more than about 0.5 hour.

In some embodiments, prior to and/or after being subjected to enrichment culturing conditions for a culturing time period, the biological sample is subjected to centrifugation to yield a solution comprising the biological sample and a pellet. In some embodiments, prior to and/or after being subjected to enrichment culturing conditions for a culturing time period, the biological sample is subjected to centrifugation to yield a pellet comprising the biological sample and a supernatant.

In some embodiments, after being subjected to enrichment culturing conditions for a culturing time period, the biological sample is mixed with the lysis buffer without selective enrichment, plating on differential medium, and/or presumptive biomedical identification.

In some embodiments, after the biological sample has been subjected to enrichment culturing conditions for a culturing time period, a lysis buffer may be added to the mixture. The lysis buffer may be alkaline. For example, the lysis buffer comprises NaOH. The lysis buffer may have a pH from about 7 to 14, such as from about 8 to 13, from about 9 to 12, from about 10 to 11. For example, the lysis buffer may have a pH of about 7, 7.5, 8, 8.5, 9, 9.5, 10, 10.5, 11, 11.5, 12, 12.5, 13, 13.5, or 14.

In any of the various aspects, the present disclosure involves obtaining a biological sample. In some cases, the biological sample is obtained directly from the subject. A biological sample obtained directly from a subject generally refers to a biological sample that has not been further processed after being obtained from the subject, with the exception of any approach used to collect the biological sample from the subject for further processing. For example, blood is obtained directly from a subject by accessing the subject's circulatory system, removing the blood from the subject (e.g., via a needle), and entering the removed blood into a receptacle. The receptacle may comprise reagents (e.g., anti-coagulants) such that the blood sample is useful for further analysis. In another example, a swab may be used to access epithelial cells on an oropharyngeal surface of the subject. In a further example, a swab may be used to access stool samples of the subject. After obtaining the biological sample from the subject, the swab containing the biological sample can be contacted with a fluid (e.g., a buffer) to collect the biological fluid from the swab.

In some embodiments, the biological sample is a stool sample. In some embodiments, the stool sample is a solid stool sample. In some embodiments, the stool sample is a liquid stool sample. In some embodiments, the solid stool sample may be suspended in a suitable buffer as a suspended stool sample. In some embodiments, the liquid stool sample may be a watery diarrhea sample.

In some embodiments, the stool sample is obtained by a swab. For example, the swab may be used to scratch the surface of the solid stool sample to obtain the stool sample. Alternatively, the swab may be dipped into a liquid stool sample or a suspended stool sample to obtain the stool sample. The swab may be a sterile swab. The swab may be sterile flocked swab.

There may be other ways to obtain the stool sample. For example, a liquid stool sample or a suspended stool sample may be obtained by a pipette, a micropipette, an injector, or the like. For example, a solid stool sample may be obtained by using a medicine spoon, a pipette tip, tweezers, or the like.

In any of the various aspects of the present disclosure, the weight of the biological sample may be about 50 mg to about 5 g. For example, the weight of the biological sample may be about 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1.0 g, 1.1 g, 1.2 g, 1.3 g, 1.4 g, 1.5 g, 1.6 g, 1.7 g, 1.8 g, 1.9 g, 2.0 g, 2.5 g, 3.0 g, 3.5 g, 4.0 g, 4.5 g, or 5.0 g, or may be any value or range between any two of the aforesaid numeric values.

In any of the various aspects of the present disclosure, the biological sample may be a liquid or a suspension. The volume of the liquid biological sample or the suspended biological sample may be about 50 μl to about 5 ml, for example, the volume of the biological sample may be about 50 μl, 100 μl, 150 μl, 200 μl, 250 μl, 300 μl, 350 μl, 400 μl, 450 μl, 500 μl, 550 μl, 600 μl, 650 μl, 700 μl, 750 μl, 800 μl, 850 μl, 900 μl, 950 μl, 1.0 ml, 1.1 ml, 1.2 ml, 1.3 ml, 1.4 ml, 1.5 ml, 1.6 ml, 1.7 ml, 1.8 ml, 1.9 ml, 2.0 ml, 2.5 ml, 3.0 ml, 3.5 ml, 4.0 ml, 4.5 ml, or 5.0 ml, or may be any value or range between any two of the aforesaid numeric values.

In another example, food (e.g., milk) may be obtained directly from a source thereof (e.g., a container comprising the food) by accessing the food in the source, removing the food from the container (e.g., by pipetting), and entering the removed food into a receptacle.

In some embodiments, a biological sample has not been purified when provided in a reaction vessel. In some embodiments, the nucleic acid of a biological sample has not been extracted when the biological sample is provided to a reaction vessel. For example, the RNA or DNA in a biological sample may not be extracted from the biological sample when providing the biological sample to a reaction vessel. Moreover, in some embodiments, a target nucleic acid (e.g., a target RNA or target DNA) present in a biological sample may not be concentrated prior to providing the biological sample to a reaction vessel.

In any of the various aspects of the present disclosure, the mixture of the biological sample and the lysis buffer may be added to the reaction vessel without being subject to DNA or RNA extraction. In some cases, the mixture may be added to the reaction vessel without being purified. In some cases, the mixture may be added to the reaction vessel without being subject to DNA or RNA concentration. The reaction vessel may be one that comprises reagents necessary for conducting nucleic acid amplification.

For example, after incubation of the mixture of the biological sample and the lysis buffer as described elsewhere herein, the mixture is added to the reaction vessel without being purified. For example, the mixture is added to the reaction vessel without being subject to DNA or RNA extraction. For example, the mixture is added to the reaction vessel without being subject to DNA or RNA concentration. The reaction vessel may be one that comprises reagents necessary for conducting nucleic acid amplification.

For example, after centrifuging the mixture of the biological sample and the lysis buffer to obtain a supernatant as described elsewhere herein, the supernatant is added to the reaction vessel without being purified. For example, the supernatant is added to the reaction vessel without being subject to DNA or RNA extraction. For example, the supernatant is added to the reaction vessel without being subject to DNA or RNA concentration. The reaction vessel may be one that comprises reagents necessary for conducting nucleic acid amplification.

Any suitable biological sample that comprises nucleic acid may be obtained from a subject. A biological sample may be solid matter (e.g., biological tissue) or may be a fluid (e.g., a biological fluid). In general, a biological fluid can include any fluid associated with living organisms. Non-limiting examples of a biological sample include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, microbiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, lymphatic fluids, and/or other excretions or body tissues. In an example, the biological sample is a stool sample.

In other cases, the biological sample may be from a soil or food sample. For example, the food sample may be a dairy sample and in some cases, the diary sample may include milk.

A biological sample may be obtained from a subject using various approaches. Non-limiting examples of approaches to obtain a biological sample directly from a subject include accessing the circulatory system (e.g., intravenously or intra-arterially via a syringe or other needle), collecting a secreted biological sample (e.g., feces, urine, sputum, saliva, etc.), surgically (e.g., biopsy), swabbing (e.g., buccal swab, oropharyngeal swab, rectal swab), pipetting, and breathing. Moreover, a biological sample may be obtained from any anatomical part of a subject where a desired biological sample is located. In some embodiments, the biological sample may be obtained from a container thereof, e.g., a container (e.g., a bag, a box or a bottle) comprising food (e.g., milk) or soil. Soil may be mixture of minerals, organic matter, gases, liquids, and in some cases organisms.

In any of the various aspects, a target nucleic acid is amplified to generate an amplified product. A target nucleic acid may be a target RNA or a target DNA. The target nucleic acid molecule may be associated with a disease. In cases where the target nucleic acid is a target RNA, the target RNA may be any type of RNA, including types of RNA described elsewhere herein. In some embodiments, the target RNA is viral RNA. In some embodiments, the viral RNA may be pathogenic to the subject. Non-limiting examples of pathogenic viral RNA include human immunodeficiency virus I (HIV I), human immunodeficiency virus II (HIV II), orthomyxoviruses, Ebola virus, Dengue virus, influenza viruses (e.g., H1N1, H3N2, H7N9, or H5N1), hepesvirus, hepatitis A virus, hepatitis B virus, hepatitis C (e.g., armored RNA-HCV virus) virus, hepatitis D virus, hepatitis E virus, hepatitis G virus, Epstein-Barr virus, mononucleosis virus, cytomegalovirus, SARS virus, West Nile Fever virus, polio virus, measles, enterovirus (such as Coxsackie virus, e.g., Coxsackie virus A16), and norovirus (e.g., norovirus GI or norovirus GII).

In some embodiments, the target RNA is a Salmonella mRNA. In some embodiments, the target RNA may be selected from mRNA with high expression abundance in Salmonella. In some embodiments, the target RNA is selected from the group consisting of ttr mRNA, invA mRNA, prgK mRNA, RpoS mRNA, RpoD mRNA, or combinations thereof. In some embodiments, the target RNA is invA mRNA.

In cases where the target nucleic acid is a target DNA, the target DNA may be any type of DNA, including types of DNA described elsewhere herein. In some embodiments, the target DNA is viral DNA. In some embodiments, the viral DNA may be pathogenic to the subject. Non-limiting examples of DNA viruses include herpes simplex virus, smallpox, adenovirus (e.g., Adenovirus Type 55, Adenovirus Type 7) and Varicella virus (e.g., chickenpox).

In some cases, a target DNA may be a bacterial DNA. The bacterial DNA may be from a bacterium pathogenic to the subject. The pathogenic bacterium may be a gram-positive or gram-negative pathogenic bacterium. For example, the pathogenic bacterium may be selected from the group consisting of Staphylococcus aureus, Listeria monocytogenes, Escherichia coli, Enterobacter sakazakii, Vibrio Parahemolyticus, Shigella spp., and Mycobacterium tuberculosis (a bacterium known to cause tuberculosis). In some embodiments, the pathogenic bacterium is Salmonella. In some cases, a target DNA may be a DNA from a pathogenic protozoan, such as, for example one or more protozoans of the Plasmodium type that can cause Malaria.

In some embodiments, the target DNA is a Salmonella DNA. In some embodiments, the target DNA may be selected from genes specific to and/or conservative among Salmonella. In some embodiments, the target DNA is selected from the group consisting of ttr gene, invA gene, prgK gene, RpoS gene, RpoD gene, or combinations thereof. In some embodiments, the target DNA is ttr gene.

In any of the various aspects of the present disclosure, where the biological sample is suspended in the suspension buffer to yield a homogenized preparation, the biological sample may be at a ratio to the suspension buffer of about 5:1 (wt/vol) to about 1:500 (wt/vol), such as about 1:1 (wt/vol) to about 1:100 (wt/vol). For example, the ratio of the biological sample to the suspension buffer may be about 5:1 (wt/vol), about 4:1 (wt/vol), about 3:1 (wt/vol), about 2:1 (wt/vol), about 1:1 (wt/vol), about 1:2 (wt/vol), about 1:3 (wt/vol), about 1:4 (wt/vol), about 1:5 (wt/vol), about 1:6 (wt/vol), about 1:7 (wt/vol), about 1:8 (wt/vol), about 1:9 (wt/vol), about 1:10 (wt/vol), about 1:20 (wt/vol), about 1:30 (wt/vol), about 1:40 (wt/vol), about 1:50 (wt/vol), about 1:60 (wt/vol), about 1:70 (wt/vol), about 1:80 (wt/vol), about 1:90 (wt/vol), about 1:100 (wt/vol), about 1:110 (wt/vol), about 1:120 (wt/vol), about 1:130 (wt/vol), about 1:140 (wt/vol), about 1:150 (wt/vol), about 1:160 (wt/vol), about 1:170 (wt/vol), about 1:180 (wt/vol), about 1:190 (wt/vol), about 1:200 (wt/vol), about 1:250 (wt/vol), about 1:300 (wt/vol), about 1:350 (wt/vol), about 1:400 (wt/vol), about 1:450 (wt/vol), or about 1:500 (wt/vol).

In any of the various aspects of the present disclosure, where the lysis buffer is added to the homogenized preparation, the lysis buffer may be at a ratio to the homogenized preparation of about 50:1 (vol/vol) to about 1:50 (vol/vol), such as about 5:1 (vol/vol) to about 1:5 (vol/vol). For example the ratio of the lysis to the homogenized preparation may be about 50:1 (vol/vol), 40:1 (vol/vol), 30:1 (vol/vol), 20:1 (vol/vol), 10:1 (vol/vol), 9:1 (vol/vol), 8:1 (vol/vol), 7:1 (vol/vol), 6:1 (vol/vol), 5:1 (vol/vol), about 4:1 (vol/vol), about 3:1 (vol/vol), about 2:1 (vol/vol), about 1:1 (vol/vol), about 1:2 (vol/vol), about 1:3 (vol/vol), about 1:4 (vol/vol), about 1:5 (vol/vol), about 1:6 (vol/vol), about 1:7 (vol/vol), about 1:8 (vol/vol), about 1:9 (vol/vol), about 1:10 (vol/vol), about 1:20 (vol/vol), about 1:30 (vol/vol), about 1:40 (vol/vol), or about 1:50 (vol/vol).

In any of the various aspects of the present disclosure, a biological sample obtained from a subject or the mixture, supernatant, or homogenized preparation derived from the biological sample as described elsewhere herein is provided with reagents necessary for nucleic acid amplification in a reaction vessel to obtain a reaction mixture. Alternatively or additionally, any mixture, supernatant, or suspension obtained from a biological sample may be provided with reagents necessary for nucleic acid amplification in a reaction vessel to obtain a reaction mixture. Any suitable reaction vessel may be used. In some embodiments, a reaction vessel comprises a body that can include an interior surface, an exterior surface, an open end, and an opposing closed end. In some embodiments, a reaction vessel may comprise a cap. The cap may be configured to contact the body at its open end, such that when contact is made the open end of the reaction vessel is closed. In some cases, the cap is permanently associated with the reaction vessel such that it remains attached to the reaction vessel in open and closed configurations. In some cases, the cap is removable, such that when the reaction vessel is open, the cap is separated from the reaction vessel. In some embodiments, a reaction vessel may be sealed, in some cases hermetically sealed.

A reaction vessel may be of varied size, shape, weight, and configuration. In some examples, a reaction vessel may be round or oval tubular shaped. In some embodiments, a reaction vessel may be rectangular, square, diamond, circular, elliptical, or triangular shaped. A reaction vessel may be regularly shaped or irregularly shaped. In some embodiments, the closed end of a reaction vessel may have a tapered, rounded, or flat surface. Non-limiting examples of types of a reaction vessel include a tube, a well, a capillary tube, a cartridge, a cuvette, a centrifuge tube, or a pipette tip. Reaction vessels may be constructed of any suitable material with non-limiting examples of such materials that include glasses, metals, plastics, and combinations thereof.

In some embodiments, a reaction vessel is part of an array of reaction vessels. An array of reaction vessels may be particularly useful for automating methods and/or simultaneously processing multiple samples. For example, a reaction vessel may be a well of a microwell plate comprised of a number of wells. In another example, a reaction vessel may be held in a well of a thermal block of a thermocycler, wherein the block of the thermal cycle comprises multiple wells each capable of receiving a sample vessel. An array comprised of reaction vessels may comprise any appropriate number of reaction vessels. For example, an array may comprise at least 2, 4, 6, 8, 10, 12, 14, 16, 18, 20, 25, 35, 48, 96, 144, 384, or more reaction vessels. A reaction vessel part of an array of reaction vessels may also be individually addressable by a fluid handling device, such that the fluid handling device can correctly identify a reaction vessel and dispense appropriate fluid materials into the reaction vessel. Fluid handling devices may be useful in automating the addition of fluid materials to reaction vessels.

In some embodiments, a reaction vessel may comprise multiple thermal zones. Thermal zones within a reaction vessel may be achieved by exposing different regions of the reaction vessel to different temperature cycling conditions. For example, a reaction vessel may comprise an upper thermal zone and a lower thermal zone. The upper thermal zone may be capable of a receiving a biological sample and reagents necessary to obtain a reaction mixture for nucleic acid amplification. The reaction mixture can then be subjected to a first thermocycling protocol. After a desired number of cycles, for example, the reaction mixture can slowly, but continuously leak from the upper thermal zone to the lower thermal zone. In the lower thermal zone, the reaction mixture is then subjected to a desired number of cycles of a second thermocycling protocol different from that in the upper thermal zone. Such a strategy may be particularly useful when nested PCR is used to amplify DNA. In some embodiments, thermal zones may be created within a reaction vessel with the aid of thermal sensitive layering materials within the reaction vessels. In such cases, heating of the thermal sensitive layering materials may be used to release reaction mixtures from one thermal zone to the next. In some embodiments, the reaction vessel comprises 2, 3, 4, 5, 6, 7, 8, 9, 10, 11, 12, 13, 14, 15 or more thermal zones.

In some embodiments, a reaction vessel comprising thermal zones may be used for processing of a biological sample prior to nucleic acid amplification. For example, a lysis agent may be added to a first thermal zone of a reaction vessel prior to adding a biological sample and reagents necessary for nucleic acid amplification. When the biological sample and reagents are added to the reaction vessel comprising the lysis agent, a reaction mixture capable of lysing species (e.g., cells or viral particles) within the biological is obtained. Alternatively, a lysis agent can be added to the first thermal zone of the reaction mixture concurrently with the biological sample and reagents. Subjecting the first thermal zone to temperature conditions suitable for action of the lysis agent may be used to lyse cells and viral particles in the biological sample in the first thermal zone, such that nucleic acids in the biological sample are released into the reaction mixture. After lysis, the reaction mixture can then be permitted to enter a second thermal zone of the reaction vessel for amplification of the released nucleic acid, using amplification methods described herein.

The lysis buffer may comprise any suitable lysis agent, including commercially available lysis agents. Non-limiting examples of lysis agents include Tris-HCl, EDTA, detergents (e.g., Triton X-100, SDS), lysozyme, glucolase, proteinase E, viral endolysins, exolysins zymolose, lyticase, proteinase K, endolysins and exolysins from bacteriophages, endolysins from bacteriophage PM2, endolysins from the B. subtilis bacteriophage PBSX, endolysins from Lactobacillus prophages Lj928, Lj965, bacteriophage 15 Phiadh, endolysin from the Streptococcus pneumoniae bacteriophage Cp-I, bifunctional peptidoglycan lysin of Streptococcus agalactiae bacteriophage B30, endolysins and exolysins from prophage bacteria, endolysins from Listeria bacteriophages, holin-endolysin, cell 20 lysis genes, holWMY Staphylococcus wameri M phage varphiWMY, Iy5WMY of the Staphylococcus wameri M phage varphiWMY, Tween 20, PEG, KOH, NaCl, and combinations thereof. An example of a lysis buffer is sodium hydroxide (NaOH). In some embodiments, the biological sample is not treated with a detergent. For example, the lysis buffer may not contain any detergent.

The lysis buffer may have a pH from about 7 to 14, such as from about 8 to 13, from about 9 to 12, from about 10 to 11. For example, the lysis buffer may have a pH of about 7, 7.5, 8, 8.5, 9, 9.5, 10, 10.5, 11, 11.5, 12, 12.5, 13, 13.5, or 14.

In any of the various aspects of the present disclosure, when the biological sample is mixed with a lysis buffer to obtain a mixture, a ratio of the biological sample to the lysis buffer may be between about 5:1 (wt/vol) to about 1:10 (wt/vol), e.g., from about 1:1 (wt/vol) to about 1:10 (wt/vol). For example, the ratio of the biological sample to the lysis buffer may be about 5:1 (wt/vol), about 4:1 (wt/vol), about 3:1 (wt/vol), about 2:1 (wt/vol), about 1:1 (wt/vol), about 1:2 (wt/vol), about 1:3 (wt/vol), about 1:4 (wt/vol), about 1:5 (wt/vol), about 1:6 (wt/vol), about 1:7 (wt/vol), about 1:8 (wt/vol), about 1:9 (wt/vol), or about 1:10 (wt/vol).

In some embodiments, the reaction vessel contains reagents necessary for nucleic acid amplification. For example, the reagents include those necessary for reverse transcription amplification (e.g., reverse transcriptase) or DNA amplification (e.g., DNA polymerase).

Any type of nucleic acid amplification reaction may be used to amplify a target nucleic acid and generate an amplified product. Moreover, amplification of a nucleic acid may linear, exponential, or a combination thereof. Amplification may be emulsion based or may be non-emulsion based. Non-limiting examples of nucleic acid amplification methods include reverse transcription, primer extension, polymerase chain reaction, ligase chain reaction, helicase-dependent amplification, asymmetric amplification, rolling circle amplification, and multiple displacement amplification (MDA). In some embodiments, the amplified product may be DNA. In cases where a target RNA is amplified, DNA can be obtained by reverse transcription of the RNA and subsequent amplification of the DNA can be used to generate an amplified DNA product. The amplified DNA product may be indicative of the presence of the target RNA in the biological sample. In cases where DNA is amplified, various DNA amplification protocols may be employed. Non-limiting examples of DNA amplification methods include polymerase chain reaction (PCR), variants of PCR (e.g., real-time PCR, allele-specific PCR, assembly PCR, asymmetric PCR, digital PCR, emulsion PCR, dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, touchdown PCR), and ligase chain reaction (LCR). In some cases, DNA amplification is linear. In some cases, DNA amplification is exponential. In some cases, DNA amplification is achieved with nested PCR, which can improve sensitivity of detecting amplified DNA products. In some embodiments, amplification as described herein may refer to a primer extension reaction.

In various aspects, nucleic acid amplification reactions described herein may be conducted in parallel. In general, parallel amplification reactions are amplification reactions that occur in the same reaction vessel and at the same time. Parallel nucleic acid amplification reactions may be conducted, for example, by including reagents necessary for each nucleic acid amplification reaction in a reaction vessel to obtain a reaction mixture and subjecting the reaction mixture to conditions necessary for each nucleic amplification reaction. For example, reverse transcription amplification and DNA amplification may be conducted in parallel, by providing reagents necessary for both amplification methods in a reaction vessel to form to obtain a reaction mixture and subjecting the reaction mixture to conditions suitable for conducting both amplification reactions. DNA generated from reverse transcription of the RNA may be amplified in parallel to generate an amplified DNA product. Any suitable number of nucleic acid amplification reactions may be conducted in parallel. In some cases, at least 1, 2, 3, 4, 5, 6, 7, 8, 9, 10, 11, 12, 13, 14, 15, 16, 17, 18, 19, 20, or more nucleic acid amplification reactions are conducted in parallel.

An advantage of conducting nucleic acid amplification reactions in parallel can include fast transitions between coupled nucleic acid amplification reactions. For example, a target nucleic acid (e.g., target RNA, target DNA) may be extracted or released from a biological sample during heating phases of parallel nucleic acid amplification. In the case of a target RNA, for example, the biological sample comprising the target RNA can be heated and the target RNA released from the biological sample. The released target RNA can immediately begin reverse transcription (via reverse transcription amplification) to produce complementary DNA. The complementary DNA can then be immediately amplified, often on the order of seconds. Short times between release of a target RNA from a biological sample and reverse transcription of the target RNA to complementary DNA may help minimize the effects of inhibitors in the biological sample that may impede reverse transcription and/or DNA amplification.

In any of the various aspects, primer sets directed to a target nucleic acid may be utilized to conduct nucleic acid amplification reaction. For example, the reagents necessary for conducting the nucleic acid amplification reaction may comprise one or more primer sets. Primer sets generally comprise one or more primers. For example, a primer set may comprise about 1, 2, 3, 4, 5, 6, 7, 8, 9, 10, or more primers. In some cases, a primer set or may comprise primers directed to different amplified products or different nucleic acid amplification reactions. For example, a primer set may comprise a first primer necessary to generate a first strand of nucleic acid product that is complementary to at least a portion of the target nucleic acid and a second primer complementary to the nucleic acid strand product necessary to generate a second strand of nucleic acid product that is complementary to at least a portion of the first strand of nucleic acid product.

For example, a primer set may be directed to a target RNA. The primer set may comprise a first primer that can be used to generate a first strand of nucleic acid product that is complementary to at least a portion the target RNA. In the case of a reverse transcription reaction, the first strand of nucleic acid product may be DNA. The primer set may also comprise a second primer that can be used to generate a second strand of nucleic acid product that is complementary to at least a portion of the first strand of nucleic acid product. In the case of a reverse transcription reaction conducted in parallel with DNA amplification, the second strand of nucleic acid product may be a strand of nucleic acid (e.g., DNA) product that is complementary to a strand of DNA generated from an RNA template.

Where desired, any suitable number of primer sets may be used. For example, about 1, 2, 3, 4, 5, 6, 7, 8, 9, 10, or more primer sets may be used. Where multiple primer sets are used, one or more primer sets may each correspond to a particular nucleic acid amplification reaction or amplified product.

In some embodiments, each primer set of the one or more primer sets is capable of specifically binding to a target nucleic acid sequence from a microbial genome or a microbial transcriptome, or a variant thereof. In some cases, the microbial genome is a Salmonella genome. In some cases, the microbial transcriptome is a Salmonella transcriptome. The target nucleic acid may be a target RNA or a target DNA. For example, the target nucleic acid may be a target DNA sequence from the Salmonella genome. For example, the target nucleic acid may be a target RNA sequence from the Salmonella transcriptome. For example, the target nucleic acid may be other type of target RNA sequences, such as rRNA, tRNA, nRNA, siRNA, snRNA, snoRNA, scaRNA, microRNA, dsRNA, but not limited thereto.

In cases where the target nucleic acid is a target RNA, it may be any type of RNA, including types of RNA as described elsewhere herein. In some embodiments, the target RNA is an mRNA. In some embodiments, the target RNA is a Salmonella mRNA. In some embodiments, the target RNA may be selected from an mRNA with a high expression abundance in Salmonella. In some embodiments, the target RNA is selected from the group consisting of ttr mRNA, invA mRNA, prgK mRNA, RpoS mRNA, RpoD mRNA, and combination thereof. In some embodiments, the target RNA is invA mRNA.

In some embodiments, the one or more primer set includes a primer set capable of specifically binding to invA mRNA. For example, the one or more primer set may include a primer set comprising a forward primer as depicted in SEQ ID NO: 1 (TGCTCGTTTACGACCTGAATTA) and a reverse primer depicted in SEQ ID NO: 2 (ACACCAATATCGCCAGTACG). Alternatively or additionally, the one or more primer set may include a primer set comprising a forward primer as depicted in SEQ ID NO: 4 (TCGTTTACGACCTGAATTAC) and a reverse primer depicted in SEQ ID NO: 5 (TAGAACGACCCCATAAACA).

In cases where the target nucleic acid is a target DNA, it may be any type of DNA, including types of DNA as described elsewhere herein. In some embodiments, the target DNA is a genome DNA. In some embodiments, the target DNA is a Salmonella genome DNA. In some embodiments, the target DNA may be selected from a gene specific to and/or conservative among Salmonella. In some embodiments, the target DNA is selected from the group consisting of ttr gene, invA gene, prgK gene, RpoS gene, RpoD gene, and combination thereof. In some embodiments, the target DNA is ttr gene.

In some embodiments, the one or more primer set includes a primer set capable of specifically binding to ttr gene. For example, the one or more primer set may include a primer set comprising a forward primer as depicted in SEQ ID NO: 7 (CTCACCAGGAGATTACAACATGG) and a reverse primer depicted in SEQ ID NO: 8 (AGCTCAGACCAAAAGTGACCATC).

In some embodiments, the one or more primer sets may comprise a primer set capable of specifically binding to a target nucleic acid sequence from a bacterial genome or a variant thereof. In some embodiments, the one or more primer sets may comprise a primer set capable of specifically binding to a target nucleic acid sequence from a bacterial transcriptome or a variant thereof. In some embodiments, the one or more primer sets may comprise both a primer set capable of specifically binding to a target nucleic acid sequence from a bacterial genome or a variant thereof and a primer set capable of specifically binding to a target nucleic acid sequence from a bacterial transcriptome or a variant thereof. The bacterial genome may be a Salmonella genome. The bacterial transcriptome may be a Salmonella transcriptome. For example, the one or more primer sets may comprise a primer set capable of specifically binding to ttr gene from the Salmonella genome, or a variant thereof. For example, the one or more primer sets may comprise a primer set capable of specifically binding to invA mRNA from the Salmonella transcriptome, or a variant thereof. For example, the one or more primer sets may comprise both a primer set capable of specifically binding to ttr gene from the Salmonella genome, or a variant thereof and a primer set capable of specifically binding to invA mRNA from the Salmonella transcriptome, or a variant thereof.

In any of the various aspects of the present disclosure, the reagent may comprise a forward primer. The reagent may comprise any amount of the forward primer suitable for conducting the nucleic acid amplification. For example, the reagent may comprise less than 0.01 μM, 0.01 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 1.6 μM, 1.7 μM, 1.8 μM, 1.9 μM, 2.0 μM, 2.5 μM, 3.0 μM, 3.5 μM, 4.0 μM, 4.5 μM, 5.0 μM, 6.0 μM, 7.0 μM, 8.0 μM, 9.0 μM, 10.0 μM, 11.0 μM, 12.0 μM, 13.0 μM, 14.0 μM, 15.0 μM, more than 15.0 μM forward primer, or any concentration range between the aforesaid numeric values, such as about 0.01 to 10.0 μM, 0.05 to 5.0 μM, 0.05 to 4.0 μM, 0.05 to 3.0 μM, 0.05 to 2.0 μM, 0.05 to 1.0 μM, 0.05 to 0.5 μM, 0.1 to 5.0 μM, 0.1 to 4.0 μM, 0.1 to 3.0 μM, 0.1 to 2.0 μM, 0.1 to 1.0 μM, 0.1 to 0.9 μM, 0.1 to 0.8 μM, 0.1 to 0.7 μM, 0.1 to 0.6 μM, 0.1 to 0.5 μM forward primer. In one aspect, the reagent may comprise about 0.1 to 1.0 μM forward primer.

In any of the various aspects of the present disclosure, the reagent may comprise a reverse primer. The reagent may comprise any amount of the reverse primer suitable for conducting the nucleic acid amplification. For example, the reagent may comprise less than 0.01 μM, 0.01 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 1.6 μM, 1.7 μM, 1.8 μM, 1.9 μM, 2.0 μM, 2.5 μM, 3.0 μM, 3.5 μM, 4.0 μM, 4.5 μM, 5.0 μM, 6.0 μM, 7.0 μM, 8.0 μM, 9.0 μM, 10.0 μM, 11.0 μM, 12.0 μM, 13.0 μM, 14.0 μM, 15.0 μM, more than 15.0 μM reverse primer, or any concentration range between the aforesaid numeric values, such as about 0.01 to 10.0 μM, 0.05 to 5.0 μM, 0.05 to 4.0 μM, 0.05 to 3.0 μM, 0.05 to 2.0 μM, 0.05 to 1.0 μM, 0.05 to 0.5 μM, 0.1 to 5.0 μM, 0.1 to 4.0 μM, 0.1 to 3.0 μM, 0.1 to 2.0 μM, 0.1 to 1.0 μM, 0.1 to 0.9 μM, 0.1 to 0.8 μM, 0.1 to 0.7 μM, 0.1 to 0.6 μM, 0.1 to 0.5 μM reverse primer. In one aspect, the reagent may comprise about 0.1 to 1.0 μM reverse primer.

In some embodiments, a DNA polymerase is used. Any suitable DNA polymerase may be used, including commercially available DNA polymerases. A DNA polymerase generally refers to an enzyme that is capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases.

In some embodiments, the reagent necessary for conducting the nucleic acid amplification may comprise a reverse transcriptase. In some embodiments, the reagent as described herein may comprise a reverse transcriptase. For example, any suitable reverse transcriptase may be used. A reverse transcriptase generally refers to an enzyme that is capable of incorporating nucleotides to a strand of DNA, when bound to an RNA template. Non-limiting examples of reverse transcriptases include HIV-1 reverse transcriptase, M-MLV reverse transcriptase, AMV reverse transcriptase, telomerase reverse transcriptase, and variants, modified products and derivatives thereof. Many DNA and RNA polymerases disclosed herein or otherwise known in the art are capable of utilizing nucleotide analogs in a template-directed primer extension reaction. A wide variety of nucleotide analogs are known in the art. Non-limiting examples of nucleotide analogs include ribonuclotide and deoxyribonucleotide analogs. Typically, such analogs include, adenosine, thymidine, guanosine, cytidine, uracil, and analogs of these bases. The analogs may comprise nucleoside triphosphates, or may include additional phosphate groups, e.g., tetraphosphates, pentaphosphates, hexaphosphates, heptaphosphates, or greater. Examples of some of these analogs are described, for example, in Published U.S. Patent Application Nos. 2003-0124576 and 2007-0072196, as well as U.S. Pat. Nos. 7,223,541 and 7,052,839, the full disclosures of which are incorporated herein by reference in their entirety for all purposes.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration.

In some cases, prior to the primer extension reaction, the target nucleic acid molecule may be subject to one or more denaturing conditions. The one or more denaturing conditions may be selected from a denaturing temperature profile and a denaturing agent.

In some cases, prior to the primer extension reaction, the biological sample is preheated at a preheating temperature between about 90° C. to 100° C. for a preheating duration no longer than about 10 minutes. In some embodiments, the preheating duration is no longer than 1 minute.

Denaturation temperatures may vary depending upon, for example, the particular biological sample analyzed, the particular source of target nucleic acid (e.g., viral particle, bacteria) in the biological sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

Denaturation durations may vary depending upon, for example, the particular biological sample analyzed, the particular source of target nucleic acid (e.g., viral particle, bacteria) in the biological sample, the reagents used, and/or the desired reaction conditions. For example, a denaturation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, a denaturation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

Elongation temperatures may vary depending upon, for example, the particular biological sample analyzed, the particular source of target nucleic acid (e.g., viral particle, bacteria) in the biological sample, the reagents used, and/or the desired reaction conditions. For example, an elongation temperature may be from about 30° C. to about 80° C. In some examples, an elongation temperature may be from about 35° C. to about 72° C. In some examples, an elongation temperature may be from about 45° C. to about 65° C. In some examples, an elongation temperature may be from about 35° C. to about 65° C. In some examples, an elongation temperature may be from about 40° C. to about 60° C. In some examples, an elongation temperature may be from about 50° C. to about 60° C. In still other examples, an elongation temperature may be about 35°, 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., or 80° C.

Elongation durations may vary depending upon, for example, the particular biological sample analyzed, the particular source of target nucleic acid (e.g., viral particle, bacteria) in the biological sample, the reagents used, and/or the desired reaction conditions. For example, an elongation duration may be less than or equal to 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. For example, an elongation duration may be no more than 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second.

In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) necessary to obtain a detectable amplified product (e.g., a detectable amount of amplified DNA product that is indicative of the presence of a target RNA in a biological sample). For example, the number of cycles necessary to obtain a detectable amplified product (e.g., a detectable amount of DNA product that is indicative of the presence of a target RNA in a biological sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target RNA in a biological sample) may be obtained at a cycle threshold value (Ct) of less than 100, 75, 70, 65, 60, 55, 50, 45, 40, 35, 30, 25, 20, 15, 10, or 5.

The time for which amplification yields a detectable amount of amplified product indicative of the presence of a target nucleic acid amplified can vary depending upon the biological sample from which the target nucleic acid was obtained, the particular nucleic acid amplification reactions to be conducted, and the particular number of cycles of amplification reaction desired. For example, amplification of a target nucleic acid may yield a detectable amount of amplified product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a target RNA may yield a detectable amount of amplified DNA product indicative to the presence of the target RNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, a reaction mixture may be subjected to a plurality of series of primer extension reactions. An individual series of the plurality may comprise multiple cycles of a particular primer extension reaction, characterized, for example, by particular denaturation and elongation conditions as described elsewhere herein. Generally, each individual series differs from at least one other individual series in the plurality with respect to, for example, a denaturation condition and/or elongation condition. An individual series may differ from another individual series in a plurality of series, for example, with respect to any one, two, three, four, or all five of ramping rate, denaturing temperature, denaturing duration, elongation temperature, and elongation duration. Moreover, a plurality of series may comprise any number of individual series such as, for example, at least about or about 2, 3, 4, 5, 6, 7, 8, 9, 10, or more individual series.

For example, a plurality of series of primer extension reactions may comprise a first series and a second series. The first series, for example, may comprise more than ten cycles of a primer extension reaction, where each cycle of the first series comprises (i) incubating a reaction mixture at about 92° C. to about 95° C. for no more than 30 seconds followed by (ii) incubating the reaction mixture at about 35° C. to about 65° C. for no more than about one minute. The second series, for example, may comprise more than ten cycles of a primer extension reaction, where each cycle of the second series comprises (i) incubating the reaction mixture at about 92° C. to about 95° C. for no more than 30 seconds followed by (ii) incubating the reaction mixture at about 40° C. to about 60° C. for no more than about 1 minute. In this particular example, the first and second series differ in their elongation temperature condition. The example, however, is not meant to be limiting as any combination of different elongation and denaturing conditions may be used.

Any suitable number of cycles may be conducted in each series of the plurality of series of primer extension reactions. For example, the number of cycles conducted in each series of the plurality of series of primer extension reactions may be less than about 100, 90, 80, 70, 60, 50, 40, 30, 20, 10, or 5 cycles.

An advantage of conducting a plurality of series of primer extension reaction may be that, when compared to a single series of primer extension reactions under comparable denaturing and elongation conditions, the plurality of series approach yields a detectable amount of amplified product that is indicative of the presence of a target nucleic acid in a biological sample with a lower cycle threshold value. Use of a plurality of series of primer extension reactions may reduce such cycle threshold values by at least about or about 1%, 5%, 10%, 15%, 20%, 25%, 30%, 35%, 40%, 45%, 50%, 55%, 60%, 65%, 70%, 75%, 80%, 85%, 90%, or 95% when compared to a single series under comparable denaturing and elongation conditions.

In some embodiments, the ramping time (i.e., the time the thermal cycler takes to transition from one temperature to another) and/or ramping rate can be important factors in amplification. For example, the temperature and time for which amplification yields a detectable amount of amplified product indicative of the presence of a target nucleic acid can vary depending upon the ramping rate and/or ramping time. The ramping rate can impact the temperature(s) and time(s) used for amplification.

In some cases, the ramping time and/or ramping rate can be different between cycles. In some situations, however, the ramping time and/or ramping rate between cycles can be the same. The ramping time and/or ramping rate can be adjusted based on the sample(s) that are being processed.

In some situations, the ramping time between different temperatures can be determined, for example, based on the nature of the sample and the reaction conditions. The exact temperature and incubation time can also be determined based on the nature of the sample and the reaction conditions. In some embodiments, a single sample can be processed (e.g., subjected to amplification conditions) multiple times using multiple thermal cycles, with each thermal cycle differing for example by the ramping time, temperature, and/or incubation time. The best or optimum thermal cycle can then be chosen for that particular sample. This provides a robust and efficient method of tailoring the thermal cycles to the specific sample or combination of samples being tested.

In some embodiments, a target nucleic acid may be subjected to a denaturing condition prior to initiation of a primer extension reaction. In the case of a plurality of series of primer extension reactions, the target nucleic acid may be subjected to a denaturing condition prior to executing the plurality of series or may be subjected to a denaturing condition between series of the plurality. For example, the target nucleic acid may be subjected to a denaturing condition between a first series and a second series of a plurality of series. Non-limiting examples of such denaturing conditions include a denaturing temperature profile (e.g., one or more denaturing temperatures) and a denaturing agent.

In some embodiments, a biological sample may be preheated prior to conducting a primer extension reaction. The temperature (e.g., a preheating temperature) at which and duration (e.g., a preheating duration) for which a biological sample is preheated may vary depending upon, for example, the particular biological sample being analyzed. In some examples, a biological sample may be preheated for no more than about 60 minutes, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 45 seconds, 30 seconds, 20 seconds, 15 seconds, 10 seconds, or 5 seconds. In some examples, a biological sample may be preheated at a temperature from about 80° C. to about 110° C. In some examples, a biological sample may be preheated at a temperature from about 90° C. to about 100° C. In some examples, a biological sample may be preheated at a temperature from about 90° C. to about 97° C. In some examples, a biological sample may be preheated at a temperature from about 92° C. to about 95° C. In still other examples, a biological sample may be preheated at a temperature of about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

In some embodiments, reagents necessary for conducting nucleic acid amplification, including reagents necessary for conduction of parallel nucleic acid amplification may also include a reporter agent. The reporter agent may yield a detectable signal. The detectable signal may indicate whether the amplified product is present. For example, the presence or absence of the detectable signal may be indicative of the presence of an amplified product. The intensity of the detectable signal may be proportional to the amount of amplified product. In some cases, where amplified product is generated of a different type of nucleic acid than the target nucleic acid initially amplified, the intensity of the detectable signal may be proportional to the amount of target nucleic acid initially amplified. For example, in the case of amplifying a target RNA via parallel reverse transcription and amplification of the DNA obtained from reverse transcription, reagents necessary for both reactions may also comprise a reporter agent may yield a detectable signal that is indicative of the presence of the amplified DNA product and/or the target RNA amplified. The intensity of the detectable signal may be proportional to the amount of the amplified DNA product and/or the original target RNA amplified. The use of a reporter agent also enables real-time amplification methods, including real-time PCR for DNA amplification.

Reporter agents may be linked with nucleic acids, including amplified products, by covalent or non-covalent interactions. Non-limiting examples of non-covalent interactions include ionic interactions, Van der Waals forces, hydrophobic interactions, hydrogen bonding, and combinations thereof. In some embodiments, reporter agents may bind to initial reactants and changes in reporter agent levels may be used to detect amplified product. In some embodiments, reporter agents may only be detectable (or non-detectable) as nucleic acid amplification progresses. In some embodiments, an optically-active dye (e.g., a fluorescent dye) may be used as may be used as a reporter agent. Non-limiting examples of dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, VIC, NED, PET, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein (TET), 5 and/or 6-carboxy fluorescein (FAM), 5- (or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 5 and/or 6 carboxy tetramethylrhodamine (TAMRA), 6-carboxy-4′,5′-dichloro-2′,7′-dimethoxyfluorescein (JOE), 6-carboxy-2′,4,4′,5′,7,7′-hexachlorofluorescein (HEX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some embodiments, a reporter agent may be a sequence-specific oligonucleotide probe that is optically active when hybridized with an amplified product. Due to sequence-specific binding of the probe to the amplified product, use of oligonucleotide probes can increase specificity and sensitivity of detection. A probe may be linked to any of the optically-active reporter agents (e.g., dyes) described herein and may also include a quencher capable of blocking the optical activity of an associated dye. Non-limiting examples of probes that may be useful used as reporter agents include TaqMan probes, TaqMan Tamara probes, TaqMan MGB probes, or Lion probes.

In some embodiments, a reporter agent may be a sequence specific oligonucleotide probe having blocked optical activity upon hybridization with an amplification product. In some embodiments, the oligonucleotide probe becomes optically active upon its breakdown. For example, the reporter agent may be an oliognucleotide probe that includes an optically-active dye (e.g., fluorescent dye) and a quencher positioned adjacently on the probe. The close proximity of the dye with the quencher can block the optical activity of the dye. The probe may bind to a target sequence to be amplified. Upon the breakdown of the probe with the exonuclease activity of a DNA polymerase during amplification, the quencher and dye are separated, and the free dye regains its optical activity that can subsequently be detected. The oligonucleotide probe may be an RNA oligonucleotide probe or a DNA oligonucleotide probe.

In some embodiments, a reporter agent may be a molecular beacon. A molecular beacon includes, for example, a quencher linked at one end of an oligonucleotide in a hairpin conformation. At the other end of the oligonucleotide is an optically active dye, such as, for example, a fluorescent dye. In the hairpin configuration, the optically-active dye and quencher are brought in close enough proximity such that the quencher is capable of blocking the optical activity of the dye. Upon hybridizing with amplified product, however, the oligonucleotide assumes a linear conformation and hybridizes with a target sequence on the amplified product. Linearization of the oligonucleotide results in separation of the optically-active dye and quencher, such that the optical activity is restored and can be detected. The sequence specificity of the molecular beacon for a target sequence on the amplified product can improve specificity and sensitivity of detection.

In some embodiments, an oligonucleotide probe or a molecular beacon as describe herein may be a sequence specific oligonucleotide probe or a sequence specific molecular beacon. In some embodiments, the oligonucleotide probe or the molecular beacon as describe herein hybridizes with a region on the target nucleic acid between the primers in the primer set capable of specifically binding to the target nucleic acid. When the primer set is used for amplification, an amplified product corresponding to the region on the target nucleic acid between the primers (including the primers) is produced. Therefore, the oligonucleotide probe or the molecular beacon as describe herein may hybridize with the amplified product. For example, the oligonucleotide probe or the molecular beacon as describe herein may hybridize with the amplified product produced from the amplification of the target nucleic acid.

The target nucleic acid may be a target RNA or a target DNA. In cases where the target nucleic acid is a target RNA, it may be any type of RNA, including types of RNA as described elsewhere herein. In some embodiments, the target RNA is an mRNA. In some embodiments, the target RNA is a Salmonella mRNA. In some embodiments, the target RNA may be selected from an mRNA with a high expression abundance in Salmonella. In some embodiments, the target RNA is selected from the group consisting of ttr mRNA, invA mRNA, prgK mRNA, RpoS mRNA, RpoD mRNA, and combination thereof. In some embodiments, the target RNA is invA mRNA. In cases where the target nucleic acid is a target DNA, it may be any type of DNA, including types of DNA as described elsewhere herein. In some embodiments, the target DNA is a genome DNA. In some embodiments, the target DNA is a Salmonella genome DNA. In some embodiments, the target DNA may be selected from a gene specific to and/or conservative among Salmonella. In some embodiments, the target DNA is selected from the group consisting of ttr gene, invA gene, prgK gene, RpoS gene, RpoD gene, and combination thereof. In some embodiments, the target DNA is ttr gene.

In some embodiments, the oligonucleotide probe or a molecular beacon as describe herein may hybridize with the amplified product produce by the amplification of invA mRNA. For example, the oligonucleotide probe or a molecular beacon may comprise a nucleic acid sequence as depicted in SEQ ID NO: 3 (TCTGGTTGATTTCCTGATCGCACTGA) and/or in SEQ ID NO: 6 (CTGGTTGATTTCCTGATCGCACT). Alternatively or additionally, the oligonucleotide probe or a molecular beacon as describe herein may hybridize with the amplified product produce by the amplification of ttr gene. For example, the oligonucleotide probe or a molecular beacon may comprise a nucleic acid sequence as depicted in SEQ ID NO: 9 (CACCGACGGCGAGACCGACTTT).

In any of the various aspects of the present disclosure, the reagent may comprise one or more oligonucleotide probes or molecular beacons. The reagent may comprise any amount of the one or more oligonucleotide probes or molecular beacons suitable for conducting the nucleic acid amplification. For example, the reagent may comprise less than 0.01 μM, 0.01 μM, 0.05 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 1.6 μM, 1.7 μM, 1.8 μM, 1.9 μM, 2.0 μM, 2.5 μM, 3.0 μM, 3.5 μM, 4.0 μM, 4.5 μM, 5.0 μM, 6.0 μM, 7.0 μM, 8.0 μM, 9.0 μM, 10.0 μM, 11.0 μM, 12.0 μM, 13.0 μM, 14.0 μM, 15.0 μM, more than 15.0 μM one or more oligonucleotide probes or molecular beacons, or any concentration range between the aforesaid numeric values, such as about 0.01 to 10.0 μM, 0.05 to 5.0 μM, 0.05 to 4.0 μM, 0.05 to 3.0 μM, 0.05 to 2.0 μM, 0.05 to 1.0 μM, 0.05 to 0.5 μM, 0.1 to 5.0 μM, 0.1 to 4.0 μM, 0.1 to 3.0 μM, 0.1 to 2.0 μM, 0.1 to 1.0 μM, 0.1 to 0.9 μM, 0.1 to 0.8 μM, 0.1 to 0.7 μM, 0.1 to 0.6 μM, 0.1 to 0.5 μM one or more oligonucleotide probes or molecular beacons. In one aspect, the reagent may comprise about 0.1 to 0.5 μM one or more oligonucleotide probes or molecular beacons.

In some embodiments, a reporter agent may be a radioactive species. Non-limiting examples of radioactive species include ¹⁴C, ¹²³I, ¹²⁴I, ¹²⁵I, ¹³¹I, Tc99m, ³⁵S, or ³H.

In some embodiments, a reporter agent may be an enzyme that is capable of generating a detectable signal. Detectable signal may be produced by activity of the enzyme with its substrate or a particular substrate in the case the enzyme has multiple substrates. Non-limiting examples of enzymes that may be used as reporter agents include alkaline phosphatase, horseradish peroxidase, I²-galactosidase, alkaline phosphatase, β-galactosidase, acetylcholinesterase, and luciferase.

In amplification reactions conducted in parallel, a plurality of reporter agents may be employed to detect a plurality of amplified products. Each of the plurality of reporter agents may produce a detectable signal, while the plurality of detectable signals produced by the plurality of reporter agents is distinct among one another. Each of the plurality of detectable signals may be indicative of the presence or absence of the corresponding amplified product. The intensity of each of the plurality of detectable signals may be proportional to the amount of the corresponding amplified product. For example, the detectable signal corresponding to one amplified product may be FAM fluorescence, while the detectable signal corresponding to another amplified product may be ROX fluorescence. Detection of distinct detectable signals in parallel may allow comparison of the presence and/or amount among different amplified products. Alternatively or additionally, detection of distinct detectable signals in parallel may allow determination of the amount of the amplified product(s) relative to an internal reference.

In any of the various aspect of the present disclosure, the reagent may further comprise MgCl₂. The reagent may comprise any amount of MgCl₂ suitable for conducting the nucleic acid amplification. For example, the reagent may comprise about less than 0.01 mM, 0.01 mM, 0.05 mM, 0.06 mM, 0.07 mM, 0.08 mM, 0.09 mM, 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.5 mM, 3.0 mM, 3.5 mM, 4.0 mM, 4.5 mM, 5.0 mM, 6.0 mM, 7.0 mM, 8.0 mM, 9.0 mM, 10.0 mM, 11.0 mM, 12.0 mM, 13.0 mM, 14.0 mM, 15.0 mM, more than 15.0 mM MgCl₂, or any concentration range between the aforesaid numeric values, such as about 0.01 to 15.0 mM, 0.05 to 14.0 mM, 0.1 to 13.0 mM, 0.2 to 12.0 mM, 0.3 to 11.0 mM, 0.4 to 10.0 mM, 0.5 to 9.0 mM, 0.6 to 8.0 mM, 0.7 to 7.0 mM, 0.8 to 6.0 mM, 0.9 to 5.0 mM, 1.0 to 4.0 mM, 1.1 to 3.0 mM, 1.2 to 2.5 mM, 1.3 to 2.0 mM, or 1.4 to 1.6 mM MgCl₂. In one aspect, the reagent may comprise about 1.5 mM MgCl₂.

In any of the various aspect of the present disclosure, the reagent may further comprise dNTPs. The reagent may comprise any amount of dNTPs suitable for conducting the nucleic acid amplification. For example, the reagent may comprise about less than 0.01 mM, 0.01 mM, 0.05 mM, 0.06 mM, 0.07 mM, 0.08 mM, 0.09 mM, 0.1 mM, 0.2 mM, 0.3 mM, 0.4 mM, 0.5 mM, 0.6 mM, 0.7 mM, 0.8 mM, 0.9 mM, 1.0 mM, 1.1 mM, 1.2 mM, 1.3 mM, 1.4 mM, 1.5 mM, 1.6 mM, 1.7 mM, 1.8 mM, 1.9 mM, 2.0 mM, 2.5 mM, 3.0 mM, 3.5 mM, 4.0 mM, 4.5 mM, 5.0 mM, 6.0 mM, 7.0 mM, 8.0 mM, 9.0 mM, 10.0 mM, 11.0 mM, 12.0 mM, 13.0 mM, 14.0 mM, 15.0 mM, more than 15.0 mM dNTPs, or any concentration range between the aforesaid numeric values, such as about 0.01 to 10.0 mM, 0.05 to 5.0 mM, 0.05 to 4.0 mM, 0.05 to 3.0 mM, 0.05 to 2.0 mM, 0.05 to 1.0 mM, 0.05 to 0.5 mM, 0.1 to 5.0 mM, 0.1 to 4.0 mM, 0.1 to 3.0 mM, 0.1 to 2.0 mM, 0.1 to 1.0 mM, 0.1 to 0.9 mM, 0.1 to 0.8 mM, 0.1 to 0.7 mM, 0.1 to 0.6 mM, 0.1 to 0.5 mM dNTPs. In one aspect, the reagent may comprise about 1.5 mM dNTPs.

In various aspects, amplified product (e.g., amplified DNA product, amplified RNA product) may be detected. Detection of amplified product, including amplified DNA, may be accomplished with any suitable detection method. The particular type of detection method used may depend, for example, on the particular amplified product, the type of reaction vessel used for amplification, other reagents in a reaction mixture, whether or not a reporter agent was included in a reaction mixture, and if a reporter agent was used, the particular type of reporter agent use. Non-limiting examples of detection methods include optical detection, spectroscopic detection, electrostatic detection, electrochemical detection, and the like. Optical detection methods include, but are not limited to, fluorimetry and UV-vis light absorbance. Spectroscopic detection methods include, but are not limited to, mass spectrometry, nuclear magnetic resonance (NMR) spectroscopy, and infrared spectroscopy. Electrostatic detection methods include, but are not limited to, gel based techniques, such as, for example, gel electrophoresis. Electrochemical detection methods include, but are not limited to, electrochemical detection of amplified product after high-performance liquid chromatography separation of the amplified products.

In some embodiments, the amplified product may be detected in the primer extension reaction. In some embodiments, the amplified product may be detected in a plurality of series of the primer extension reaction. In some embodiments, the amplified product may be detected in each individual series of the plurality of series of the primer extension reaction. In some embodiments, the amplified product may be detected in some individual series, but not other individual series of the plurality of series of the primer extension reaction. For example, the plurality of series of the primer extension reaction may comprise a first series and a second series. The amplified product may be detected not in the first series, but in the second series. Alternatively, the amplified product may be detected not in the second series, but in the first series. Alternatively, the amplified product may be detected in both the first series and the second series, or in neither the first series nor the second series.

In some embodiments, the detectable signal produced by the reporter agent may be detected in the primer extension reaction. In some embodiments, the detectable signal produced by the reporter agent may be detected in a plurality of series of the primer extension reaction. In some embodiments, the detectable signal produced by the reporter agent may be detected in each individual series of the plurality of series of the primer extension reaction. In some embodiments, the detectable signal produced by the reporter agent may be detected in some individual series, but not other individual series of the plurality of series of the primer extension reaction. For example, the plurality of series of the primer extension reaction may comprise a first series and a second series. The detectable signal produced by the reporter agent may be detected not in the first series, but in the second series. Alternatively, the detectable signal produced by the reporter agent may be detected not in the second series, but in the first series. Alternatively, the detectable signal produced by the reporter agent may be detected in both the first series and the second series, or in neither the first series nor the second series.

In any of the various aspects, the time required to complete the elements of a method may vary depending upon the particular steps of the method. For example, an amount of time for completing the elements of a method may be from about 5 minutes to about 120 minutes. In other examples, an amount of time for completing the elements of a method may be from about 5 minutes to about 60 minutes. In other examples, an amount of time for completing the elements of a method may be from about 5 minutes to about 30 minutes. In other examples, an amount of time for completing the elements of a method may be less than or equal to 120 minutes, less than or equal to 90 minutes, less than or equal to 75 minutes, less than or equal to 60 minutes, less than or equal to 45 minutes, less than or equal to 40 minutes, less than or equal to 35 minutes, less than or equal to 30 minutes, less than or equal to 25 minutes, less than or equal to 20 minutes, less than or equal to 15 minutes, less than or equal to 10 minutes, or less than or equal to 5 minutes.

In some embodiments, information regarding the presence of and/or an amount of amplified product (e.g., amplified DNA product) may be outputted to a recipient. Information regarding amplified product may be outputted via various approaches. In some embodiments, such information may be provided verbally to a recipient. In some embodiments, such information may be provided in a report. A report may include any number of desired elements, with non-limiting examples that include information regarding the subject (e.g., sex, age, race, health status, etc.) raw data, processed data (e.g., graphical displays (e.g., figures, charts, data tables, data summaries), determined cycle threshold values, calculation of starting amount of target polynucleotide), conclusions about the presence of the target nucleic acid, diagnosis information, prognosis information, disease information, and the like, and combinations thereof. The report may be provided as a printed report (e.g., a hard copy) or may be provided as an electronic report. In some embodiments, including cases where an electronic report is provided, such information may be outputted via an electronic display (e.g., an electronic display screen), such as a monitor or television, a screen operatively linked with a unit used to obtain the amplified product, a tablet computer screen, a mobile device screen, and the like. Both printed and electronic reports may be stored in files or in databases, respectively, such that they are accessible for comparison with future reports.

Moreover, a report may be transmitted to the recipient at a local or remote location using any suitable communication medium including, for example, a network connection, a wireless connection, or an internet connection. In some embodiments, a report can be sent to a recipient's device, such as a personal computer, phone, tablet, or other device. The report may be viewed online, saved on the recipient's device, or printed. A report can also be transmitted by any other approach for transmitting information, with non-limiting examples that include mailing a hard-copy report for reception and/or for review by a recipient.

Moreover, such information may be outputted to various types of recipients. Non-limiting examples of such recipients include the subject from which the biological sample was obtained, a physician, a physician treating the subject, a clinical monitor for a clinical trial, a nurse, a researcher, a laboratory technician, a representative of a pharmaceutical company, a health care company, a biotechnology company, a hospital, a human aid organization, a health care manager, an electronic system (e.g., one or more computers and/or one or more computer servers storing, for example, a subject's medical records), a public health worker, other medical personnel, and other medical facilities.

In various aspects, the present disclosure further provides computer-assisted methods and systems for performing the methods as described herein. In one aspect among the various aspects, the present disclosure provides a computer-assisted method for detecting a target nucleic acid molecule in a biological sample. The computer-assisted method may comprise: (a) an inputting step for receiving a request from a user to process the biological sample to detect the target nucleic acid molecule; (b) a mixing step for mixing the biological sample with a lysis buffer to obtain a mixture; (c) an incubating step for incubating the mixture at a temperature from about 15° C. to 70° C. for a period of time of no more than about 15 minutes; (d) an adding step for adding the mixture from (c) to a reaction vessel comprising reagents necessary for conducting nucleic acid amplification, the reagents comprising (i) a deoxyribonucleic acid (DNA) polymerase and optionally a reverse transcriptase, and (ii) a primer set for the target nucleic acid molecule, to obtain a reaction mixture; and (e) a reacting step for subjecting the reaction mixture in the reaction vessel to a plurality of series of primer extension reactions to generate amplified product(s) that is indicative of a presence of the target nucleic acid molecule in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In one aspect among the various aspects, the present disclosure provides computer-assisted method for detecting Salmonella in a biological sample. The computer-assisted method may comprise: (a) an inputting step for receiving a request from a user to process the biological sample to detect Salmonella in the biological sample; (b) a mixing step for mixing the biological sample with a lysis buffer to obtain a mixture; (c) an incubating step for incubating the mixture at an incubation temperature greater than 15° C. for an incubation time period of no more than about 15 minutes; (d) an adding step for adding the mixture from (c) to a reaction vessel comprising reagents necessary for conducting nucleic acid amplification, the reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof, to obtain a reaction mixture; and (e) a reacting step for subjecting the reaction mixture in the reaction vessel to a plurality of series of primer extension reactions to generate amplified product(s) that is indicative of a presence of the target nucleic acid(s) in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition. The biological sample may be a stool sample.

Any feature, embodiment, definition and limitation as described herein, unless apparently contradictory to the aforesaid computer-assisted methods, shall apply to the aforesaid methods.

In one aspect among the various aspects, the present disclosure provides a computer-assisted system for detecting a target nucleic acid molecule in a biological sample. The computer-assisted system may comprise: (a) an inputting means for receiving a request from a user to process the biological sample to detect the target nucleic acid molecule; (b) a mixing means for mixing the biological sample with a lysis buffer to obtain a mixture; (c) an incubating means for incubating the mixture at a temperature from about 15° C. to 70° C. for a period of time of no more than about 15 minutes; (d) an adding means for adding the mixture from (c) to a reaction vessel comprising reagents necessary for conducting nucleic acid amplification, the reagents comprising (i) a deoxyribonucleic acid (DNA) polymerase and optionally a reverse transcriptase, and (ii) a primer set for the target nucleic acid molecule, to obtain a reaction mixture; and (e) a reacting means for subjecting the reaction mixture in the reaction vessel to a plurality of series of primer extension reactions to generate amplified product(s) that is indicative of a presence of the target nucleic acid molecule in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In one aspect among the various aspects, the present disclosure provides computer-assisted system for detecting Salmonella in a biological sample. The computer-assisted system may comprise: (a) an inputting means for receiving a request from a user to process the biological sample to detect Salmonella in the biological sample; (b) a mixing means for mixing the biological sample with a lysis buffer to obtain a mixture; (c) an incubating means for incubating the mixture at an incubation temperature greater than 15° C. for an incubation time period of no more than about 15 minutes; (d) an adding means for adding the mixture from (c) to a reaction vessel comprising reagents necessary for conducting nucleic acid amplification, the reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof, to obtain a reaction mixture; and (e) a reacting means for subjecting the reaction mixture in the reaction vessel to a plurality of series of primer extension reactions to generate amplified product(s) that is indicative of a presence of the target nucleic acid(s) in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition. The biological sample may be a stool sample.

Any feature, embodiment, definition and limitation as described herein, unless apparently contradictory to the aforesaid computer-assisted systems, shall apply to the aforesaid systems.

In an aspect, the disclosure provides a system for detecting a target nucleic acid molecule in a biological sample. The system may comprise an input unit that receives a request from a user to process the biological sample to detect the target nucleic acid molecule; and one or more computer processors operatively coupled to the input unit. The one or more computer processors may be individually or collectively programmed to conduct any method as described herein. In an aspect, the one or more computer processors may be individually or collectively programmed to: (a) mix the biological sample with a lysis buffer to obtain a mixture; (b) incubate the mixture at a temperature from about 15° C. to 70° C. for a period of time of no more than about 15 minutes; (c) add the mixture from (b) to a reaction vessel comprising reagents necessary for conducting nucleic acid amplification, the reagents comprising (i) a deoxyribonucleic acid (DNA) polymerase, and (ii) a primer set for the target nucleic acid molecule, to obtain a reaction mixture; and (d) subject the reaction mixture in the reaction vessel to multiple cycles of a primer extension reaction to generate amplified product(s) that is indicative of a presence of the target nucleic acid molecule, each cycle comprising (i) incubating the reaction mixture at a denaturing temperature for a denaturing duration that is less than or equal to 60 seconds, followed by (ii) incubating the reaction mixture at an elongation temperature for an elongation duration that is less than or equal to 60 seconds, thereby amplifying the target nucleic acid molecule.

In an aspect, the present disclosure provides a system for detecting a target nucleic acid molecule in a biological sample. The system may comprise an input unit that receives a request from a user to process the biological sample to detect the target nucleic acid molecule; and one or more computer processors operatively coupled to the input unit. The one or more computer processors may be individually or collectively programmed to: (a) mix the biological sample with a lysis buffer to obtain a mixture; (b) incubate the mixture at a temperature from about 15° C. to 70° C. at a period of time of no more than about 15 minutes; (c) add the mixture from (b) to a reaction vessel comprising reagents necessary for conducting nucleic acid amplification, the reagents comprising (i) a deoxyribonucleic acid (DNA) polymerase and, in some cases, a reverse transcriptase, and (ii) a primer set for the target nucleic acid molecule, to obtain a reaction mixture; and (d) subject the reaction mixture in the reaction vessel to a plurality of series of primer extension reactions to generate amplified product(s) that is indicative of a presence of the target nucleic acid molecule in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition.

In an aspect, the present disclosure provides system for detecting Salmonella in a stool sample. The system may comprise an input unit that receives a request from a user to process said biological sample to detect Salmonella in said biological sample; and one or more computer processors operatively coupled to said input unit. The one or more computer processors may be individually or collectively programmed to: (a) mix said biological sample with a lysis buffer to obtain a mixture; (b) incubate said mixture at an incubation temperature greater than 15° C. for an incubation time period of no more than about 15 minutes; (c) add said mixture from (b) to a reaction vessel comprising reagents necessary for conducting nucleic acid amplification, said reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof, to obtain a reaction mixture; and (d) subject said reaction mixture in said reaction vessel to a plurality of series of primer extension reactions to generate amplified product(s) that is indicative of a presence of said target nucleic acid(s) in said sample, each series comprising two or more cycles of (i) incubating said reaction mixture under a denaturing condition characterized by a denaturing temperature and a denaturing duration, followed by (ii) incubating said reaction mixture under an elongation condition characterized by an elongation temperature and an elongation duration, wherein an individual series differs from at least one other individual series of said plurality with respect to said denaturing condition and/or said elongation condition. The biological sample may be a stool sample.

Any feature, embodiment, definition and limitation as described herein, unless apparently contradictory to the aforesaid systems, shall apply thereto.

The system of the present disclosure may further comprise a biological sample treatment module that mixes the biological sample with the lysis buffer to obtain the mixture. The biological sample treatment module may incubate the mixture. For example, the biological sample treatment module may comprise a heating block or an incubator that is capable of incubating the mixture of the biological sample with the lysis buffer at a temperature that is from about 10° C. to 75° C., for example, at a temperature that is from about 10° C. to 70° C., from about 15° C. to 65° C., from about 15° C. to 60° C., from about 15° C. to 55° C., from about 20° C. to 50° C., from about 20° C. to 45° C., from about 20° C. to 40° C., from about 20° C. to 35° C., from about 20° C. to 30° C., from about 20° C. to 25° C., or from about 25° C. to 30° C.

Alternatively, the biological sample treatment module may comprise a heating block or an incubator that is capable of incubating the mixture of the biological sample with the lysis buffer at a temperature that is greater than 15° C., for example, at a temperature that is greater than about 20° C., greater than about 25° C., greater than about 30° C., greater than about 35° C., greater than about 40° C., greater than about 45° C., greater than about 50° C., greater than about 55° C., greater than about 60° C., greater than about 65° C., greater than about 70° C., greater than about 75° C., greater than about 80° C., greater than about 85° C., greater than about 90° C., greater than about 91° C., greater than about 92° C., greater than about 93° C., greater than about 94° C., greater than about 95° C., greater than about 96° C., greater than about 97° C., greater than about 98° C., greater than about 99° C., greater than about 100° C., or at a temperature that is from about 15° C. to 95° C., for example, at a temperature that is from about 20° C. to 90° C., from about 25° C. to 85° C., from about 30° C. to 80° C., from about 40° C. to 70° C., from about 40° C. to 95° C., from about 45° C. to 90° C., from about 50° C. to 85° C., from about 55° C. to 80° C., from about 60° C. to 75° C., from about 65° C. to 75° C., or from about 65° C. to 70° C.

The biological sample treatment module may be able to incubate the mixture of the biological sample with the lysis buffer for a period of time that is no more than about 20 minutes. For example, the period of time in (b) may be no more than about 19 minutes, no more than about 18 minutes, no more than about 17 minutes, no more than about 16 minutes, no more than about 15 minutes, no more than about 14 minutes, no more than about 13 minutes, no more than about 12 minutes, no more than about 11 minutes, no more than about 10 minutes, no more than about 9 minutes, no more than about 8 minutes, no more than about 7 minutes, no more than about 6 minutes, no more than about 5 minutes, no more than about 4 minutes, no more than about 3 minutes, no more than about 2 minutes, no more than about 1 minute, no more than about 50 seconds, no more than about 40 seconds, no more than about 30 seconds, no more than about 20 seconds, no more than about 15 seconds, or no more than about 10 seconds.

The biological sample treatment module may be able to incubate the homogenized preparation at a temperature that is greater than about 40° C., for example, at a temperature that is greater than about 45° C., greater than about 50° C., greater than about 55° C., greater than about 60° C., greater than about 65° C., greater than about 70° C., greater than about 75° C., greater than about 80° C., greater than about 85° C., greater than about 90° C., greater than about 91° C., greater than about 92° C., greater than about 93° C., greater than about 94° C., greater than about 95° C., greater than about 96° C., greater than about 97° C., greater than about 98° C., greater than about 99° C., or greater than about 100° C.

The system of the present disclosure may further comprise an amplification module operatively coupled to the biological sample treatment module, wherein the amplification module (i) adds an amount of the mixture from the biological sample treatment module to the reaction vessel and (ii) subjects the reaction mixture in the reaction vessel to the primer extension reaction(s) to generate the amplified product that is indicative of a presence of the target nucleic acid molecule.

Alternatively, the mixture may be added to the reaction vessel manually, and the amplification module may subjects the reaction mixture in the reaction vessel to the primer extension reaction(s) to generate the amplified product that is indicative of a presence of the target nucleic acid molecule.

In some embodiments, the amplification module may automatically subjects a reaction mixture in a reaction vessel to a plurality of series of primer extension reactions to generate amplified product(s) that is indicative of a presence of target nucleic acid(s) in the sample upon recognizing information on one or more relevant parameters for conducting a primer extension reaction included in a kit for use in conjunction with the system. The information may be recognized by a recognition module in communication with the amplification module as described elsewhere herein.

The system of the present disclosure may further comprise an output module operatively coupled to the one or more computer processors, wherein the output module outputs information regarding the target nucleic acid molecule or the amplified DNA product to a recipient.

In a system of the present disclosure, prior to mixing the biological sample with a lysis buffer, the one or more computer processors may be individually or collectively programmed to suspend the biological sample in solution to obtain a homogenized preparation comprising the biological sample. In some embodiments, the one or more computer processors may be individually or collectively programmed to subject the biological sample to centrifugation to yield a solution comprising the biological sample and a pellet, or to yield a pellet comprising the biological sample and a supernatant.

In some embodiments, after incubating the mixture of the biological sample and the lysis buffer, the one or more computer processors may be individually or collectively programmed to subject the mixture to centrifugation to yield a supernatant comprising the biological sample. Then, the supernatant may serve as the mixture for subsequent steps. For example, the supernatant may be added to a reaction vessel comprising reagents necessary for conducting nucleic acid amplification.

In a system of the present disclosure, the one or more computer processors may be individually or collectively programmed to cultivate the biological sample for microbial proliferation. In some embodiments, prior to mixing the biological sample with a lysis buffer, the one or more computer processors may be individually or collectively programmed to subject the biological sample to enrichment culturing conditions for a culturing time period. The enrichment culturing conditions may comprise culturing the biological sample in a suitable culture medium (e.g., tryptic soy broth, modified tryptic soy broth, tryptone, nutrient broth, L-broth, gram negative broth, peptone, tryptic soy broth with yeast, or Salmonella medium) at a suitable temperature (e.g., from 23° C. to 40° C., such as 25° C., 30° C., 35° C., or 37° C.) with or without shaking. In some embodiment, the culture medium is a Salmonella medium which favors proliferation of Salmonella as compared to other bacteria. Exemplary Salmonella medium includes Bismuth Sulfite agar (BS), xylose lysine deoxycholate agar (XLD), selenite brilliant green sulfa medium (SBG), but is not limited thereto. The culturing time period may be from about 0.5 hour to 5 hours, e.g., about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, or about 10 hours. In some embodiments, the culturing time period is no more than about 7 hours, e.g., no more than about 6.5 hours, no more than about 6 hours, no more than about 5.5 hours, no more than about 5 hour, no more than about 4.5 hour, no more than about 4 hours, no more than about 3.5 hours, no more than about 3 hours, no more than about 2.5 hours, no more than about 2 hours, no more than about 1.5 hours, no more than about 1 hour, or no more than about 0.5 hour.

In some embodiments, prior to and/or after subjecting the biological sample to enrichment culturing conditions for a culturing time period, the one or more computer processors are individually or collectively programmed to subject the biological sample to centrifugation to yield a solution comprising the biological sample and a pellet, or to yield a pellet comprising the biological sample and a supernatant.

In some embodiments, after subjecting the biological sample to enrichment culturing conditions for a culturing time period, the one or more computer processors are individually or collectively programmed to mix the biological sample with the lysis buffer without selective enrichment, plating on differential medium, and/or presumptive biomedical identification.

In some embodiments, after the biological sample has been subjected to enrichment culturing conditions for a culturing time period, the one or more computer processors may be individually or collectively programmed to add a lysis buffer to the mixture. The lysis buffer may be alkaline. For example, the lysis buffer comprises NaOH. The lysis buffer may have a pH from about 7 to 14, such as from about 8 to 13, from about 9 to 12, from about 10 to 11. For example, the lysis buffer may have a pH of about 7, 7.5, 8, 8.5, 9, 9.5, 10, 10.5, 11, 11.5, 12, 12.5, 13, 13.5, or 14.

In some embodiments, the one or more computer processors may be individually or collectively programmed to add the mixture of the biological sample with the lysis buffer to the reaction vessel without being subject to DNA or RNA extraction. In some cases, the one or more computer processors may be individually or collectively programmed to add the mixture of the biological sample with the lysis buffer to the reaction vessel without being purified. In some cases, the one or more computer processors may be individually or collectively programmed to add the mixture of the biological sample with the lysis buffer to the reaction vessel without being subject to DNA or RNA concentration. The reaction vessel may be one that comprises reagents necessary for conducting nucleic acid amplification.

In some embodiments, the one or more computer processors may be individually or collectively programmed to provide a biological sample obtained from a subject or the mixture, supernatant, or homogenized preparation derived from the biological sample as described elsewhere herein with reagents necessary for nucleic acid amplification in a reaction vessel to obtain a reaction mixture. The reaction vessel may be any reaction vessel as described herein.

The system of the present disclosure may further comprise a recognition module for recognizing information included in a kit for use in conjunction with the system in accordance with the present disclosure. For example, the kit may be tagged with a unique identifier. The unique identifier may be a barcode. The barcode may be a one-dimensional or two-dimensional barcode. The barcode may allow extraction of information from the kit by scanning. The unique identifier may involve contactless technique. The contactless technique allows extraction of information on the kit by placing the kit in proximity to a contactless detector. The contactless detector may use RFID (radio frequency identification) technique to extract information from the kit. In some embodiments, the unique identifier may be an RFID tag.

In some embodiment, the recognition module may comprise a barcode scanning module for scanning the barcode on the kit to extract the information. In some embodiments, the recognition module may comprise an RFID module (e.g., a contactless detector) for extracting information from the kit using RFID technique. In some embodiments, the recognition module comprises both the barcode scanning module and the RFID module.

The recognition module may be operatively connected to or in communication to one or more other modules as described herein, thereby transmitting the extracted information to the one or more other modules. The information may be relevant parameters for conducting a primer extension reaction. For example, the recognition module may communicate with the amplification module, thereby transmitting the relevant parameters to the amplification module for conducting the primer extension reaction. Once receiving the parameters, the amplification module may automatically conduct the primer extension reaction in accordance with the parameters. The parameters may be, e.g., the number of series of the primer extension reaction, the number of cycles in each series, denaturing condition, elongation condition, one or more primer sets, reporter agent(s), oligonucleotide probe(s), and the like, but not limited thereto.

In a system of the present disclosure, the one or more computer processors may be individually or collectively programmed to detect the amount and/or presence of the amplified product(s). In some cases, the one or more computer processors may be individually or collectively programmed to output information indicative of an amount and/or presence of said amplified product(s) to a recipient.

The system of the present disclosure may further comprise a detection module for detecting the amount and/or presence of the amplified product(s). The detection module may use any method of detection as described herein to detect the amount and/or presence of the amplified product(s). For example, the detection module may detect the detectable signal produced during amplification. For example, the detection module may detect the detectable signal produced by the reporter agent as described herein. The detectable signal may be indicative of whether the amplified product(s) is present. For example, the present or absence of the detectable signal may be indicative of the presence of the amplified product(s). The intensity of the detectable signal may be proportional to the amount of amplified product(s). In some cases, where amplified product(s) is generated of a different type of nucleic acid than the target nucleic acid initially amplified, the intensity of the detectable signal may be proportional to the amount of target nucleic acid initially amplified.

In some embodiments, the detection module may be an optical detection module. The optical detection module may detect the optical signal produced during the amplification. The optical signal may be an optical signal produced by any optically active dye as described herein. For example, the optical signal may be an optical signal accompanying the production of the amplified product(s). For example, the optical signal may be an optical signal produced by an oligonucleotide probe upon breakdown. For example, the optical signal may be an optical signal produced when a molecular beacon hybridizes with the amplified product(s).

In some embodiments, the optical signal may be a fluorescent signal, while the detection module may be a fluorescence detection module. For example, the fluorescence detection module may detect the fluorescent signal produced by any fluorescent dye as described herein. For example, the fluorescence detection module may detect the fluorescent signal produced by one or more fluorescent dyes selected from the group consisting of FAM, TET, ROX, JOE, HEX, TAMRA, VIC, NET, PET, Texas Red, and the like.

In some embodiments, the detection module may detect a plurality of detectable signals in parallel. For example, the plurality of detectable signals is produced by a plurality of reporter agents, while the plurality of detectable signals produced by the plurality of reporter agents is distinct among one another. Each of the plurality of detectable signals may be indicative of the presence or absence of the corresponding amplified product. The intensity of each of the plurality of detectable signals may be proportional to the amount of the corresponding amplified product. For example, the detection module may detect FAM fluorescence and ROX fluorescence in parallel. In such a circumstance, the detectable signal corresponding to one amplified product may be FAM fluorescence, while the detectable signal corresponding to another amplified product may be ROX fluorescence, thereby enabling the detection of distinct detectable signals in parallel. Detection of distinct detectable signals in parallel may allow comparison of the presence and/or amount among different amplified products. Alternatively or additionally, detection of distinct detectable signals in parallel may allow determination of the amount of the amplified product(s) relative to an internal reference.

In some cases, before conducting the primer extension reactions, the one or more computer processors may be individually or collectively programmed to subject the target nucleic acid molecule to one or more denaturing conditions. The one or more denaturing conditions may be selected from a denaturing temperature profile and a denaturing agent.

In some cases, before conducting the primer extension reactions, the one or more computer processors may be individually or collectively programmed to preheat the biological sample at a preheating temperature between about 90° C. to 100° C. for a preheating duration no longer than about 10 minutes. In some embodiments, the preheating duration is no longer than 1 minute.

In any of the various aspects, the one or more computer processors may be individually or collectively programmed to conduct multiple cycles of a primer extension reaction. Any suitable number of cycles may be conducted. For example, the number of cycles conducted may be less than about 100, 90, 80, 70, 60, 50, 40, 30, 20, 10, or 5 cycles.

In some embodiments, the one or more computer processors may be individually or collectively programmed to subject a reaction mixture to a plurality of series of primer extension reactions. An individual series of the plurality may comprise multiple cycles of a particular primer extension reaction, characterized, for example, by particular denaturation and elongation conditions as described elsewhere herein. Generally, each individual series differs from at least one other individual series in the plurality with respect to, for example, a denaturation condition and/or elongation condition. An individual series may differ from another individual series in a plurality of series, for example, with respect to any one, two, three, four, or all five of ramping rate, denaturing temperature, denaturing duration, elongation temperature, and elongation duration. Moreover, a plurality of series may comprise any number of individual series such as, for example, at least about or about 2, 3, 4, 5, 6, 7, 8, 9, 10, or more individual series.

In some embodiments, the one or more computer processors may be individually or collectively programmed to subject the target nucleic acid molecule to one or more denaturing conditions between a first series and a second series of the plurality of series of primer extension reactions. The individual series may differ with respect to at least any one (e.g., at least any two) of ramping rate between denaturing temperature and elongation temperature, denaturing temperature, denaturing duration, elongation temperature and elongation duration. Moreover, the plurality of series of primer extension reactions may comprise a first series and a second series. For example, the first series may comprise more than ten cycles, with each cycle of the first series comprising (i) incubating the reaction mixture at about 92° C.-95° C. for no more than 30 seconds, followed by (ii) incubating the reaction mixture at about 35° C.-65° C. for no more than 1 minute. The second series may comprise more than ten cycles, with each cycle of the second series comprising (i) incubating the reaction mixture at about 92° C.-95° C. for no more than 30 seconds, followed by (ii) incubating the reaction mixture at about 40° C.-60° C. for no more than 1 minute.

For example, the one or more computer processors may be individually or collectively programmed to conduct a plurality of series of primer extension reactions comprising a first series and a second series. The first series, for example, may comprise more than ten cycles of a primer extension reaction, where each cycle of the first series comprises (i) incubating a reaction mixture at about 92° C. to about 95° C. for no more than 30 seconds followed by (ii) incubating the reaction mixture at about 35° C. to about 65° C. for no more than about one minute. The second series, for example, may comprise more than ten cycles of a primer extension reaction, where each cycle of the second series comprises (i) incubating the reaction mixture at about 92° C. to about 95° C. for no more than 30 seconds followed by (ii) incubating the reaction mixture at about 40° C. to about 60° C. for no more than about 1 minute. In this particular example, the first and second series differ in their elongation temperature condition. The example, however, is not meant to be limiting as any combination of different elongation and denaturing conditions may be used.

In some embodiments, the one or more computer processors may be individually or collectively programmed to subject a target nucleic acid to a denaturing condition prior to initiation of a primer extension reaction. In the case of a plurality of series of primer extension reactions, the one or more computer processors may be individually or collectively programmed to subject the target nucleic acid to a denaturing condition prior to executing the plurality of series or to a denaturing condition between series of the plurality. For example, the one or more computer processors may be individually or collectively programmed to subject the target nucleic acid to a denaturing condition between a first series and a second series of a plurality of series. Non-limiting examples of such denaturing conditions include a denaturing temperature profile (e.g., one or more denaturing temperatures) and a denaturing agent.

In some embodiments, the one or more computer processors may be individually or collectively programmed to preheat a biological sample prior to conducting a primer extension reaction. The temperature (e.g., a preheating temperature) at which and duration (e.g., a preheating duration) for which a biological sample is preheated may vary depending upon, for example, the particular biological sample being analyzed. In some examples, a biological sample may be preheated for no more than about 60 minutes, 50 minutes, 40 minutes, 30 minutes, 25 minutes, 20 minutes, 15 minutes, 10 minutes, 9 minutes, 8 minutes, 7 minutes, 6 minutes, 5 minutes, 4 minutes, 3 minutes, 2 minutes, 1 minute, 45 seconds, 30 seconds, 20 seconds, 15 seconds, 10 seconds, or 5 seconds. In some examples, a biological sample may be preheated at a temperature from about 80° C. to about 110° C. In some examples, a biological sample may be preheated at a temperature from about 90° C. to about 100° C. In some examples, a biological sample may be preheated at a temperature from about 90° C. to about 97° C. In some examples, a biological sample may be preheated at a temperature from about 92° C. to about 95° C. In still other examples, a biological sample may be preheated at a temperature of about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

In some embodiments, the system of the present disclosure includes an electronic display screen that has a user interface that displays a graphical element that is accessible by a user to execute an amplification protocol to amplify the target nucleic acid in the biological sample. The system can also include a computer processor (including any suitable device having a computer processor as described elsewhere herein) coupled to the electronic display screen and programmed to execute the amplification protocol upon selection of the graphical element by the user. The amplification protocol can comprise subjecting a reaction mixture comprising the biological sample and reagents necessary for conducting nucleic acid amplification to a plurality of series of primer extension reactions to generate amplified product. The amplified product can be indicative of the presence of the target nucleic acid in the biological sample. Moreover, each series of primer extension reactions can comprise two or more cycles of incubating the reaction mixture under a denaturing condition that is characterized by a denaturing temperature and a denaturing duration, followed by incubating the reaction mixture under an elongation condition that is characterized by an elongation temperature and an elongation duration. An individual series can differ from at least one other individual series of the plurality with respect to the denaturing condition and/or the elongation condition.

In some embodiments, the target nucleic acid may be associated with a disease. The disease may be, for example, associated with an RNA virus or a DNA virus. Examples of viruses are provided elsewhere herein (e.g., Coxsachie virus A16). In some embodiments, the disease may be associated with a pathogenic bacterium (such as Mycobacterium tuberculosis or Salmonella) or a pathogenic protozoan (e.g., Plasmodium as in Malaria), as described elsewhere in the specification. In some embodiments, the amplification protocol can be directed to assaying for the presence of said disease based on a presence of the amplified product.

In some cases, a user interface can be a graphical user interface. Moreover, a user interface can include one or more graphical elements. Graphical elements can include image and/or textual information, such as pictures, icons and text. The graphical elements can have various sizes and orientations on the user interface. Furthermore, an electronic display screen may be any suitable electronic display including examples described elsewhere herein. Non-limiting examples of electronic display screens include a monitor, a mobile device screen, a laptop computer screen, a television, a portable video game system screen and a calculator screen. In some embodiments, an electronic display screen may include a touch screen (e.g., a capacitive or resistive touch screen) such that graphical elements displayed on a user interface of the electronic display screen can be selected via user touch with the electronic display screen.

In some embodiments, the amplification protocol may further include selecting a primer set for the target nucleic acid. In such cases, the primer set may be a primer set specifically designed to amplify one or more sequences of the target nucleic acid molecule. In some embodiments, the amplification protocol may further include selecting a reporter agent (e.g., an oligonucleotide probe comprising an optically-active species or other type of reporter agent described elsewhere herein) that is specific for one or more sequences of the target nucleic acid molecule. Moreover, in some embodiments, the reagents may comprise any suitable reagents necessary for nucleic acid amplification as described elsewhere herein, such as, for example, a deoxyribonucleic acid (DNA) polymerase, a primer set for the target nucleic acid, and, in some cases, a reverse transcriptase.

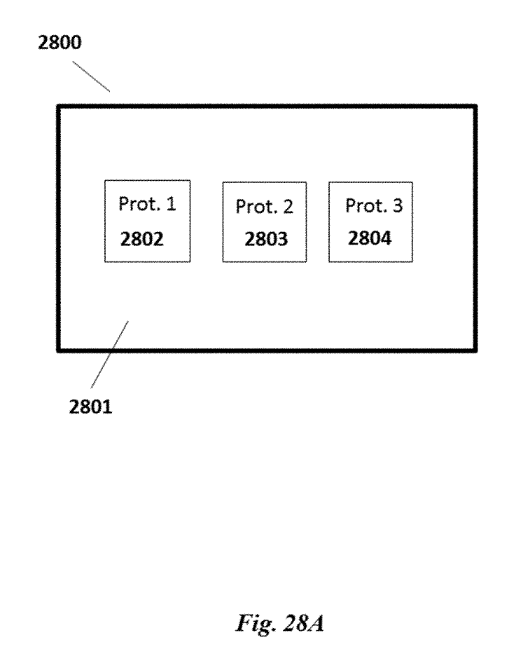

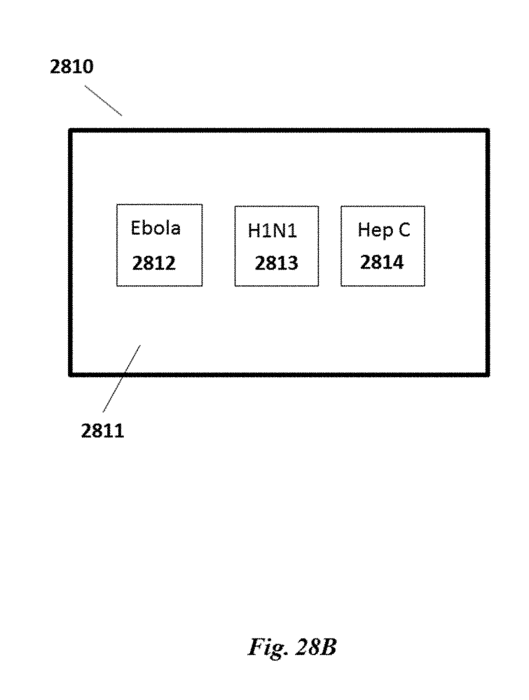

In some embodiments, the user interface can display a plurality of graphical elements. Each of the graphical elements can be associated with a given amplification protocol among a plurality of amplification protocols. Each of the plurality of amplification protocols may include a different combination of series of primer extension reaction. In some cases, though, a user interface may display a plurality of graphical elements associated with the same amplification protocol. An example of a user interface having a plurality of graphical elements each associated with a given amplification protocol is shown in FIG. 28A. As shown in FIG. 28A, an example electronic display screen 2800 associated with a computer processor includes a user interface 2801. The user interface 2801 includes a display of graphical elements 2802, 2803 and 2804. Each of the graphical elements can be associated with a particular amplification protocol (e.g., “Prot. 1” for graphical element 2802, “Prot. 2” for graphical element 2803 and “Prot. 4” for graphical element 2804). Upon user selection (e.g., user touch when the electronic display screen 2800 includes a touch-screen having the user interface) of particular graphical element, the particular amplification protocol associated with the graphical element can be executed by an associated computer processor. For example, when a user selects graphical element 2803, amplification “Prot. 2” is executed by the associated computer processor. Where only three graphical elements are shown in the example user interface 2801 of FIG. 28A, a user interface may have any suitable number of graphical elements. Moreover, where each graphical element shown in the user interface 2801 of FIG. 28A is associated with only one amplification protocol, each graphical element of a user interface can be associated with one or more amplification protocols (e.g., a series of amplification protocols) such that an associated computer processor executes a series of amplification protocols upon user interaction with the graphical element.

In some embodiments, each of the graphical elements and/or may be associated with a disease, and a given amplification protocol among the plurality of amplification protocols may be directed to assaying a presence of the disease in the subject. Thus, in such cases, a user can select a graphical element in order to run an amplification protocol (or series of amplification protocols) to assay for a particular disease. In some embodiments, the disease may be associated with a virus such as, for example, any RNA virus or DNA virus including examples of such viruses described elsewhere herein. Non-limiting examples of viruses include human immunodeficiency virus I (HIV I), human immunodeficiency virus II (HIV II), an orthomyxovirus, Ebola virus, Dengue virus, influenza viruses (e.g., H1N1 virus, H3N2 virus, H7N9 virus or H5N1 virus), hepevirus, hepatitis A virus, hepatitis B virus, hepatitis C virus (e.g., armored RNA-hepatitis C virus (RNA-HCV)), hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus (e.g., adenovirus type 55 (ADV55), adenovirus type 7 (ADV7)) Varicella virus, enterovirus, and norovirus. In some embodiments, the disease may be associated with a pathogenic bacterium (e.g., Mycobacterium tuberculosis) or a pathogenic protozoan (e.g., Plasmodium as in Malaria), including examples of such pathogens described elsewhere herein.

An example of a user interface having a plurality of graphical elements each associated with a given amplification protocol is shown in FIG. 28B. As shown in FIG. 28B, an example electronic display screen 2810 associated with a computer processor includes a user interface 2811. The user interface 2811 includes a display of graphical elements 2812, 2813 and 2814. Each of the graphical elements can be associated with a particular disease (e.g., “Ebola” for graphical element 2812, “H1N1” for graphical element 2813 and “Hep C” (Hepatitis C) for graphical element 2814) that is, in turn, associated with one or more amplification protocols directed toward the particular disease. Upon user selection (e.g., user touch when the electronic display screen 2810 includes a touch-screen having the user interface) with a particular graphical element, the particular amplification protocol(s) associated with the disease associated with the graphical element can be executed by an associated computer processor. For example, when a user interacts with graphical element 2812, one or more amplification protocols associated with assaying for Ebola virus can be executed by the associated computer processor. Where only three graphical elements are shown in the example user interface 2811 of FIG. 28B, a user interface may have any suitable number of graphical elements each corresponding to a various disease. Moreover, where each graphical element shown in the user interface 2811 of FIG. 28B is associated with only one disease, each graphical element of a user interface can be associated with one or more diseases such that an associated computer processor executes a series of amplification protocols (e.g., each individual amplification protocol directed to a particular disease) upon user selection of the graphical element. For example, a graphical element may correspond to Ebola virus and H1N1 virus such that selection of the graphical element results in an associated computer processor executing amplification protocols for both Ebola virus and H1N1 virus.

In various aspects, the system may comprise an input module that receives a user request to amplify a target nucleic acid (e.g., target RNA, target DNA) present in a biological sample. The biological sample may be obtained direct from a subject. Any suitable module capable of accepting such a user request may be used. The input module may comprise, for example, a device that comprises one or more processors. Non-limiting examples of devices that comprise processors (e.g., computer processors) include a desktop computer, a laptop computer, a tablet computer (e.g., Apple® iPad, Samsung® Galaxy Tab), a cell phone, a smart phone (e.g., Apple® iPhone, Android® enabled phone), a personal digital assistant (PDA), a video-game console, a television, a music playback device (e.g., Apple® iPod), a video playback device, a pager, and a calculator. Processors may be associated with one or more controllers, calculation units, and/or other units of a computer system, or implanted in firmware as desired. If implemented in software, the routines (or programs) may be stored in any computer readable memory such as in RAM, ROM, flash memory, a magnetic disk, a laser disk, or other storage medium. Likewise, this software may be delivered to a device via a delivery method including, for example, over a communication channel such as a telephone line, the internet, a local intranet, a wireless connection, etc., or via a transportable medium, such as a computer readable disk, flash drive, etc. The various steps may be implemented as various blocks, operations, tools, modules or techniques which, in turn, may be implemented in hardware, firmware, software, or any combination thereof. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

In some embodiments, the input module is configured to receive a user request to perform amplification of the target nucleic acid. The input module may receive the user request directly (e.g., by way of an input device such as a keyboard, mouse, or touch screen operated by the user) or indirectly (e.g., through a wired or wireless connection, including over the internet). Via output electronics, the input module may provide the user's request to the amplification module. In some embodiments, an input module may include a user interface (UI), such as a graphical user interface (GUI), that is configured to enable a user provide a request to amplify the target nucleic acid. A GUI can include textual, graphical and/or audio components. A GUI can be provided on an electronic display, including the display of a device comprising a computer processor. Such a display may include a resistive or capacitive touch screen.

Non-limiting examples of users include the subject from which the biological sample was obtained, medical personnel, clinicians (e.g., doctors, nurses, laboratory technicians), laboratory personnel (e.g., hospital laboratory technicians, research scientists, pharmaceutical scientists), a clinical monitor for a clinical trial, or others in the health care industry.

In various aspects, the system comprises an amplification module for performing nucleic acid amplification reaction on target nucleic acid or a portion thereof, in response to a user request received by the input module. The amplification module may be capable of executing any of the methods described herein and may include any of a fluid handling device, one or more thermocyclers, a device or module for receiving one or more reaction vessels (e.g., wells of a thermal block of a thermocycler), a detector (e.g., optical detector, spectroscopic detector, electrochemical detector) capable of detecting amplified product, and a device or module for outputting information (e.g., raw data, processed data, or any other type of information described herein) regarding the presence and/or amount of amplified product (e.g., amplified DNA product) to a recipient. In some cases, the amplification module may comprise a device with a computer processor as described elsewhere herein and may also be capable of analyzing raw data from detection, with the aid of appropriate software. Moreover, in some embodiments, the amplification module may comprise input electronics necessary to receive instructions from the input module and may comprise output electronics necessary to communicate with the output module.

In some embodiments, one or more steps of providing materials to a reaction vessel, amplification of nucleic acids, detection of amplified product, and outputting information may be automated by the amplification module. In some embodiments, automation may comprise the use of one or more fluid handlers and associated software. Several commercially available fluid handling systems can be utilized to run the automation of such processes. Non-limiting examples of such fluid handlers include fluid handlers from Perkin-Elmer, Caliper Life Sciences, Tecan, Eppendorf, Apricot Design, and Velocity 11.

In some embodiments, an amplification module may include a real-time detection instrument. Non-limiting examples of such instruments include a real-time PCR thermocycler, ABI PRISM® 7000 Sequence Detection System, ABI PRISM® 7700 Sequence Detection System, Applied Biosystems 7300 Real-Time PCR System, Applied Biosystems 7500 Real-Time PCR System, Applied Biosystems 7900 HT Fast Real-Time PCR System (all from Applied Biosystems); LightCycler™ System (Roche Diagnostics GmbH); Mx3000P™ Real-Time PCR System, Mx3005P™ Real-Time PCR System, and Mx4000® Multiplex Quantitative PCR System (Stratagene, La Jolla, Calif.); and Smart Cycler System (Cepheid, distributed by Fisher Scientific). In some embodiments, an amplification module may comprise another automated instrument such as, for example, a COBAS® AmpliPrep/COBAS® TaqMan® system (Roche Molecular Systems), a TIGRIS DTS system (Hologic Gen-Probe, San Diego, Calif.), a PANTHER system (Hologic Gen-Probe, San Diego, Calif.), a BD MAX™ system (Becton Dickinson), a GeneXpert System (Cepheid), a Filmarray® (BioFire Diagnostics) system, an iCubate system, an IDBox system (Luminex), an EncompassMDx™ (Rheonix) system, a Liat™ Aanlyzer (IQuum) system, a Biocartis' Molecular Diagnostic Platform system, an Enigma® ML system (Enigma Diagnostics), a T2Dx® system (T2 Biosystems), a Verigene® system (NanoSphere), a Great Basin's Diagnostic System, a Unyvero™ System (Curetis), a PanNAT system (Micronics), or a Spartan™ RX system (Spartan Bioscience).

In various aspects, the system may comprise an output module operatively connected to the amplification module. In some embodiments the output module may comprise a device with a processor as described above for the input module. The output module may include input devices as described herein and/or may comprise input electronics for communication with the amplification module. In some embodiments, the output module may be an electronic display, in some cases the electronic display comprising a UI. In some embodiments, the output module is a communication interface operatively coupled to a computer network such as, for example, the internet. In some embodiments, the output module may transmit information to a recipient at a local or remote location using any suitable communication medium, including a computer network, a wireless network, a local intranet, or the internet. In some embodiments, the output module is capable of analyzing data received from the amplification module. In some cases, the output module includes a report generator capable of generating a report and transmitting the report to a recipient, wherein the report contains any information regarding the amount and/or presence of amplified product as described elsewhere herein. In some embodiments, the output module may transmit information automatically in response to information received from the amplification module, such as in the form of raw data or data analysis performed by software included in the amplification module. Alternatively, the output module may transmit information after receiving instructions from a user. Information transmitted by the output module may be viewed electronically or printed from a printer.

One or more of the input module, biological sample treatment module, recognition module, detection module, amplification module, and output module may be contained within the same device or may comprise one or more of the same components. For example, an amplification module may also comprise an input module, a biological sample treatment module, a recognition module, a detection module, an output module, or two or more of them. In other examples, a device comprising a processor may be included in both the input module and the output module. A user may use the device to request that a target nucleic acid be amplified and may also be used to transmit information regarding amplified product to a recipient. In some cases, a device comprising a processor may be included in all six modules, such that the device comprising a processor may also be used to control, provide instructions to, and receive information back from instrumentation (e.g., a thermocycler, a detector, an incubator, a fluid handling device) included in the amplification module or any other module. Each of the six modules may further comprise any of the one or more computer processors as described herein, and/or may execute the function that the one or more computer processors are programmed to execute.

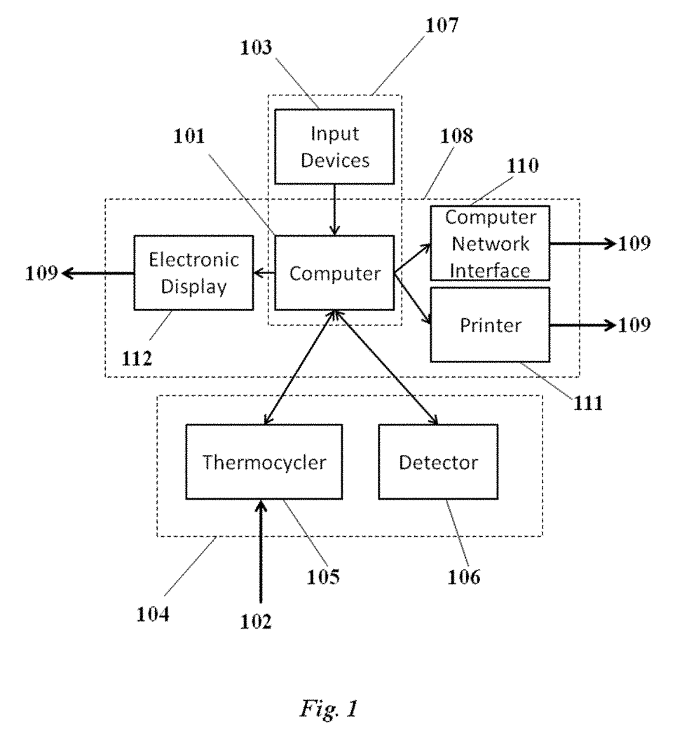

An example system for amplifying a target nucleic acid according to methods described herein is depicted in FIG. 1. The system comprises a computer 101 that may serve as part of both the input and output modules. A user enters a reaction vessel 102 comprising a reaction mixture ready for nucleic acid amplification into the amplification module 104. The amplification module comprises a thermocycler 105 and a detector 106. The input module 107 comprises computer 101 and associated input devices 103 (e.g., keyboard, mouse, etc.) that can receive the user's request to amplify a target nucleic acid in the reaction mixture. The input module 107 communicates the user's request to the amplification module 104 and nucleic acid amplification commences in the thermocycler 105. As amplification proceeds, the detector 106 of the amplification module detects amplified product. Information (e.g., raw data obtained by the detector) regarding the amplified product is transmitted from the detector 106 back to the computer 101, which also serves as a component of the output module 108. The computer 101 receives the information from the amplification module 104, performs any additional manipulations to the information, and then generates a report containing the processed information. Once the report is generated, the computer 101 then transmits the report to its end recipient 109 over a computer network (e.g., an intranet, the internet) via computer network interface 110, in hard copy format via printer 111, or via the electronic display 112 operatively linked to computer 101.

In another aspect, the present disclosure provides a kit for the purpose of the present disclosure, for example, for conducting the methods of the present disclosure. The kit may comprise one or more elements in any combination in accordance with any of the various aspects of the present disclosure.

In some embodiments, the present disclosure provides a kit for detecting a target nucleic acid molecule in a biological sample. The kit comprises:

(a) one or more primer sets for said target nucleic acid molecule;

(b) enzyme(s) necessary for conducting nucleic acid amplification, such as a deoxyribonucleic acid (DNA) polymerase and optionally a reverse transcriptase;

(c) buffer(s) necessary for conducting the nucleic acid amplification;

(d) nucleotides and analogs thereof capable of being incorporated by the DNA polymerase in an amplification reaction, such as dNTPs; and

(e) optionally, reporter agent(s), such as reporter agent(s) that yields a detectable signal indicative of a presence of the amplified product(s) of the nucleic acid amplification,

The kit may further comprise a negative control, a positive control, and/or an internal reference or other reference for quantification.

The one or more primer sets, the enzyme(s) necessary for conducting nucleic acid amplification, the buffer(s) necessary for conducting the nucleic acid amplification, the reporter agent, the nucleotides and analogs thereof, the negative control, the positive control, the internal reference or other reference for quantification may be any one of those as described elsewhere herein. The kit may further comprise other reagent(s) necessary for conducting the nucleic acid amplification. The reagent(s) necessary for conducting the nucleic acid amplification may be any of those as described elsewhere herein. The kit may further comprise any reagent, buffer, or other material for treating, pre-treating, cultivating, enriching, or identifying any sample as described elsewhere herein, including any of those reagents, buffers, or other materials as described herein. For example, the kit may comprise the suspension buffer, lysis buffer, and the like as described herein.

The reagents or other materials in the kit may be provided in any suitable container, including but not limited to test tubes, vials, flasks, bottles, ampules, syringes, or the like. The reagents or other materials may be provided in a form that may be ready for use in the methods of the present disclosure, or in a form that needs to be combined with other reagents in the kit or reagents provided by the user (for example, dilution of concentrated compositions or reconstitution of lyophilized compositions) prior to use. The buffer that may be provided in the kit includes, but is not limited to saline, NaOH solution, sodium carbonate buffer, sodium bicarbonate buffer, borate buffer, Tris buffer, MOPS buffer, HEPES buffer, phosphate buffer, any other buffer as described elsewhere herein, or combination thereof. The kit may comprise control samples, for example, purified DNA used for microbes of known species and quantity/concentration, and/or used as a positive control, internal reference, or other reference for quantification, as well as a negative control that is known to be insusceptible to nucleic acid amplification. In some embodiments, the kit further comprises instructions for using the kit in accordance with one or more methods of the present disclosure.

In some embodiments, the present disclosure provides a kit for detecting Salmonella in a biological sample. The biological sample may be a stool sample. The kit may comprises:

(a) one or more primer sets, each capable of specifically binding to a target nucleic acid sequence from a Salmonella genome or a Salmonella transcriptome, or a variant thereof, for amplifying the target nucleic acid sequence in an amplification reaction to obtain amplification product(s),

(b) a deoxyribonucleic acid (DNA) polymerase and optionally a reverse transcriptase,

(c) buffer(s) for a nucleic acid amplification,

(d) nucleotides and analogs thereof capable of being incorporated by the DNA polymerase in an amplification reaction,

(e) optionally, reporter agent(s) that yields a detectable signal indicative of a presence of the amplified product(s), and

(f) optionally, instructions for using the one or more primers sets, DNA polymerase, and nucleotides and analogs thereof to perform nucleic acid amplification to detect Salmonella in the biological sample.

In some embodiments, the nucleotides and analogs thereof capable of being incorporated by the DNA polymerase in an amplification reaction are dNTPs.

The one or more primer set may comprise a forward primer. The forward primer may be selected from the group consisting of SEQ ID NO: 1 (TGCTCGTTTACGACCTGAATTA), SEQ ID NO: 4 (TCGTTTACGACCTGAATTAC), and SEQ ID NO: 7 (CTCACCAGGAGATTACAACATGG). The one or more primer set may comprise a reverse primer. The reverse primer may be selected from the group consisting of SEQ ID NO: 2 (ACACCAATATCGCCAGTACG), SEQ ID NO: 5 (TAGAACGACCCCATAAACA), and SEQ ID NO: 8 (AGCTCAGACCAAAAGTGACCATC).

The one or more primer set may include a primer set consisting of a forward primer as depicted in SEQ ID NO: 1 (TGCTCGTTTACGACCTGAATTA) and a reverse primer depicted in SEQ ID NO: 2 (ACACCAATATCGCCAGTACG). Alternatively or additionally, the one or more primer set may include a primer set consisting of a forward primer as depicted in SEQ ID NO: 4 (TCGTTTACGACCTGAATTAC) and a reverse primer depicted in SEQ ID NO: 5 (TAGAACGACCCCATAAACA). Alternatively or additionally, the one or more primer set may include a primer set consisting of a forward primer as depicted in SEQ ID NO: 7 (CTCACCAGGAGATTACAACATGG) and a reverse primer depicted in SEQ ID NO: 8 (AGCTCAGACCAAAAGTGACCATC).

In some embodiments, the reporter agent may be a sequence-specific oligonucleotide probe. The sequence-specific oligonucleotide probe may comprise nucleic acid sequences selected from the group consisting of SEQ ID NO: 3 (TCTGGTTGATTTCCTGATCGCACTGA), SEQ ID NO: 6 (CTGGTTGATTTCCTGATCGCACT), and SEQ ID NO: 9 (CACCGACGGCGAGACCGACTTT).

In some embodiments, the method for using the kit may include any method as described herein. For example, the kit may be used for detecting Salmonella in a biological sample comprising:

(a) mixing the stool sample with a lysis buffer to obtain a mixture;

(b) incubating the mixture at an incubation temperature greater than 15° C. for an incubation time period of no more than about 15 minutes;

(c) adding the mixture from (b) to a reaction vessel comprising reagents necessary for conducting nucleic acid amplification, the reagents comprising (i) a deoxyribonucleic acid (DNA) polymerase and optionally a reverse transcriptase, and (ii) one or more primer sets, each capable of specifically binding to a target nucleic acid sequence from a Salmonella genome or a Salmonella transcriptome, or a variant thereof, to obtain a reaction mixture; and

(d) subjecting the reaction mixture in the reaction vessel to a plurality of series of primer extension reactions to generate amplified product(s) that is indicative of a presence of the target nucleic acid(s) in the sample, each series comprising two or more cycles of (i) incubating the reaction mixture under a denaturing condition characterized by a denaturing temperature and a denaturing duration, followed by (ii) incubating the reaction mixture under an elongation condition characterized by an elongation temperature and an elongation duration, wherein an individual series differs from at least one other individual series of the plurality with respect to the denaturing condition and/or the elongation condition. The biological sample may be a stool sample.

In some embodiments, the kit may further be tagged with a unique identifier. The unique identifier may be a barcode. The barcode may be a one-dimensional or two-dimensional barcode. The barcode may allow extraction of information from the kit by scanning. The unique identifier may involve contactless technique. The contactless technique allows extraction of information on the kit by placing the kit in proximity to a contactless detector. The contactless detector may use RFID (radio frequency identification) technique to extract information from the kit. In some embodiments, the unique identifier may be an RFID tag.

The information may be information about the components of the kit. The information may be information about the method for using the kit. The information may be information about any method, system, element, reagent, kit, computer assisted method and system, biological sample, and/or various materials as described herein.

In some embodiments, the system described herein may extract information from the kit by recognizing the unique identifier on the kit. For example, the system described herein may extract information (e.g., through the recognition module) from the kit by scanning the barcode on the kit or using RFID technique.

Once the information on the kit is extracted, the system described herein may automatically execute a method described herein based on the information. In some embodiments, the information relates to element(s) of the method described herein. In some embodiments, the information relates to sample pretreatment, cultivation or treatment. For example, the information may relate to incubation temperature, incubation time period, suspension buffer, lysis buffer, treatment duration, treatment conditions, but is not limited thereto. In some embodiments, the information relates to parameters for conducting a primer extension reaction described herein. For example, the information may relate to the number of series of the primer extension reaction, the number of cycles in each series, denaturing condition, elongation condition, one or more primer sets, reporter agent(s), oligonucleotide probe(s), or combinations thereof, but is not limited thereto. In some embodiments, the information allows the system described herein to automatically execute the method described herein without human intervention.

In another aspect, the present disclosure provides use of an agent for the manufacture of a kit for detecting a target nucleic acid molecule in a biological sample. In some embodiments, disclosure provides use of an agent for the manufacture of a kit for detecting Salmonella in a biological sample. The biological sample may be a stool sample. The agent may be any reagent or material as described herein. In some cases, the agent is a primer set as described herein. For example, the agent may be a primer set capable of specifically binding to a target nucleic acid sequence from a Salmonella genome or a Salmonella transcriptome, or a variant thereof.

In another aspect, the present disclosure provides a reaction mixture for the purpose of the present disclosure, for example, for conducting the methods of the present disclosure. The reaction mixture may comprise one or more elements in any combination in accordance with any of the various aspects of the present disclosure.

In some embodiments, the present disclosure provides a reaction mixture comprising:

(a) a biological material;

(b) one or more primer sets for target nucleic acid molecule(s);

(c) enzyme(s) necessary for conducting nucleic acid amplification, such as a deoxyribonucleic acid (DNA) polymerase and optionally a reverse transcriptase;

(d) optionally, buffer(s) necessary for conducting the nucleic acid amplification;

(e) nucleotides and analogs thereof capable of being incorporated by the DNA polymerase in an amplification reaction, such as dNTPs; and

(f) optionally, reporter agent(s), such as reporter agent(s) that yields a detectable signal indicative of a presence of the amplified product(s) of the nucleic acid amplification,

In some embodiments, the biological material may be a biological sample as described herein. The biological sample may be one that has been treated according to the method of the present disclosure. For example, the biological sample may be suspended in the suspension buffer. The biological sample may be mixed with the lysis buffer. For example, the biological sample may be mixed with the lysis buffer, followed by incubating at an incubation temperature for an incubation time period as described herein. The biological sample may be centrifuged to yield a supernatant prior to or after mixing with the lysis buffer. Here, the biological material may encompass the supernatant or lysate of the biological sample after mixing with the lysis buffer. In some embodiments, the biological sample may be a stool sample.

In some embodiments, the biological material may be a virus, bacterium, fungus or protozoan as described elsewhere herein. In some embodiments, the biological material may be a lysate of the virus, bacterium, fungus or protozoan. In some embodiments, the biological material may be a nucleic acid specific to or conservative among the individual species of the virus, bacterium, fungus or protozoan. The biological material may be any biological material that may serve as a template in an amplification reaction.

In some embodiments, the present disclosure provides a reaction mixture comprising:

(a) Salmonella, Salmonella lysate, or Salmonella nucleic acids,

(b) one or more primer sets, each capable of specifically binding to a target nucleic acid sequence from a Salmonella genome or a Salmonella transcriptome, or a variant thereof, for amplifying said target nucleic acid sequence in an amplification reaction to obtain amplification product(s),

(c) a deoxyribonucleic acid (DNA) polymerase and optionally a reverse transcriptase,

(d) nucleotides and analogs thereof capable of being incorporated by said DNA polymerase in an amplification reaction, and

(e) optionally, reporter agent(s) that yields a detectable signal indicative of a presence of said amplified product(s).

In some embodiments, the Salmonella may be Salmonella enterica. The Salmonella may be Salmonella bongori. The Salmonella may be any bacteria encompassed in the genus Salmonella, including but not limited to bacterial once considered as independent species of the genus Salmonella, such as Salmonella typhi, Salmonella typhimurium, Salmonella enteritidis, Salmonella choleraesuis, Salmonella paratyphi, Salmonella arizonae, and the like. The Salmonella may be one that has been treated h according to the method of the present disclosure. For example, the Salmonella may be a Salmonella lysate.

In some embodiments, the nucleotides and analogs thereof capable of being incorporated by the DNA polymerase in an amplification reaction are dNTPs.

The one or more primer set may comprise a forward primer. The forward primer may be selected from the group consisting of SEQ ID NO: 1 (TGCTCGTTTACGACCTGAATTA), SEQ ID NO: 4 (TCGTTTACGACCTGAATTAC), and SEQ ID NO: 7 (CTCACCAGGAGATTACAACATGG). The one or more primer set may comprise a reverse primer. The reverse primer may be selected from the group consisting of SEQ ID NO: 2 (ACACCAATATCGCCAGTACG), SEQ ID NO: 5 (TAGAACGACCCCATAAACA), and SEQ ID NO: 8 (AGCTCAGACCAAAAGTGACCATC).

The one or more primer set may include a primer set consisting of a forward primer as depicted in SEQ ID NO: 1 (TGCTCGTTTACGACCTGAATTA) and a reverse primer depicted in SEQ ID NO: 2 (ACACCAATATCGCCAGTACG). Alternatively or additionally, the one or more primer set may include a primer set consisting of a forward primer as depicted in SEQ ID NO: 4 (TCGTTTACGACCTGAATTAC) and a reverse primer depicted in SEQ ID NO: 5 (TAGAACGACCCCATAAACA). Alternatively or additionally, the one or more primer set may include a primer set consisting of a forward primer as depicted in SEQ ID NO: 7 (CTCACCAGGAGATTACAACATGG) and a reverse primer depicted in SEQ ID NO: 8 (AGCTCAGACCAAAAGTGACCATC).

In some embodiments, the reporter agent may be a sequence-specific oligonucleotide probe. The sequence-specific oligonucleotide probe may be selected from the group consisting of nucleic acid sequences as depicted in SEQ ID NO: 3 (TCTGGTTGATTTCCTGATCGCACTGA), SEQ ID NO: 6 (CTGGTTGATTTCCTGATCGCACT), and SEQ ID NO: 9 (CACCGACGGCGAGACCGACTTT).

The reaction mixture of the present disclosure may be contained in any suitable reaction site. The reaction site may be a container, such as a well of a multi-well plate, a plate, a tube, a chamber, a flow cell, a chamber or channel of a micro-fluidic device, or a chip. The reaction site may be a partition within a solution, such as a droplet (e.g. within an emersion mixture). In some embodiments, the reaction mixture is in a dehydrated form, such as a bead or film adhered to a surface of a container, a lyophilized powder, or a precipitate.

In an aspect, the present disclosure provides a computer readable medium comprising machine executable code that, upon execution by one or more computer processors, implements a method of the present disclosure. For example, the method maybe a method of detecting a target nucleic acid molecule in a biological sample. The method may comprise: (a) mixing said biological sample with a lysis buffer to obtain a mixture; (b) incubating said mixture at a temperature from about 15° C. to 70° C. for a period of time of no more than about 15 minutes; (c) adding said mixture from (b) to a reaction vessel comprising reagents necessary for conducting nucleic acid amplification, said reagents comprising (i) a deoxyribonucleic acid (DNA) polymerase, and (ii) a primer set for said target nucleic acid molecule, to obtain a reaction mixture; and (d) subjecting said reaction mixture in said reaction vessel to multiple cycles of a primer extension reaction to generate amplified product(s) that is indicative of a presence of said target nucleic acid molecule, each cycle comprising (i) incubating said reaction mixture at a denaturing temperature for a denaturing duration that is less than or equal to 60 seconds, followed by (ii) incubating said reaction mixture at an elongation temperature for an elongation duration that is less than or equal to 60 seconds, thereby amplifying said target nucleic acid molecule.

In another aspect, the present disclosure provides a computer readable medium comprising machine executable code that, upon execution by one or more computer processors, implements a method of detecting a target nucleic acid molecule in a biological sample. The method may comprise: (a) mixing said biological sample with a lysis buffer to obtain a mixture; (b) incubating said mixture at a temperature from about 15° C. to 70° C. at a period of time of no more than about 15 minutes; (c) adding said mixture from (b) to a reaction vessel comprising reagents necessary for conducting nucleic acid amplification, said reagents comprising (i) a deoxyribonucleic acid (DNA) polymerase and, in some cases, a reverse transcriptase, and (ii) a primer set for said target nucleic acid molecule, to obtain a reaction mixture; and (d) subjecting said reaction mixture in said reaction vessel to a plurality of series of primer extension reactions to generate amplified product(s) that is indicative of a presence of said target nucleic acid molecule in said sample, each series comprising two or more cycles of (i) incubating said reaction mixture under a denaturing condition characterized by a denaturing temperature and a denaturing duration, followed by (ii) incubating said reaction mixture under an elongation condition characterized by an elongation temperature and an elongation duration, wherein an individual series differs from at least one other individual series of said plurality with respect to said denaturing condition and/or said elongation condition.

Computer readable medium may take many forms, including but not limited to, a tangible (or non-transitory) storage medium, a carrier wave medium, or physical transmission medium. Non-volatile storage media include, for example, optical or magnetic disks, such as any of the storage devices in any computer(s) or the like, such as may be used to implement the calculation steps, processing steps, etc. Volatile storage media include dynamic memory, such as main memory of a computer. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

In another aspect, the present disclosure further provides primer sets and probes. The following primer sets and probes are used in the present disclosure.

FORWARD PRIMERS
(SEQ ID NO: 1)
TGCTCGTTTACGACCTGAATTA
(SEQ ID NO: 4)
TCGTTTACGACCTGAATTAC
(SEQ ID NO: 7)
CTCACCAGGAGATTACAACATGG
REVERSE PRIMERS
(SEQ ID NO: 2)
ACACCAATATCGCCAGTACG
(SEQ ID NO: 5)
TAGAACGACCCCATAAACA
(SEQ ID NO: 8)
AGCTCAGACCAAAAGTGACCATC
PROBES (SEQUENCE SPECIFIC PORTION ONLY)
(SEQ ID NO: 3)
TCTGGTTGATTTCCTGATCGCACTGA
(SEQ ID NO: 6)
CTGGTTGATTTCCTGATCGCACT
(SEQ ID NO: 9)
CACCGACGGCGAGACCGACTTT

### EXAMPLES

### Example 1: Amplification and Detection of Nucleic Acids in Viral Stock Samples and Biological Samples

Amplification and detection experiments were performed to compare results obtained from viral standard samples and biological samples. Biological samples comprising an RNA viral pathogen and standard samples of the viral pathogen were subject to amplification conditions, such that RNA of the pathogen was amplified. A set of experiments was conducted for each of the H3N2 and H1N1 (2007) influenza viruses. Each biological sample was obtained directly from a subject via an oropharyngeal swab. Each viral standard sample was obtained as a serial dilution of a stock solution comprising the virus. The concentrations of H3N2 and H1N1 (2007) were 10⁶ IU/mL. For H5N1 and H1N1 (2007), dilutions of ½, 1/20, 1/200, 1/2000, and 1/20000 were subject to amplification. In each experimental set, a negative control (e.g., a sample comprising no viral RNA) was also subject to amplification.

Five microliters of each sample were combined in a 25 μL reaction tube with reagents necessary to conduct reverse transcription of the viral RNA and reagents necessary to complete amplification of the complementary DNA obtained from reverse transcription (e.g., parallel nucleic acid amplification). The reagents necessary to conduct reverse transcription and DNA amplification were supplied as a commercially available pre-mixture (e.g., Qiagen One-Step RT-PCR or One-Step RT-qPCR kit) comprising reverse transcriptases (e.g., Sensiscript and Omniscript transcriptases), a DNA Polymerase (e.g., HotStarTaq DNA Polymerase), and dNTPs. Moreover, the reaction tubes also included a TaqMan probe comprising a FAM dye for detection of amplified DNA product. To generate amplified DNA product, each reaction mixture was incubated according to a protocol of denaturing and elongation conditions comprising 5 min at 95° C., followed by 20 min at 45° C., followed by 2 min at 95° C., and followed by 40 cycles of 5 seconds at 95° C. and 30 seconds at 55° C. in a real-time PCR thermocycler. Detection of amplified product occurred during incubations.

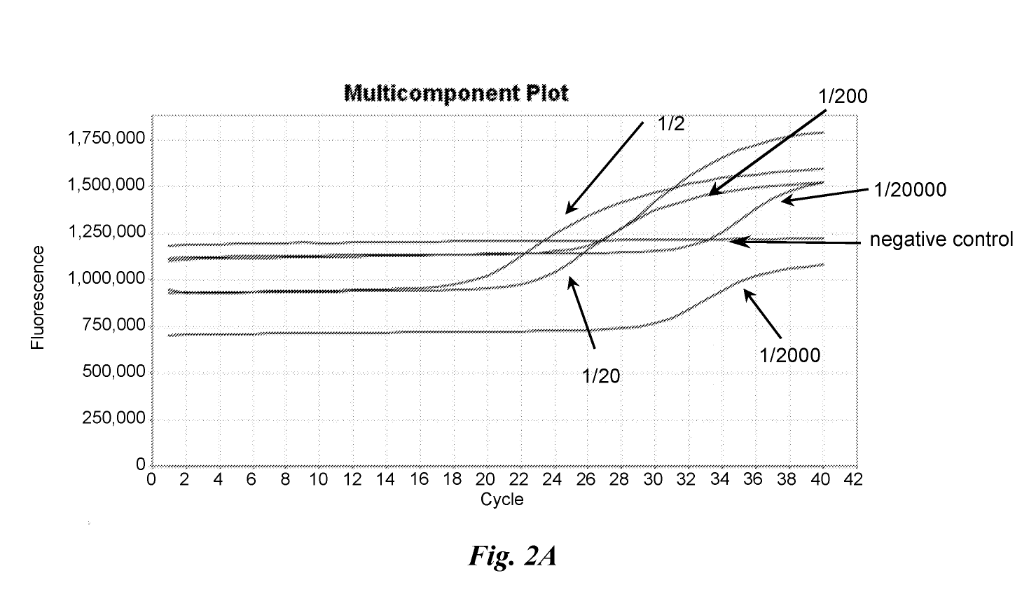

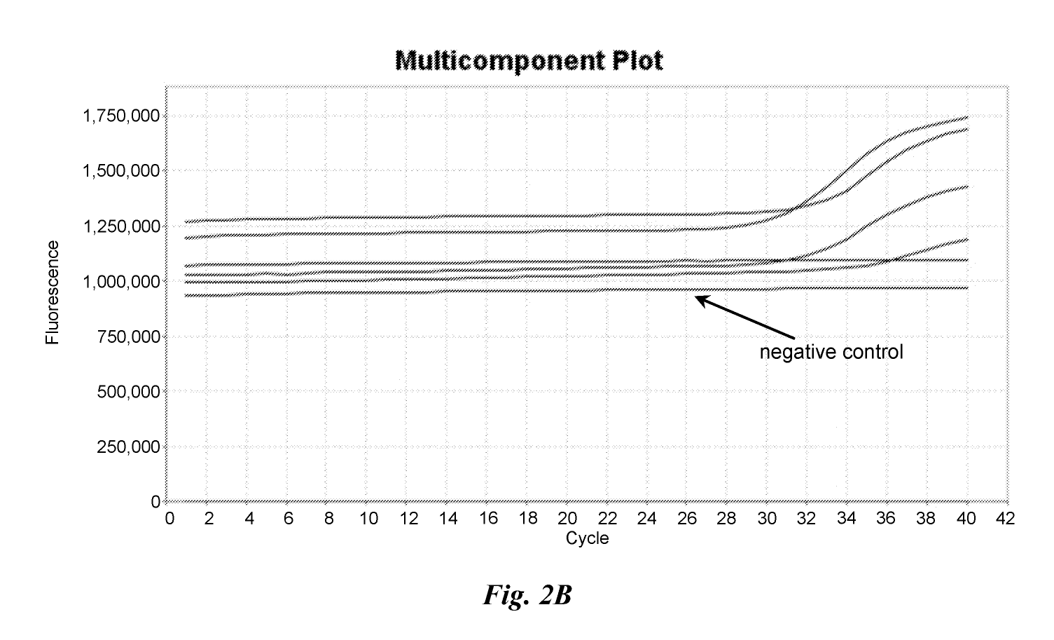

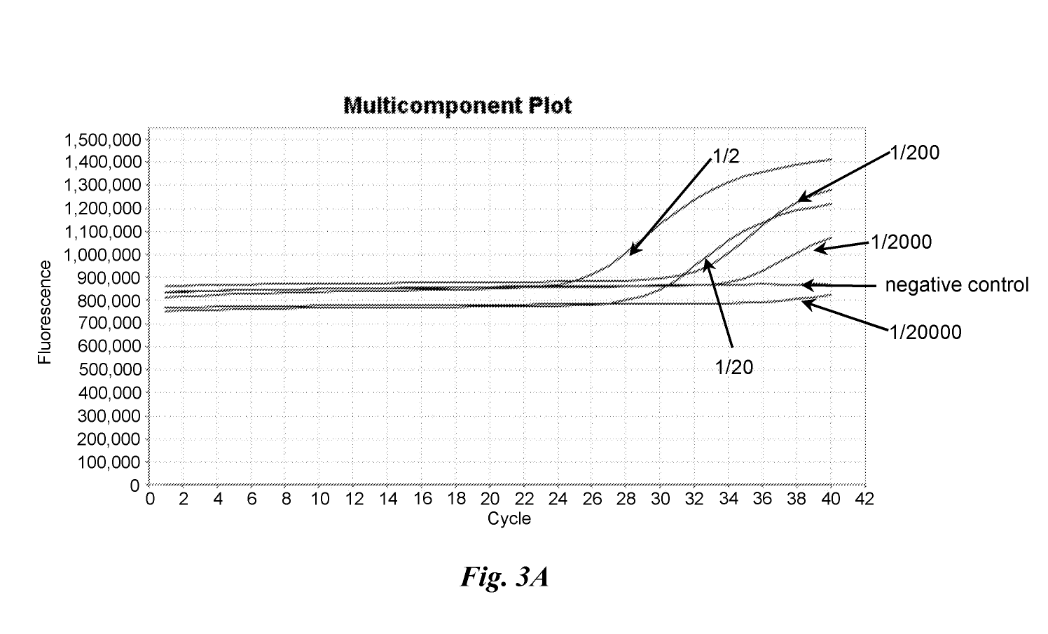

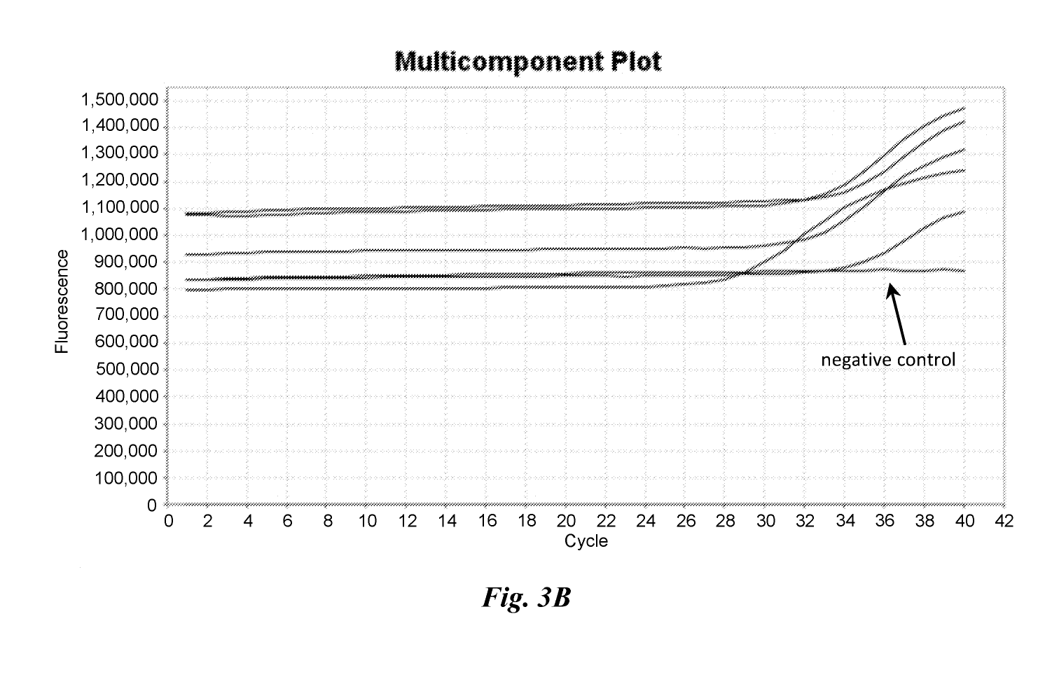

Amplification results for H3N2 are graphically depicted in FIG. 2 (FIG. 2A corresponds to the various viral standard samples, FIG. 2B corresponds to the biological samples) and amplification results for H1N1 (2007) are graphically depicted in FIG. 3 (FIG. 3A corresponds to the various viral standard samples, FIG. 3B corresponds to the biological samples). Recorded fluorescence of the FAM dye is plotted against the number of cycles.

As shown in FIG. 2A, each of the H3N2 viral standard samples showed detectable signal over the negative control, with Ct values ranging from 18 to 32. As shown in FIG. 2B, each of the viral H3N2 biological samples showed detectable signal over the negative control, with Ct values ranging from 29-35.

As shown in FIG. 3A and with the exception of the 1/20000 dilution, each of the H1N1 (2007) viral standard samples showed detectable signal over the negative control, with Ct values ranging from 24-35. As shown in FIG. 3B, each of the H1N1 (2007) biological samples showed detectable signal over the negative control, with Ct values ranging from 28-35.

In general, the data shown in FIGS. 2A and 2B and FIGS. 3A and 3B indicate that the tested viruses may be detected, via amplified DNA product, with good sensitivity, at concentrations as low as 50 IU/mL and over a 4-log concentration range with cycle threshold values of no more than about 40. Moreover, data also indicate that detection of viral RNA obtained from a biological sample obtained from a subject may also be detected in a similar fashion.

### Example 2: Amplification and Detection of Viral Nucleic Acid in Different Buffer Systems

Amplification and detection experiments were performed to compare results obtained from using different buffer systems for amplification. A set of experiments was conducted for two different buffer systems, S1 and S2. The S1 buffer included a zwitterionic buffering agent and BSA, and the S2 buffer included zwitterionic buffering agent and Sodium hydroxide. Experiments for each buffer were completed using a set of H5N1 influenza virus standard samples obtained as serial dilutions of a stock solution comprising the virus. The concentration H5N1 was 10⁶ IU/mL. Dilutions of ½, 1/20, 1/200, 1/2000, 1/20000, 1/200000, and a negative control were subject to amplification.

Five microliters of each sample were combined in a 25 μL reaction tube with reagents necessary to conduct reverse transcription of the viral RNA and reagents necessary to complete amplification of the complementary DNA obtained from reverse transcription (e.g., parallel nucleic acid amplification). The reagents necessary to conduct reverse transcription and DNA amplification included reverse transcriptases, a DNA polymerase, dNTPs, and the appropriate S1 or S2 buffer. Moreover, the reaction tubes also included a TaqMan probe comprising a FAM dye for detection of amplified DNA product. To generate amplified DNA product, each reaction mixture incubated according to a protocol of denaturing and elongation conditions comprising 5 min at 95° C., followed by 20 min at 45° C., followed by 2 min at 95° C., and followed by 40 cycles of 5 seconds at 95° C. and 30 seconds at 55° C. in a real-time PCR thermocycler. Detection of amplified product occurred during incubations.

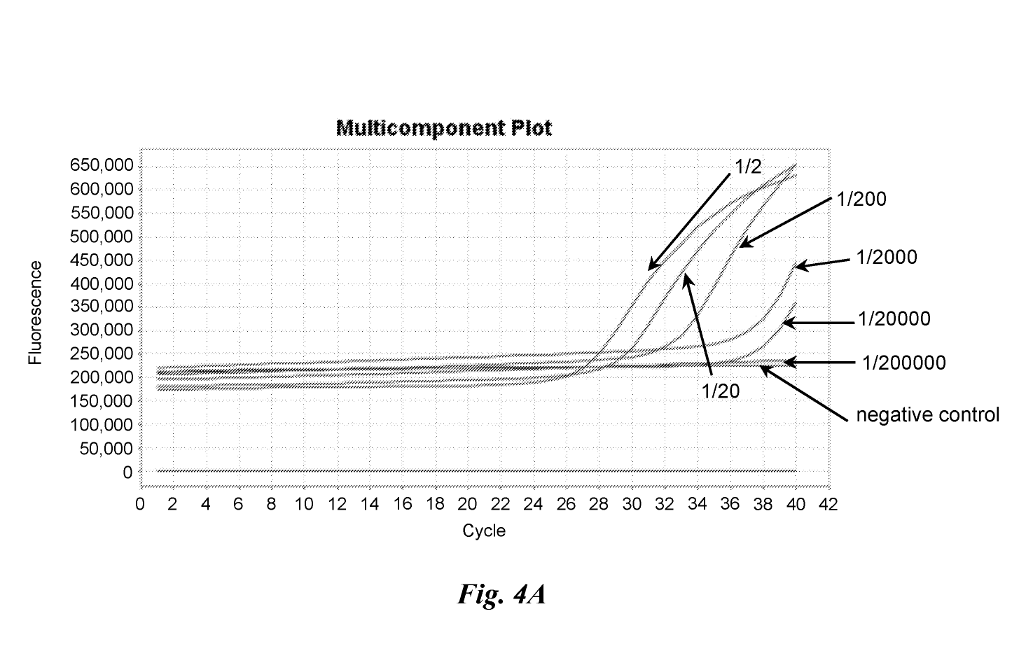

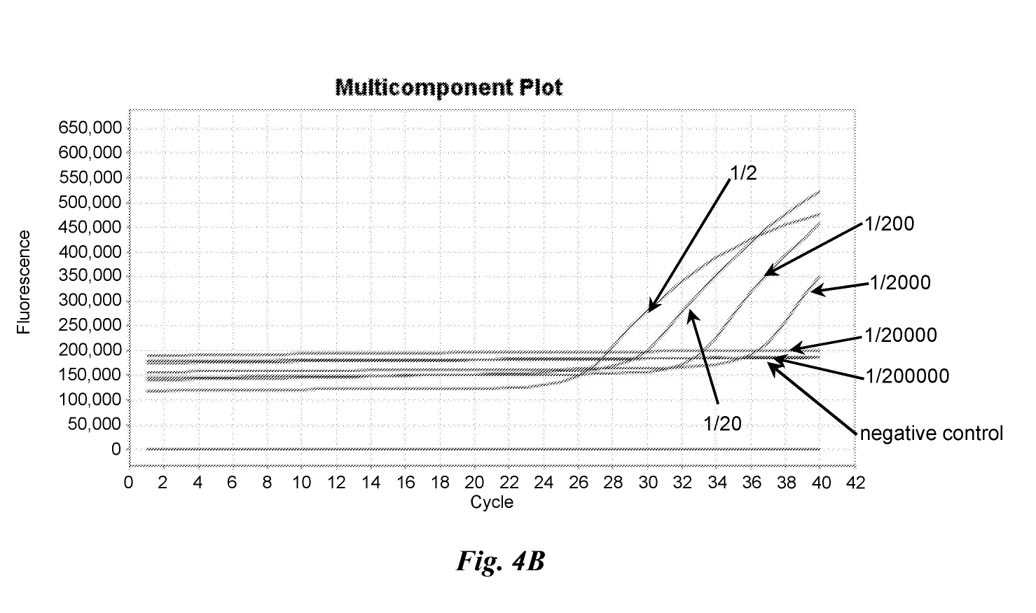

Amplification results for buffer system S1 are graphically depicted in FIG. 4A and amplification results for buffer system S2 were graphically depicted in FIG. 4B. Recorded fluorescence of the FAM dye is plotted against the number of cycles.

As shown in FIG. 4A each of the viral standard samples amplified in buffer system S1 showed detectable signal over the negative control, with Ct values ranging from 25 to 36. As shown in FIG. 4B, each of the viral standard samples amplified in buffer S2 showed detectable signal over the negative control, with Ct values ranging from 25-35.

In general, the data shown in FIGS. 4A and 4B indicate that the tested virus may be detected, via amplified DNA product, with good sensitivity, at concentrations as low as 50 IU/mL and over a 5-log concentration range with cycle threshold values of no more than about 40. Moreover, data also indicate that similar amplification results can be obtained with different buffer systems.

### Example 3: Amplification and Detection of Hepatitis B Virus (HBV) in Plasma Samples

Amplification experiments were performed to determine the robustness of an amplification method to detect target nucleic acid in a biological sample. Diluted human blood plasma samples comprising hepatitis B virus (HBV) at various concentrations (e.g., 50 infective units per milliliter (IU/mL), 200 IU/mL, 2000 IU/mL, 20000 IU/mL) were each subject to amplification reactions. HBV is a DNA virus that replicates via an RNA intermediate. HBV is detectable via direct PCR of the DNA virus. Multiple samples (n=2-4) were tested for each concentration in additional to multiple samples of a negative control (e.g., plasma not comprising HBV).

2.5 μL of each sample with reagents necessary to conduct reverse transcription of the RNA and reagents necessary to complete amplification of the complementary DNA obtained from reverse transcription (e.g., parallel nucleic acid amplification) in a 50 μL reaction tube to obtain a reaction mixture. The reagents necessary to conduct reverse transcription and DNA amplification were supplied as a commercially available pre-mixture (e.g., Qiagen One-Step RT-PCR or One-Step RT-qPCR kit) comprising reverse transcriptases (e.g., Sensiscript and Omniscript transcriptases), a DNA Polymerase (e.g., HotStarTaq DNA Polymerase), and dNTPs. Moreover, the mixture also included a TaqMan probe comprising a FAM dye for detection of amplified DNA product. Also, the reaction mixture included a zwitterionic buffering agent and a uracil-DNA glycosylase (UNG) enzyme to prevent inhibitory effects of amplification inhibitors found in plasma. Each reaction mixture was incubated according to a protocol of denaturing and elongation conditions comprising 1 min at 94° C., followed by 10 min at 50° C., followed by 2 min at 94° C., and followed by 50 cycles of 5 seconds at 94° C. and 35 seconds at 58° C. in a real-time PCR thermocyler. Detection of amplified product occurred during incubations.

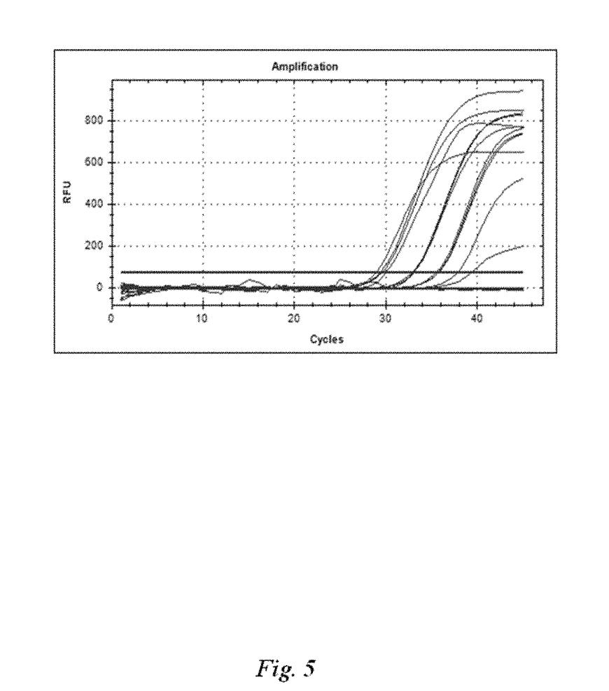

Amplification results are graphically depicted in FIG. 5 and determined Ct values tabulated in Table 1. Recorded relative fluorescence units (RFU) of the FAM dye is plotted against the number of cycles in FIG. 5. As shown in FIG. 5 and Table 1, HBV may be detected at each concentration tested with cycle threshold values ranging from 28.99 to 39.39. In general, higher concentration samples corresponded to lower cycle threshold values.

In general, the data shown in FIG. 5 and Table 1 indicate HBV may be detected, via amplified DNA product, with good sensitivity, at concentrations as low as 50 IU/mL (the lowest tested) with cycle threshold values of no more than about 40. While the highest concentration tested (20000 IU/mL) was 400 times more concentrated than the lowest concentration tested (50 IU/mL), cycle threshold values were only about 25% higher for lower concentrations, indicating that the amplification scheme was generally robust.

TABLE 1
Ct Results from Experiments in Example 3

| Sample # | IU/mL | Ct |
|---|---|---|
| 1 | 2000 | 33.09 |
| 2 | 50 | 39.39 |
| 3 | 2.00E+04 | 29 |
| 4 | 2000 | 32.97 |
| 5 | 200 | 35.51 |
| 6 | 2000 | 33.07 |
| 7 | 2.00E+04 | 30.03 |
| 8 | 200 | 35.78 |
| 9 | 50 | 37.91 |
| 10 | 2.00E+04 | 29.37 |
| 11 | 200 | 35.73 |
| 12 | 2.00E+04 | 28.99 |

### Example 4: Pre-Heating a Biological Sample Prior to Amplification of Nucleic Acid in the Biological Sample and Series of Amplification Reactions

Amplification experiments were conducted to determine the effect of pre-heating a biological sample on detection sensitivity and also to determine the effect of using multiple series of amplification reactions on detection sensitivity.

Twenty 25 μL reaction mixtures were prepared, with each reaction mixture comprising 1 μL of a pathogenic species, reagents necessary to complete appropriate nucleic acid amplification reactions (e.g., reverse transcription and DNA amplification for RNA species, and DNA amplification for DNA species), and a TaqMan probe comprising a FAM dye. Four of the reaction mixtures contained H1N1 (2007) (i.e., an RNA virus four of the reaction mixtures contained H3N2 (i.e., an RNA virus), four of the reaction mixtures contained H1N1 (2009), four of the reaction mixtures contained tuberculosis (TB) (i.e., a bacterial sample), and four of the reaction mixtures contained Aleutian disease virus (ADV) (i.e., a DNA virus). H1N1 (2007), H1N1 (2009), H3N2, and ADV pathogenic species were from oropharyngeal swabs obtained from subjects. TB was obtained from a bacterium stock.

Various combinations of pre-heating and amplification protocols were utilized and are summarized in Table 2. For the first reaction mixture for each pathogenic species, the pathogenic species was pre-heated 10 min at 95° C. prior to being added to the reaction mixture. After addition of the pathogenic species to the reaction mixture, the reaction mixture was incubated according to a protocol of denaturing and elongation conditions comprising 2 minutes at 95° C. followed by 40 cycles of 5 seconds at 95° C. and 30 seconds at 55° C. in a real-time PCR thermocycler. Detection of amplified product occurred during incubations. These reaction mixtures are referred to as PH-1 mixtures.

For the second reaction mixture for each pathogenic species, the pathogenic species was pre-heated 30 min at 50° C. prior to being added to the reaction mixture. After addition of the pathogenic species to the reaction mixture, the reaction mixture was incubated according to a protocol of denaturing and elongation conditions comprising 2 minutes at 95° C. followed by 40 cycles of 5 seconds at 95° C. and 30 seconds at 55° C. in a real-time PCR thermocycler. Detection of amplified product occurred during incubations. These reaction mixtures are referred to as PH-2 mixtures.

For the third reaction mixture for each pathogenic species, the pathogenic species was not pre-heated prior to being added to the reaction mixture. These reaction mixtures incubated according to a protocol of denaturing and elongation conditions comprising 1 min at 95° C., followed by 10 minutes at 55° C., followed by 2 minutes at 95° C., followed by 40 cycles of 5 seconds at 95° C. and 30 seconds at 55° C. in a real-time PCR thermocycler. Detection of amplified product occurred during incubations. These reaction mixtures are referred to as PTC-1 mixtures.

For the fourth reaction mixture for each pathogenic species, the pathogenic species was not pre-heated prior to being added to the reaction mixture. These reaction mixtures were subjected to a protocol comprising a plurality of series of amplification reactions, with each series comprising multiple cycles of denaturing and elongation conditions. Reaction mixtures were incubated according to such a protocol comprising 1 minute at 95° C., followed by 10 cycles of Series 1 (95° C. for 5 seconds, 20 seconds of 60-50° C., stepping down 1° C./cycle, and 60° C. for 10 seconds), followed by 2 minutes 95° C. for 2 minutes, followed by 40 cycles of Series 2 (95° C. for 5 seconds, 55° C. for 30 seconds) in a real-time PCR thermocycler. Series 1 and Series 2 differ in their elongation temperature and elongation duration. Detection of amplified product occurred during incubations. These reaction mixtures are referred to as PTC-2 mixtures.

TABLE 2
Experimental Conditions of Example 4

| Reaction | |
|---|---|
| Mixture | |
| Type | Protocol |
| PH-1 | 95° C. 10 minute preheating on pathogenic species before |
| | adding to the reaction mixture, then 95° C. for 2 minutes, |
| | (95° C. for 5 seconds, 55° C. for 30 seconds) × 40 cycles |
| PH-2 | 50° C. 30 minute preheating on pathogenic before adding to |
| | the reaction mixture, then 95° C. for 2 minutes, (95° C. for 5 |
| | seconds, 55° C. for 30 seconds) × 40 cycles |
| PTC-1 | 95° C. for 1 minute, 55° C. for 10 minutes, then 95° C. for |
| | 2 minutes, (95° C. for 5 seconds, 55° C. for 30 seconds) × 40 |
| | cycles |
| PTC-2 | 95° C. for 1 minute, (95° C. for 5 seconds, 60-50° C., |
| | stepping down 1° C./cycle, for 20 seconds, 60° C. for 10 |
| | seconds) × 10 cycles, then 95° C. for 2 minutes, (95° C. for 5 |
| | seconds, 55° C. for 30 seconds) × 40 cycles |

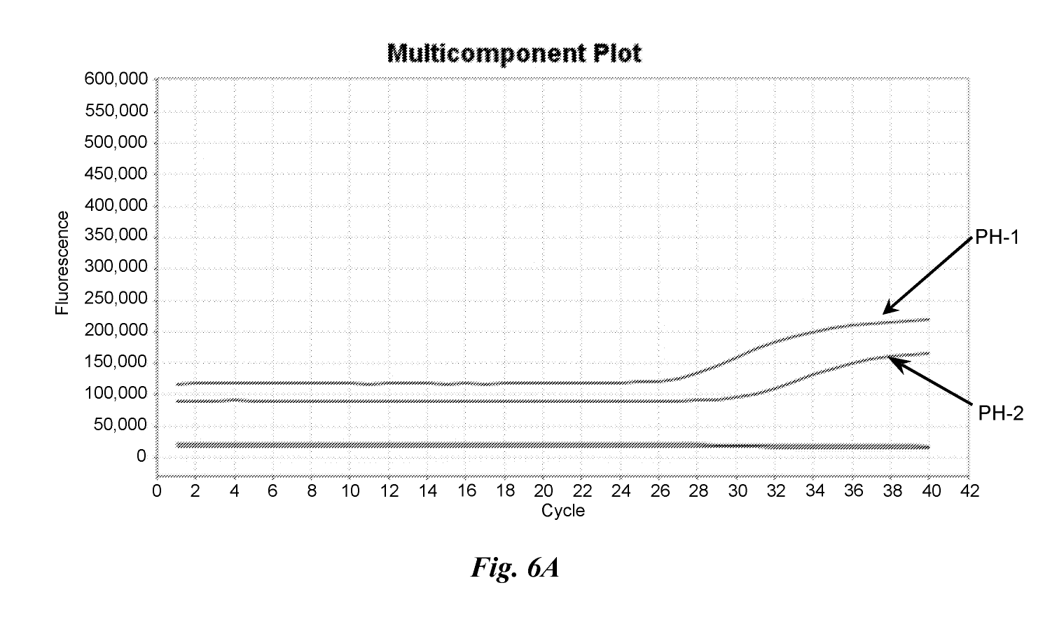

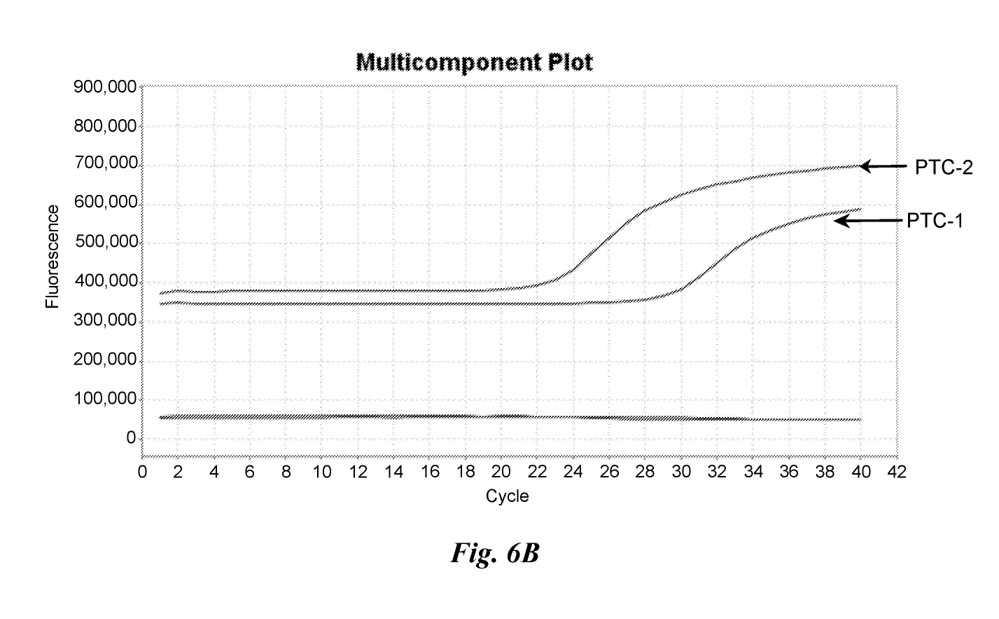

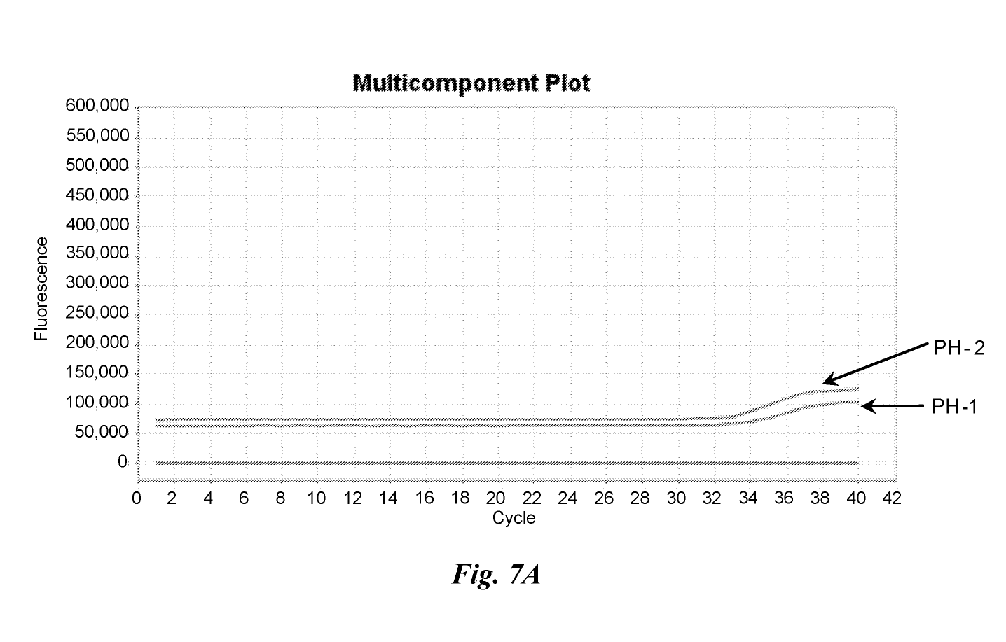

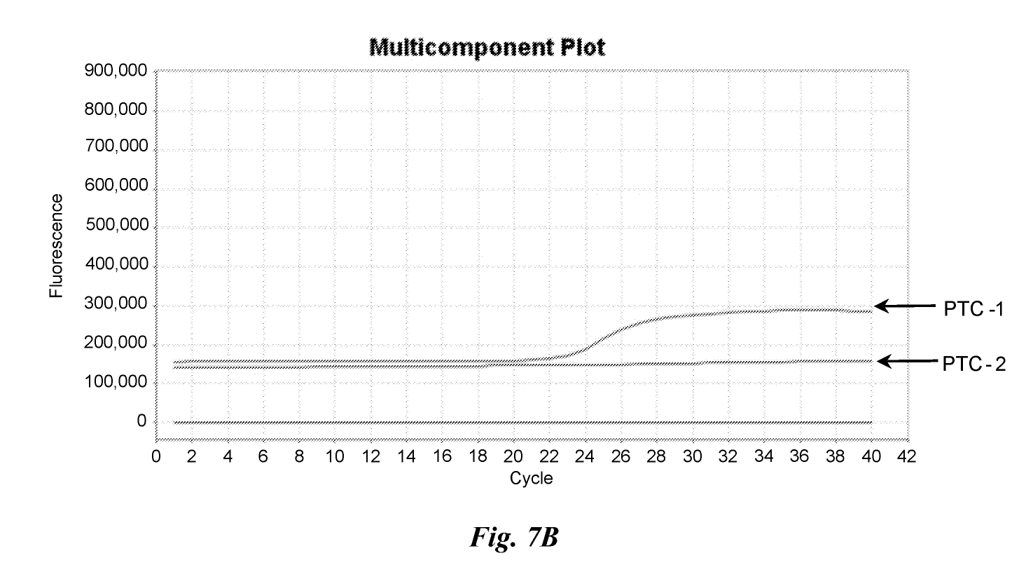

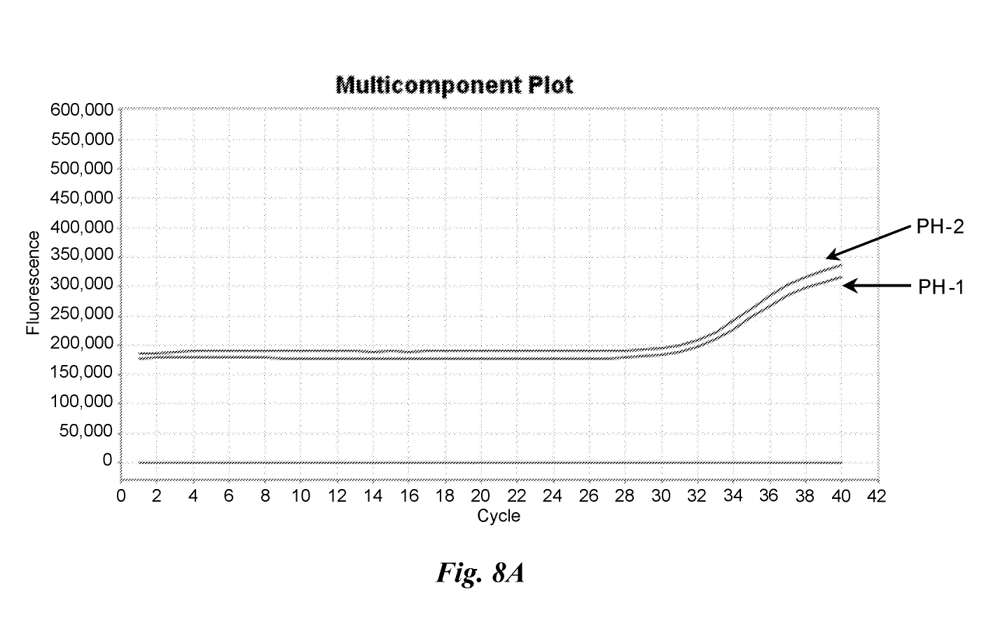

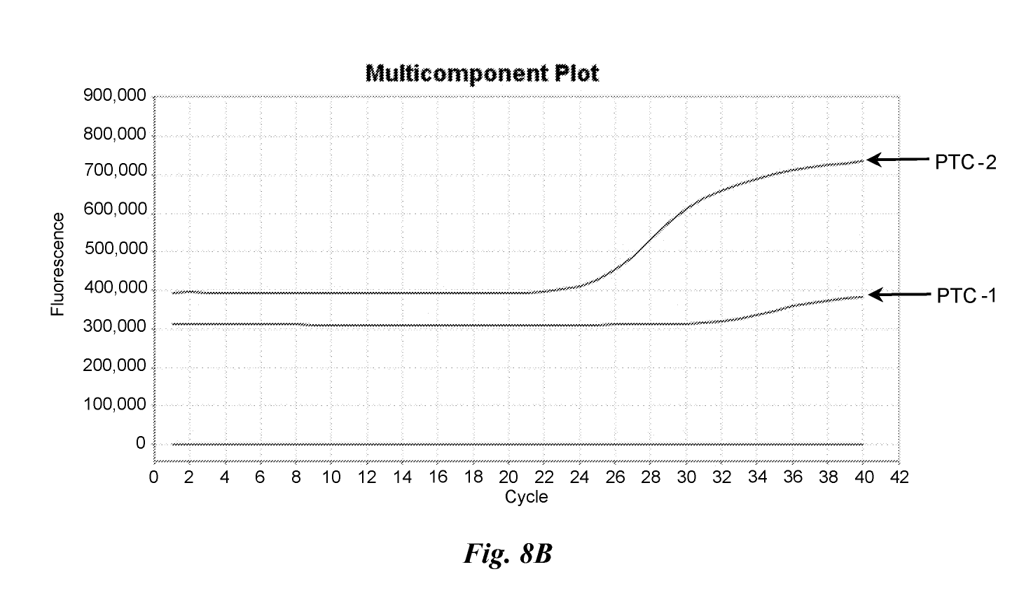

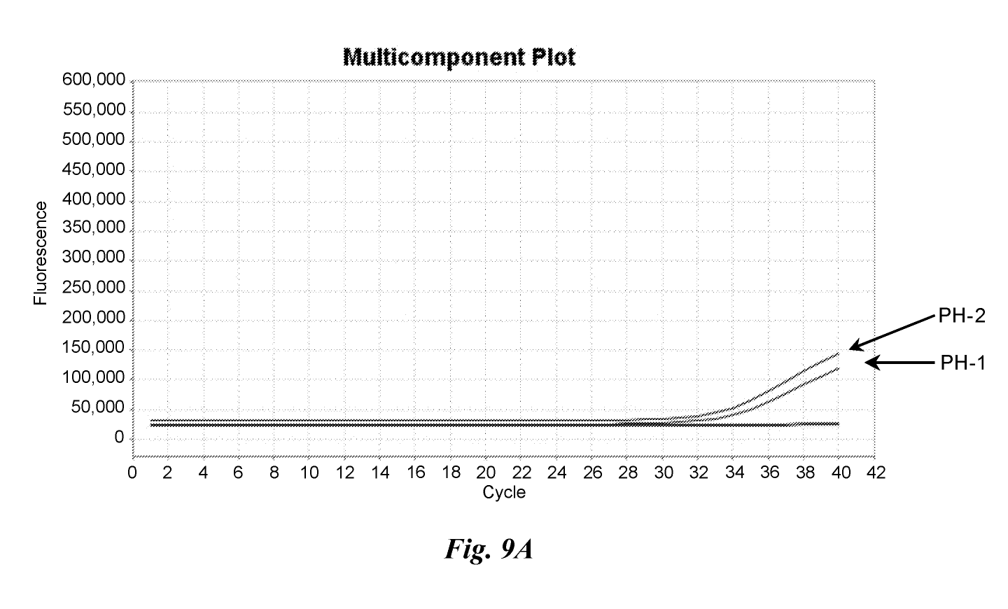

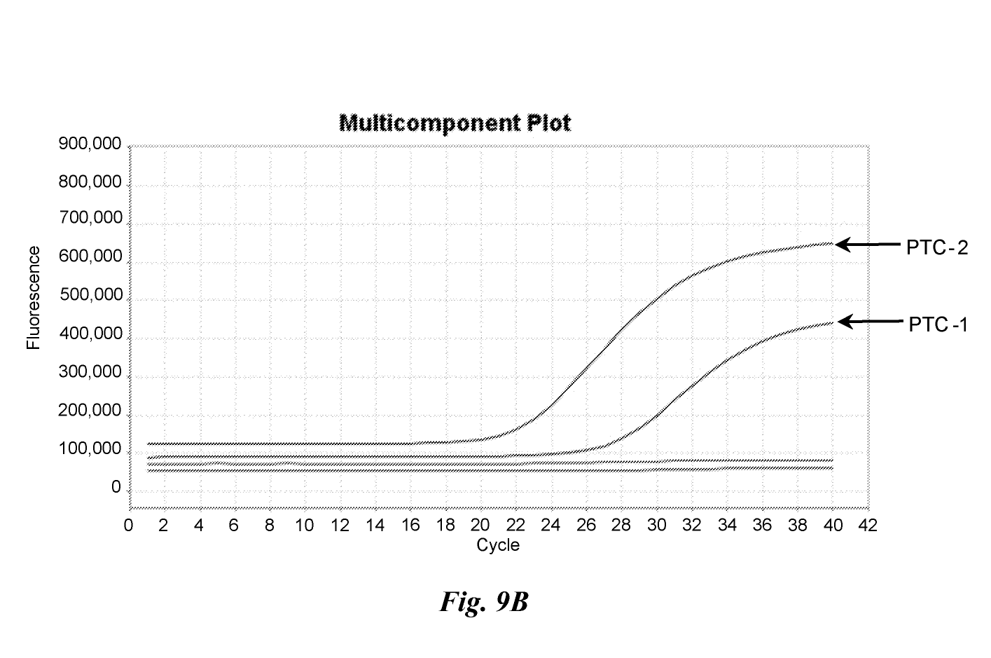

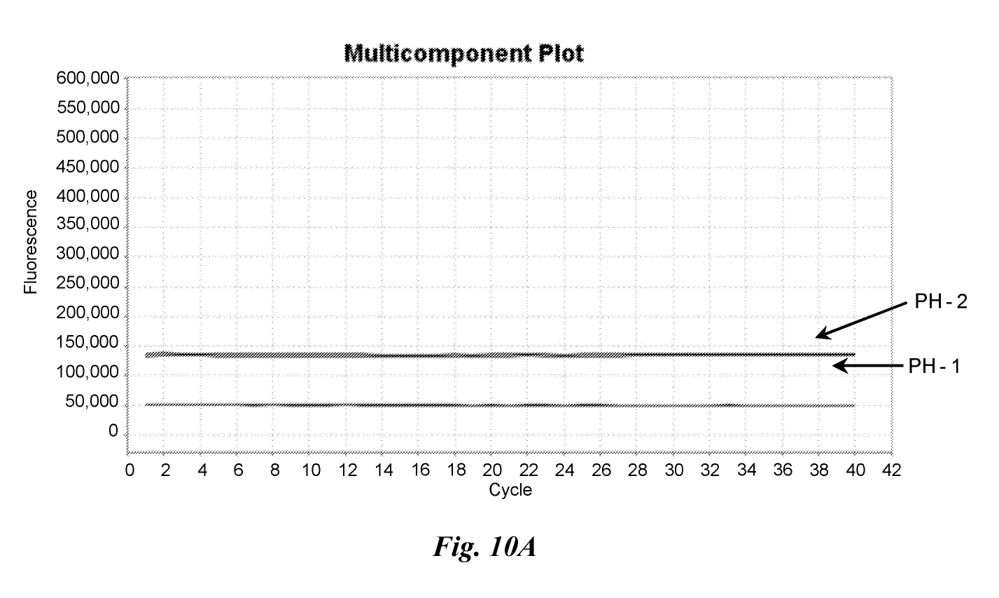

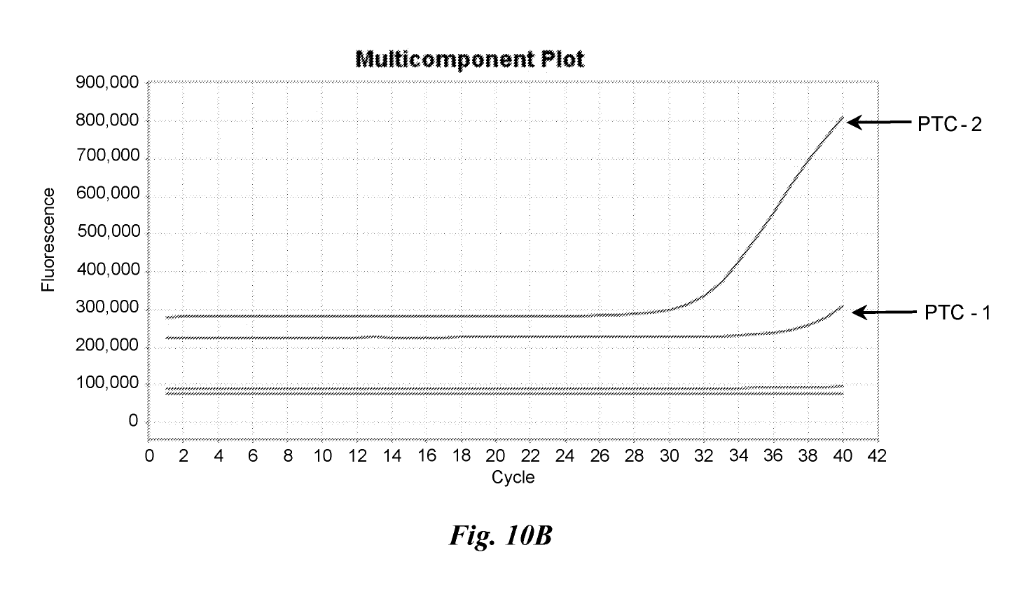

Results from each pathogenic species are graphically depicted in FIGS. 6A and 6B (H1N1 (2007), FIGS. 7A and 7B (H3N2), FIGS. 8A and 8B (H1N1 (2009)), FIGS. 9A and 9B (TB), and FIGS. 10A and 10B (ADV). FIGS. 6A, 7A, 8A, 9A and 10A represent results obtained for reaction mixtures PH-1 and PH-2, whereas FIGS. 6B, 7B, 8B, 9B and 10B represent results obtained for reaction mixture PTC-1 and PTC-2. Ct values determined for each experiments are summarized in Table 3. Ct values may not be determined for PH-1 and PH-2 ADV reaction mixtures, commensurate with the data shown in FIG. 10A.

According to data shown in Table 3, Ct values between were fairly similar between PH-1 and PH-2 reaction mixtures, indicating that a pathogenic species (or biological sample comprising a pathogenic species) may be pre-heated at a range of conditions to obtain similar detection sensitivity. Moreover, PTC-1 reaction mixtures had Ct values similar to those determined for PH-1 and PH-2 reaction mixtures. PTC-1 and PH-1/PH-2 protocols were similar, except that PTC-1 did not include a pre-heating step. Thus, a comparison of PTC-1 data with PH-1/PH-2 data indicates that pre-heating of a pathogenic species prior to providing it to a reaction mixture may not be necessary for obtaining results with good sensitivity. However, in some cases with TB and ADV samples, pre-heating can be even worse than without pre-heating.

However, for all pathogenic species tested, PTC-2 Ct values were lower than any of PH-1, PH-2, or PTC-1. A comparison of PTC-1 and PTC-2 data indicate that subjecting reaction mixtures to a multiple series of amplification reactions, with each series comprising multiple cycles of denaturing and elongation conditions, may improve detection sensitivity.

TABLE 3
Ct Results from Experiments in Example 4

| | | PH-1 | PH-2 | PTC-1 | PTC-2 |
|---|---|---|---|---|---|
| Type | Sample | (Ct) | (Ct) | (Ct) | (Ct) |
| RNA virus | H1N1(2007) | 27 | 30 | 28 | 22 |
| RNA virus | H3N2 | 34 | 33 | 32 | 23 |
| RNA virus | H1N1(2009) | 32 | 32 | 32 | 24 |
| DNA bacteria | TB | 34 | 32 | 26 | 20 |
| DNA virus | ADV | — | — | 36 | 30 |

### Example 5: Multiplexing Samples

Amplification and detection experiments were performed to benchmark various amplification protocols and to determine whether multiplexing may be achieved. Biological samples comprising RNA (e.g., H1N1 (2007), H1N1 (2009), H3N2) or DNA (e.g., ADV, human bocavirus (HBoV) viral pathogens or DNA bacterial pathogens (e.g., TB) were subject to various amplification conditions. Each biological sample was obtained directly from a subject via an oropharyngeal swab, except for TB samples which were from a bacterium stock. One microliter of each sample was combined in a 25 μL reaction tube with reagents necessary to conduct nucleic acid amplification and to detect amplified product as described herein to obtain a reaction mixture.

To assess the multiplexing capabilities of an amplification protocol, three reaction mixtures, each comprising one of H3N2, ADV, or a mixture of H3N2 and ADV were incubated according to an amplification protocol comprising 2 min at 94° C., 20 min at 45° C., 1 min at 94° C., followed by 50 cycles of 5 seconds at 94° C. and 35 seconds at 55° C. in a real time PCR thermocycler. Detection of amplified product occurred during incubations.

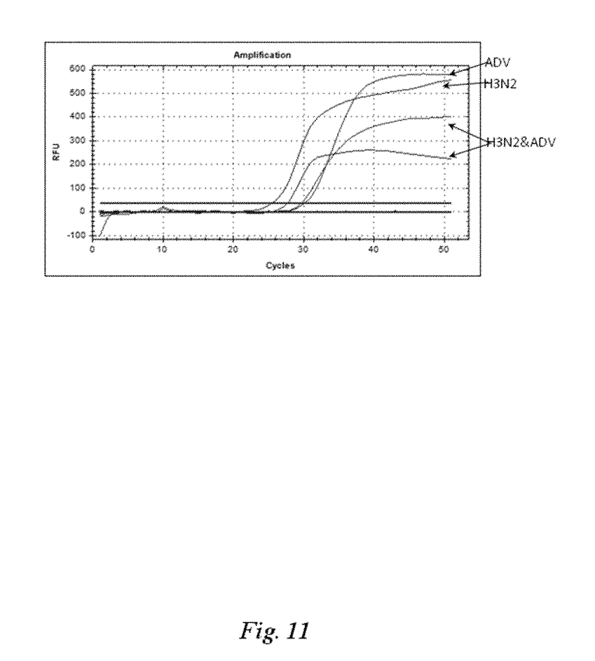

Results of the experiments are graphically depicted in FIG. 11 and shown below in Table 4. As shown in FIG. 11, both H3N2 and TB may be detected similarly when in combination or in the absence of the other. In the absence of ADV, a Ct value of 26.03 was recorded for the H3N2 reaction mixture and in the absence of H3N2, a Ct value of 30.5 was recorded for the ADV reaction mixture. When both of H3N2 and ADV were combined into a single reaction mixture, Ct values of 26 (H3N2) and 30 (ADV) were obtained. Ct values were nearly identical for the combined reaction mixture when compared to the single component reaction mixtures. Results indicate that multiplexing is achievable with good sensitivity and that both RNA and DNA species can be detected.

TABLE 4
Results from H3N2 and ADV Multiplexing
Experiment in Example 5

| Type | Sample | Ct |
|---|---|---|
| RNA virus | H3N2 | 26.03 |
| DNA virus | ADV | 30.5 |
| RNA & DNA virus | H3N2 & ADV | 26(H3N2) & 30(ADV) |

In another experiment to assess the multiplexing capabilities of an amplification protocol, three reaction mixtures, each comprising one of H3N2, TB, or a mixture of H3N2 and TB were incubated according to an amplification protocol comprising 2 min at 95° C., followed by 40 cycles of 5 seconds at 95° C. and 30 seconds at 55° C. in a real time PCR thermocycler. Detection of amplified product occurred during incubations.

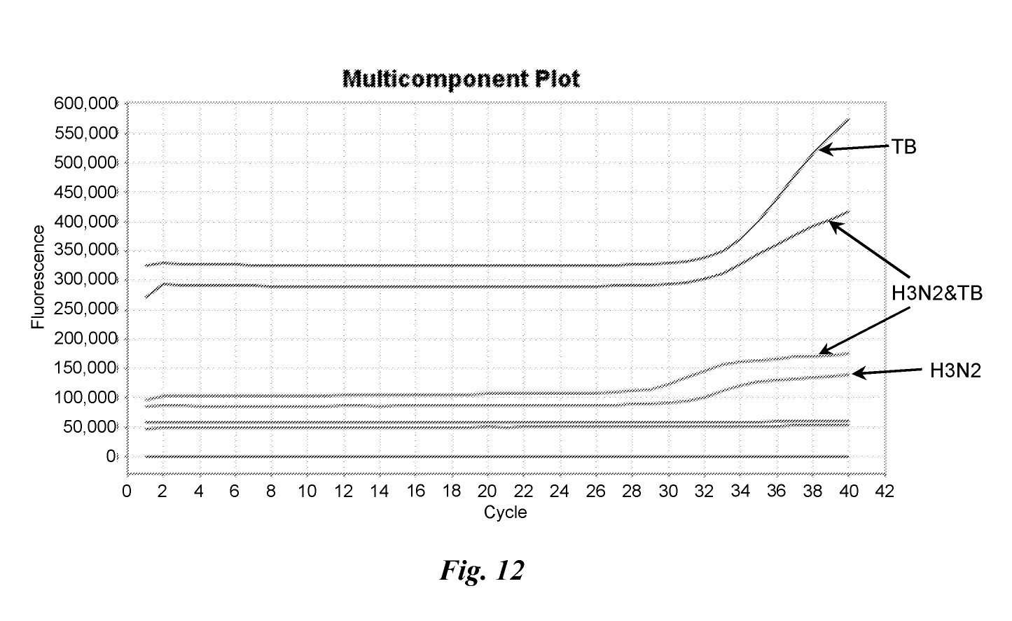

Results of the experiments are graphically depicted in FIG. 12 and shown below in Table 5. As shown in FIG. 12, both H3N2 and TB may be detected similarly when in combination or in the absence of the other. In the absence of TB, a Ct value of 32 was recorded for the H3N2 reaction mixture and in the absence of H3N2, a Ct value of 32 was recorded for the TB reaction mixture. When both of H3N2 and TB were combined into a single reaction mixture, Ct values of 29 (H3N2) and 30 (TB) were obtained. Ct values were similar for the combined reaction mixture when compared to the single component reaction mixtures. Results indicate that multiplexing is achievable with good sensitivity and that both RNA and DNA species can be detected in a multiplexing scheme.

TABLE 5
Results from H3N2 and TB Multiplexing Experiment in Example 5

| Type | Sample | Ct |
|---|---|---|
| RNA virus | H3N2 | 32 |
| DNA virus | TB | 32 |
| RNA & DNA virus | H3N2 & TB | 29(H3N2) & 30(TB) |

### Example 6: Benchmarking Multiple Series of Amplification Reactions

Amplification and detection experiments were performed to benchmark various amplification protocols comprising multiple series of amplification reactions. Biological samples comprising RNA (e.g., H1N1 (2007), H1N1 (2009), H3N2) or DNA (e.g., ADV, human bocavirus (HBoV) viral pathogens or DNA bacterial pathogens (e.g., TB) were subject to various amplification conditions. Each biological sample was obtained directly from a subject via an oropharyngeal swab, except for TB samples which were from a bacterium stock. One microliter of each sample was combined in a 25 μL reaction tube with reagents necessary to conduct nucleic acid amplification and to detect amplified product as described herein to obtain a reaction mixture.

In one set of experiments, amplification mixtures were subjected to an amplification protocol comprising two series of amplification reactions, each series comprising different denaturation and elongation conditions. Six reaction mixtures (two comprising H3N2, two comprising ADV, two comprising HBoV) were incubated according to an amplification protocol comprising 1 second at 94° C., followed by 11 cycles of Series 1 (1 second at 94° C. and 10 seconds at 45° C.), followed by 1 minute at 95° C., followed by 40 cycles of Series 2 (5 seconds at 95° C. and 30 seconds at 55° C.) in a real time PCR thermocycler. Detection of amplified product occurred during incubations.

Results of the experiments are shown below in Table 6. As shown in Table 6, determined Ct values ranged from 8.35 to 23. Results indicate that protocols comprising multiple series of amplification reactions can be useful in achieving good sensitivity. Moreover, results also indicate that both RNA and DNA species can be detected with protocols comprising multiple series of amplification reactions.

TABLE 6
Results from H3N2, ADV, and HBoV Experiment in Example 6

| Type | Sample | Ct |
|---|---|---|
| RNA virus | H3N2-1 | 17 |
| RNA virus | H3N2-2 | 20 |
| DNA virus | ADV-1 | 18.8 |
| DNA virus | ADV-2 | 23 |
| DNA virus | HBoV-1 | 8.35 |
| DNA virus | HBoV-2 | 18.37 |

In another set of experiments, amplification mixtures were subjected to an amplification protocol comprising three series of amplification reactions, the series differing from the others with respect to their denaturation and/or elongation condition. Five reaction mixtures (one comprising sH1N1 (2007), one comprising H3N2, one comprising pH1N1 (2009), one comprising ADV, and one comprising TB) were incubated according to an amplification protocol comprising 1 minute at 94° C., followed by 5 cycles of Series 1 (5 seconds at 94° C. and 30 seconds at 60-50° C. stepped down 1° C./cycle), followed 5 cycles of Series 2 (5 seconds at 94° C. and 30 seconds at 50° C.), followed by 2 minutes at 95° C., followed by 40 cycles of Series 3 (5 seconds at 95° C. and 30 seconds at 55° C.) in a real time PCR thermocycler. Detection of amplified product occurred during incubations.

Results of the experiments are shown below in Table 7. As shown in Table 7, determined Ct values ranged from 20 to 30. Results indicate that protocols comprising multiple series of amplification reactions can be useful in achieving good sensitivity. Moreover, results also indicate that both RNA and DNA species can be detected with protocols comprising multiple series of amplification reactions.

TABLE 7
Results from sH1N1(2007), H3N2, pH1N1(2009),
ADV, and TB Experiment in Example 6

| Type | Sample | Ct |
|---|---|---|
| RNA virus | sH1N1(2007) | 22 |
| RNA virus | H3N2 | 23 |
| RNA virus | pH1N1(2009) | 24 |
| DNA virus | ADV | 30 |
| DNA bacteria | TB | 20 |

### Example 7: Benchmarking Multiple Series of Amplification Reactions

Amplification and detection experiments were performed to benchmark various amplification protocols comprising multiple series of amplification reactions. Biological samples comprising H3N2 were subject to various amplification conditions. Each biological sample was obtained directly from a subject via an oropharyngeal swab. One microliter of each sample was combined in a 25 μL reaction tube with reagents necessary to conduct nucleic acid amplification and to detect amplified product as described herein to obtain a reaction mixture.

Amplification mixtures were subjected to amplification protocols, some comprising one of three different first series of amplification reactions and the same second series, the three first series comprising different denaturation and elongation conditions than the second series. Each of first series and the second series comprised multiple cycles. Another experiment was conducted without a first series, comprising only the second series. In a real time PCR thermocycler, each of four reaction mixtures comprising H3N2 were incubated according to one of the amplification protocols shown below in Table 8:

TABLE 8
Experimental Protocols in Example 7

| Reaction | |
|---|---|
| Mixture | Protocol |
| 1 | 94° C. for 1 minute, (Series 1A -- 94° C. for 1 second, 45° |
| | C. for 2 minutes) × 5 cycles, 95° C. for 1 minute, (Series 2 -- |
| | 95° C. for 5 seconds, 55° C. for 30 seconds) × 50 cycles |
| 2 | 80° C. for 2 minutes, (Series 1B -- 80° C. for 1 second, 45° |
| | C. for 2 minutes) × 5 cycles, 95° C. for 1 minute, (Series 2 -- |
| | 95° C. for 5 seconds, 55° C. for 30 seconds) × 50 cycles |
| 3 | 80° C. for 2 minutes, 45° C. for 30 minutes, 95° C. for 1 |
| | minute, (Series 2 -- 95° C. for 5 seconds, 55° C. for 30 |
| | seconds) × 50 cycles |
| 4 | 94° C. for 1 second, (Series 1C -- 94° C. for 1 second, 45° |
| | C. for 30 seconds) × 50 cycles, (Series 2 -- 95° C. for 5 |
| | seconds, 55° C. for 30 seconds) × 50 cycles |

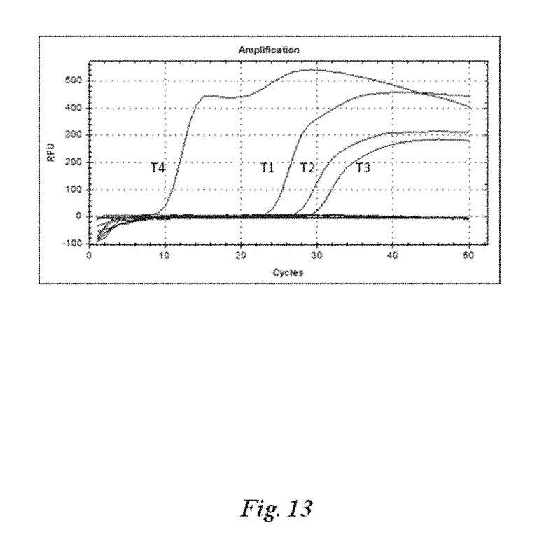

Results of the experiments are graphically depicted in FIG. 13 and tabulated below in Table 9. As shown in FIG. 13, reaction mixture 3 had the highest Ct value (28.59). The others comprising multiple series had lower values ranging from 8.5 to 26.5. Results indicate that protocols comprising multiple series of amplification reactions can be useful in achieving good sensitivity. Moreover, results also indicate that protocols comprising multiple series of amplification reactions may achieve better sensitivity when compared to protocols with only a single series.

TABLE 9
Experimental Results of Example 7

| Reaction | |
|---|---|
| Mixture | Ct |
| 1 | 22.97 |
| 2 | 26.5 |
| 3 | 28.59 |
| 4 | 8.5 |

### Example 8: Benchmarking Multiple Series of Amplification Reactions

Amplification and detection experiments were performed to benchmark various amplification protocols comprising multiple series of amplification reactions. Biological samples comprising H3N2 were subject to various amplification conditions. Each biological sample was obtained directly from a subject via an oropharyngeal swab. One microliter of each sample was combined in a 25 μL reaction tube with reagents necessary to conduct nucleic acid amplification and to detect amplified product as described herein to obtain a reaction mixture.

Amplification mixtures were subjected to amplification protocols, some comprising one of six first series of amplification reactions and the same second series, the six first series comprising different denaturation and elongation conditions than the second series. Another six experiments were conducted without a first series. In a real time PCR thermocycler, each of twelve reaction mixtures comprising H3N2 were incubated according to one of the amplification protocols shown below in Table 10:

TABLE 10
Experimental Protocols in Example 8

| Reaction | |
|---|---|
| Mixture | Protocol |
| 1 | 95° C. for 3 minutes, 45° C. for 5 minutes, 95° C. for 1 minute, (Series 2 -- 95° C. for 5 |
| | seconds, 55° C. for 30 seconds) × 40 cycles |
| 2 | 95° C. for 10 minutes, 45° C. for 5 minutes, 95° C. for 1 minute, (Series 2 -- 95° C. for 5 |
| | seconds, 55° C. for 30 seconds) × 40 cycles |
| 3 | 95° C. for 3 minutes, 45° C. for 20 minutes, 95° C. for 1 minute, (Series 2 -- 95° C. for 5 |
| | seconds, 55° C. for 30 seconds) × 40 cycles |
| 4 | 95° C. for 10 minutes, 45° C. for 20 minutes, 95° C. for 1 minute, (Series 2 -- 95° C. for 5 |
| | seconds, 55° C. for 30 seconds) × 40 cycles |
| 5 | 95° C. for 10 minutes, 45° C. for 3 minutes, 95° C. for 1 minute, (Series 2 -- 95° C. for 5 |
| | seconds, 55° C. for 30 seconds) × 40 cycles |
| 6 | 45° C. for 20 minutes, 95° C. for 1 minute, (Series 2 -- 95° C. for 5 seconds, 55° C. for 30 |
| | seconds) × 40 cycles |
| 7 | 94° C. for 2 minutes, (Series 1A -- 94° C. for 1 second, 45° C. for 10 seconds) × 10 cycles, |
| | 95° C. for 1 minute, (Series 2 -- 95° C. for 5 seconds, 55° C. for 30 seconds) × 50 cycles |
| 8 | 94° C. for 10 seconds, (Series 1B -- 94° C. for 1 second, 45° C. for 10 seconds) × 10 cycles, |
| | 95° C. for 1 minute, (Series 2 -- 95° C. for 5 seconds, 55° C. for 30 seconds) × 50 cycles |
| 9 | 94° C. for 2 minutes, (Series 1C -- 94° C. for 10 seconds, 45° C. for 20 seconds) × 10 |
| | cycles, 95° C. for 1 minute, (Series 2 -- 95° C. for 5 seconds, 55° C. for 30 seconds) × 50 |
| | cycles |
| 10 | 94° C. for 10 seconds, (Series 1D -- 94° C. for 10 seconds, 45° C. for 20 seconds) × 10 |
| | cycles, 95° C. for 1 minute, (Series 2 -- 95° C. for 5 seconds, 55° C. for 30 seconds) × 50 |
| | cycles |
| 11 | 94° C. for 2 minutes, (Series 1E -- 94° C. for 30 seconds, 45° C. for 60 seconds) × 10 |
| | cycles, 95° C. for 1 minute, (Series 2 -- 95° C. for 5 seconds, 55° C. for 30 seconds) × 50 |
| | cycles |
| 12 | 94° C. for 10 seconds, (Series 1F -- 94° C. for 30 seconds, 45° C. for 60 seconds) × 10 |
| | cycles, 95° C. for 1 minute, (Series 2 -- 95° C. for 5 seconds, 55° C. for 30 seconds) × 50 |
| | cycles |

Results of the experiments are tabulated below in Table 11. Ct values ranged from 14.53 to 27.28, with reaction mixtures 2-5 having no detected product. Generally speaking, reaction mixtures not subjected to multiple series of amplification reactions had either no detectable product or had higher Ct values than reaction mixtures subjected to multiple series of amplification reaction. Results indicate that protocols comprising multiple series of amplification reactions can be useful in achieving good sensitivity. Moreover, results also indicate that protocols comprising multiple series of amplification reactions may achieve better sensitivity when compared to protocols with only a single series. In some cases, multiple series of amplification reactions may be necessary for producing detectable quantities of amplified product.

TABLE 11
Experimental Results of Example 8

| Reaction | |
|---|---|
| Mixture | Ct |
| 1 | 26.03 |
| 2 | — |
| 3 | — |
| 4 | — |
| 5 | — |
| 6 | 27.28 |
| 7 | 21.64 |
| 8 | 19.56 |
| 9 | 17.2 |
| 10 | 14.53 |
| 11 | 19.2 |
| 12 | — |

### Example 9: Comparing Results with Purified and Unpurified Sample

Amplification and detection experiments were performed to compare results obtained with purified and unpurified samples. Purified and un-purified biological samples comprising H1N1 were subject an amplification protocol. Each biological sample was obtained directly from a subject via an oropharyngeal swab. One microliter of each sample was combined in a 25 μL reaction tube with reagents necessary to conduct nucleic acid amplification and to detect amplified product as described herein to obtain a reaction mixture. Three reaction mixtures were generated, with two of the reaction mixtures comprising sample purified by one of column purification or magnetic purification. The third reaction mixture comprised unpurified sample.

The reaction mixtures were incubated according to an amplification protocol comprising 2 minutes at 94° C., 20 minutes at 45° C., 1 minute at 94° C., followed by 50 cycles of 5 seconds at 94° C. and 35 seconds at 55° C. in a real time PCR thermocycler. Detection of amplified product occurred during incubations.

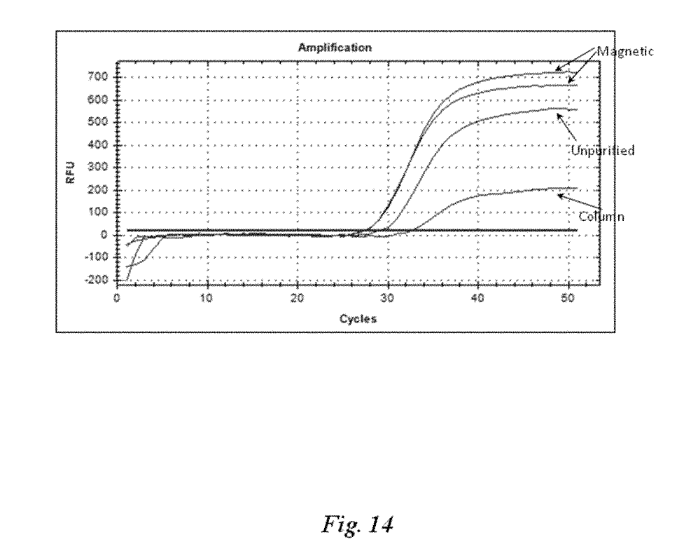

Results of the experiments are graphically depicted in FIG. 14 and shown below in Table 12. As shown in Table 12, determined Ct values ranged from 27 to 31 and were similar between unpurified sample and sample purified by various approaches. Results indicate that purification of sample may be not necessary to achieve similar detection sensitivity.

TABLE 12
Experimental Results of Example 9

| Sample Type | Ct |
|---|---|
| Column Purification | 31 |
| Magnetic Beads Purification | 27 |
| Unpurified | 28 |

### Example 10: Analysis of Whole Blood and Saliva Samples

Amplification and detection experiments were performed on H3N2 virus-containing blood and saliva samples. Four different samples were tested. Two samples comprising either of the whole blood or saliva samples and two samples comprising a 10-fold dilution (in PBS) of either of the whole blood or saliva samples. Each of the four samples was combined with reagents necessary to conduct reverse transcription of the viral RNA and reagents necessary to complete amplification of the complementary DNA obtained from reverse transcription. The reagents necessary to conduct reverse transcription and DNA amplification were supplied as a commercially available pre-mixture (e.g., Takara One-Step RT-PCR or One-Step RT-qPCR kit) comprising reverse transcriptases (e.g., Sensiscript and Omniscript transcriptases), a DNA Polymerase (e.g., HotStarTaq DNA Polymerase), and dNTPs. Moreover, the reaction tubes also included a TaqMan probe comprising a FAM dye for detection of amplified DNA product. To generate amplified DNA product, each reaction mixture was incubated according to a protocol of denaturing and elongation conditions comprising 20 minutes at 45° C., followed by 2 minutes at 94° C., followed by 42 cycles of 5 seconds at 94° C. and 35 seconds at 55° C. in a real-time PCR thermocycler. Detection of amplified product occurred during incubations.

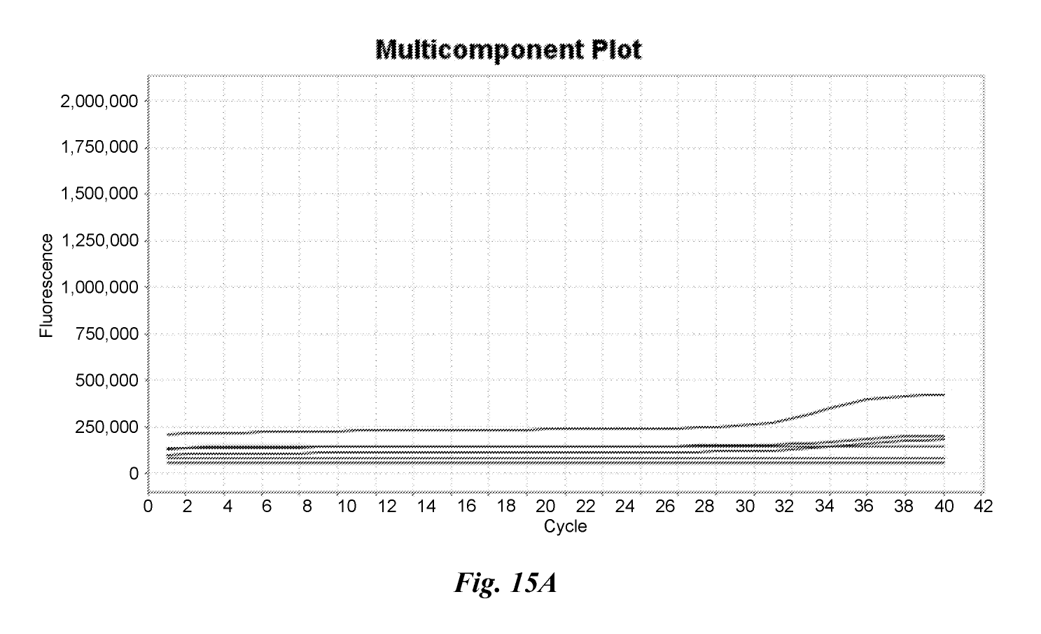

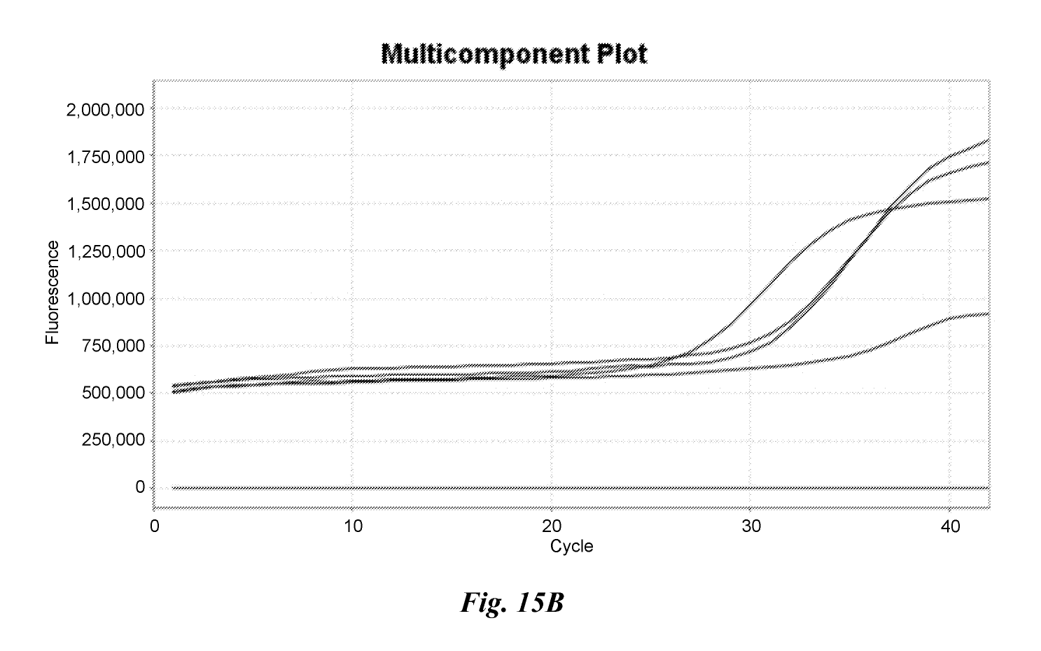

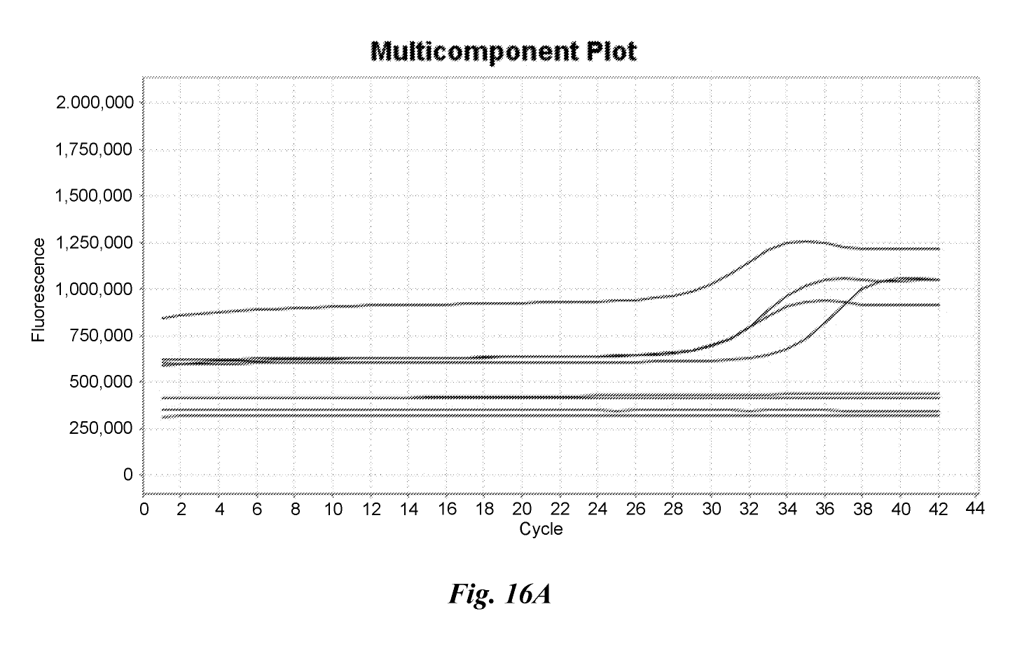

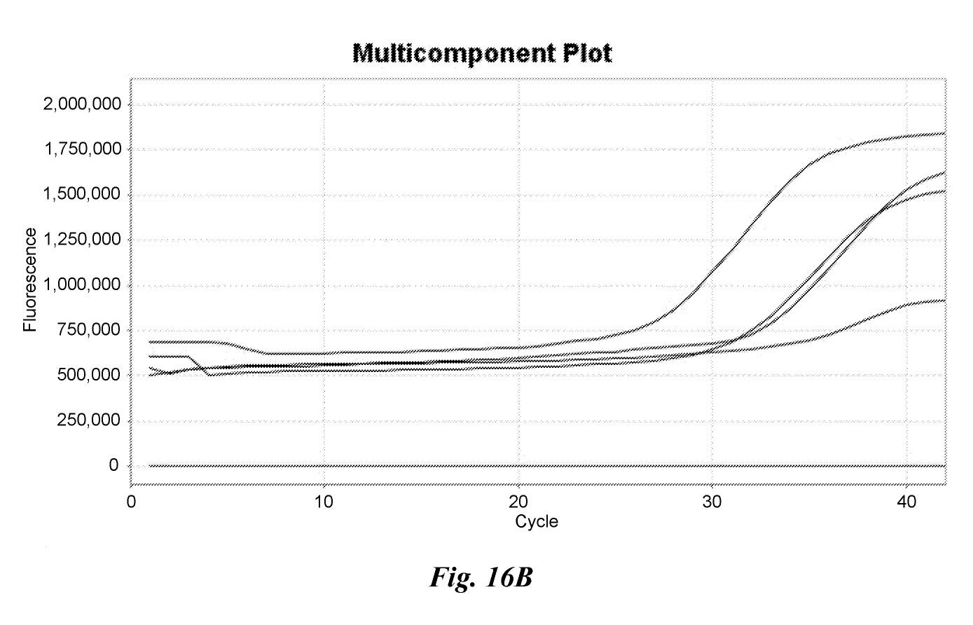

Amplification results for H3N2 are graphically depicted in FIG. 15 (FIG. 15A corresponds to non-diluted blood, FIG. 15B corresponds to diluted blood) and FIG. 16 (FIG. 16A corresponds to non-diluted saliva, FIG. 16B corresponds to diluted saliva). Recorded fluorescence of the FAM dye is plotted against the number of cycles.

As shown in FIG. 15 and FIG. 16, both non-diluted and diluted blood and saliva reaction mixtures showed detectable signal, with Ct values ranging from 24-33. Thus, the data shown in FIGS. 15 and 16 indicate that non-dilute biological samples may be analyzed with good sensitivity, with Ct values of no more than about 40. Moreover, data also indicate that, in cases where dilution of sample is necessary for analysis, amplified product may also be detected in a similar fashion. In some cases, if the inhibitors from samples are too much, dilution may be another way to eliminate the inhibition from the sample, for example, whole blood.

### Example 11: Nested PCR

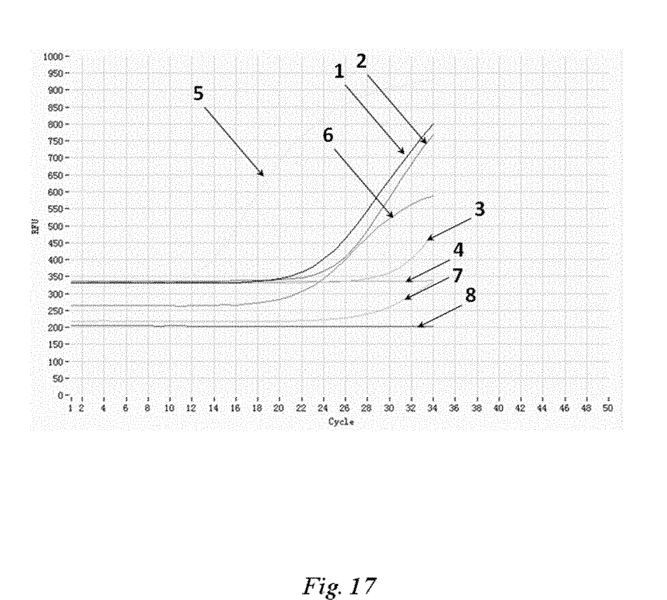

Amplification and detection experiments are performed on H1N1 virus-containing samples. Eight samples are tested. The samples each include H1N1 (2007) virus stock. The samples are each diluted in PBS, at dilutions indicated in Table 13 below. The concentration of virus stock is 1×10⁶ IU/mL. To generate amplified DNA product, a reaction mixture comprising a given sample is incubated according to a protocol of denaturing and elongation conditions. The protocol comprises: (i) in a first run, heating the mixture in a thermocycler at 94° C. for 1 minute followed by 10 or 15 cycles (as indicated in Table 13 below) of 5 seconds at 94° C. and 10 seconds at 57° C.; and (ii) in a second run, heating the mixture in the thermocycler at 94° C. for 1 minute followed by 35 cycles of 5 seconds at 94° C. and 30 seconds at 57° C. A 1 μL series dilution sample is added to a Takara One-step qPCR pre-mixture in a 25 uL reaction volume. After the first run for certain cycles, 1 μL from the reaction is added to the second run reaction mixture. Amplification results for H1N1 are graphically depicted in FIG. 17. The figure shows recorded relative fluorescence units (RFU) as a function of cycle number. Plots for each of the eight samples (1-8) have been indicated in the figure. Samples with detectable signals have Ct values indicated in Table 13.

TABLE 13
Experimental Results of Example 11

| # | 1 | 2 | 3 | 4 | 5 | 6 | 7 | 8 |
|---|---|---|---|---|---|---|---|---|
| Sample | 1/10 | 1/100 | 1/1000 | 0 | 1/10 | 1/100 | 1/1000 | 0 |
| dilution | | | | | | | | |
| Ct | 18 | 21 | 27 | — | 11 | 17 | 24 | — |

| | | |
|---|---|---|
| Cycles | 10 cycles | 15 Cycles |
| of the | | |
| first run | | |

### Example 12: Amplification and Detection of Ebola Recombinant Plasmid

Amplification and detection experiments were performed on human whole blood samples comprising various copy numbers of recombinant plasmid corresponding to the Zaire Ebola Virus (Zaire-EBOV). Eight samples were tested. Six of the samples included the recombinant plasmid at a particular copy number (250000, 25000, 2500, 250, 25 and 2.5 copies) and two of the samples (one having blood only, one having water only) served as control samples. Whole blood samples were analyzed without sample purification.

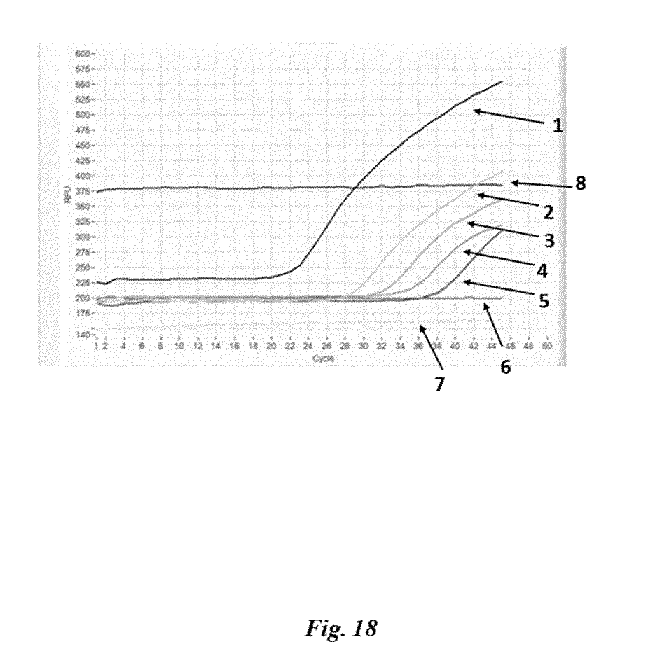

Each sample was combined with reagents necessary for nucleic acid amplification (e.g., DNA polymerase, dNTPs, primers, co-factors, suitable buffer, primers, etc.) and a reporter agent (e.g., an oligonucleotide probe comprising FAM dye) into a reaction mixture. A summary of the various reaction mixtures by sample number, including copy number of recombinant plasmid, is shown in Table 14. To generate amplified product, each reaction mixture was subjected to two series of denaturing and elongation conditions. The two series were as follows: (i) in a first series, 15 cycles of 1 second at 95° C. and 1 second at 45° C., followed by 1 min at 95° C.; and (ii) in a second series, 45 cycles of 5 seconds at 95° C. and 10 seconds at 55° C. During the second series, signal from the reporter agent was recorded to generate amplification curves and obtain Ct values. Amplification curves for the experiments are graphically depicted in FIG. 18, each labeled by sample number corresponding to sample numbers shown in Table 14. Results depicted in FIG. 18 show recorded relative fluorescence units (RFU) as a function of cycle number. Ct values obtained from the curves shown in FIG. 18 are summarized in Table 15.

As shown in FIG. 18, recombinant plasmid was detected via amplified products for all of the samples that included recombinant plasmid except for sample 6. Moreover, recombinant plasmid was not detected in either of the control samples (samples 7 and 8). Accordingly, results shown in FIG. 18 indicate that, in some cases, a detection sensitivity of 25 copies of plasmid/rxn can be obtained using multiple series of denaturing and elongation conditions and without sample purification.

TABLE 14
Experimental reaction mixtures of Example 12

| Sample | plasmid (copies/rxn) |
|---|---|
| 1 | 250000 |
| 2 | 25000 |
| 3 | 2500 |
| 4 | 250 |
| 5 | 25 |
| 6 | 2.5 |
| 7 | 0 (blood only) |
| 8 | 0 (water only) |

TABLE 15
Determined Ct values from experiments in Example 12

| Sample | Copies/rxn | Ct |
|---|---|---|
| 1 | 250000 | 26.12 |
| 2 | 25000 | 33.61 |
| 3 | 2500 | 37.61 |
| 4 | 250 | 40.61 |
| 5 | 25 | 42.97 |
| 6 | 2.5 | — |
| 7 | 0 | — |
| 8 | 0 | — |

### Example 13: Amplification and Detection of Ebola Virus

Amplification and detection experiments were performed on human whole blood samples comprising various copy numbers of Zaire Ebola Virus (Zaire-EBOV) pseudovirus. Eight samples were tested in duplicate (duplicate set #1 and duplicate set #2) for a total of sixteen samples. Six of the samples included the pseudovirus at a particular copy number (2500000, 250000, 25000, 2500, 250 and 25 copies) and two of the samples (one having blood only, one having water only) served as control samples. Whole blood samples were analyzed without sample purification.

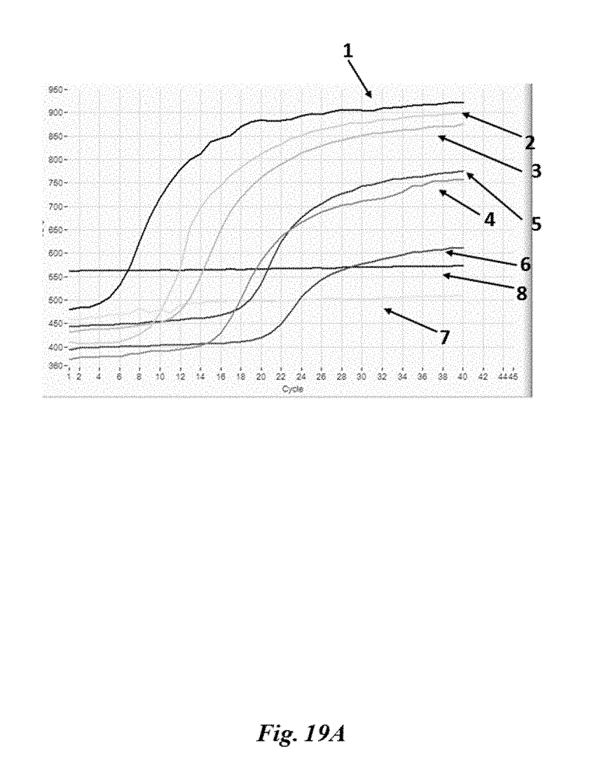

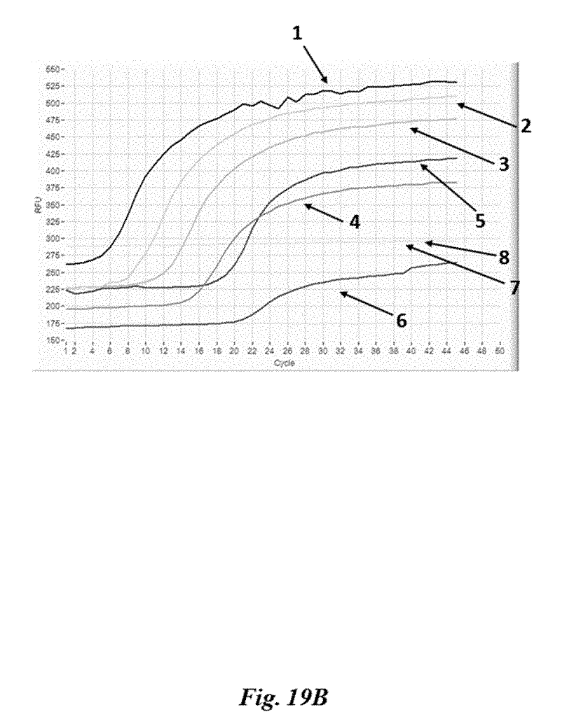

Each sample was combined with reagents necessary for reverse transcription and nucleic acid amplification (e.g., reverse transcriptase, DNA polymerase, dNTPs, co-factors, primers, suitable buffer, etc.) and a reporter agent (e.g., an oligonucleotide probe comprising FAM dye) into a 30 μL reaction mixture. A summary of the reaction mixtures by sample number, including copy number of pseudovirus, is shown in Table 16. To generate amplified product from the pseudovirus, each reaction mixture was subjected to two series of denaturing and elongation conditions. The two series were as follows: (i) in a first series, 15 cycles of 1 second at 95° C. and 1 second at 45° C., followed by 1 min at 95° C.; and (ii) in a second series, 45 cycles of 5 seconds at 95° C. and 10 seconds at 55° C. During the second series, signal from the reporter agent was recorded to generate amplification curves and obtain Ct values Amplification curves for the experiments are graphically depicted in FIG. 19A (duplicate set #1) and FIG. 19B (duplicate set #2), each labeled by sample number corresponding to those shown in Table 16. Results depicted in FIG. 19A and FIG. 19B show recorded relative fluorescence units (RFU) as a function of cycle number. Ct values obtained from the curves shown in FIG. 19A and FIG. 19B are summarized in Table 17, with “Ct 1” corresponding to duplicate set #1 and “Ct 2” corresponding to duplicate set #2.

As shown in FIG. 19A and FIG. 19B, pseudovirus was detected, in both duplicate sets, via amplified products for all of the samples that included pseudovirus (samples 1-6). Moreover, pseudovirus was not detected in any of the control samples (samples 7 and 8). Accordingly, results shown in FIG. 19A and FIG. 19B indicate that, in some cases, a detection sensitivity of 25 copies of virus/rxn can be obtained using multiple series of denaturing and elongation conditions without sample purification.

TABLE 16
Experimental reaction mixtures of Example 13

| Sample | Pseudovirus (copies/rxn) |
|---|---|
| 1 | 2500000 |
| 2 | 250000 |
| 3 | 25000 |
| 4 | 2500 |
| 5 | 250 |
| 6 | 25 |
| 7 | 0 (blood only) |
| 8 | 0 (water only) |

TABLE 17
Determined Ct values from experiments in Example 13

| Sample | Copies/rxn | Ct 1 | Ct 2 |
|---|---|---|---|
| 1 | 2500000 | 8.57 | 8.44 |
| 2 | 250000 | 12.09 | 11.27 |
| 3 | 25000 | 15.03 | 14.99 |
| 4 | 2500 | 18.90 | 18.87 |
| 5 | 250 | 21.71 | 21.71 |
| 6 | 25 | 27.86 | 39.42 |
| 7 | 0 (blood only) | — | — |
| 8 | 0 (water only) | — | — |

### Example 14: Amplification and Detection of Ebola Virus

Amplification and detection experiments were performed on human whole blood samples comprising various copy numbers of Zaire Ebola Virus (Zaire-EBOV) pseudovirus. Eight samples were tested. Six of the samples included the pseudovirus at a particular copy number (2500000, 250000, 25000, 2500, 250 and 25) and two of the samples (one having 20000 copies of a pseudovirus positive control, one having water only) served as control samples. Whole blood samples were analyzed without sample purification.

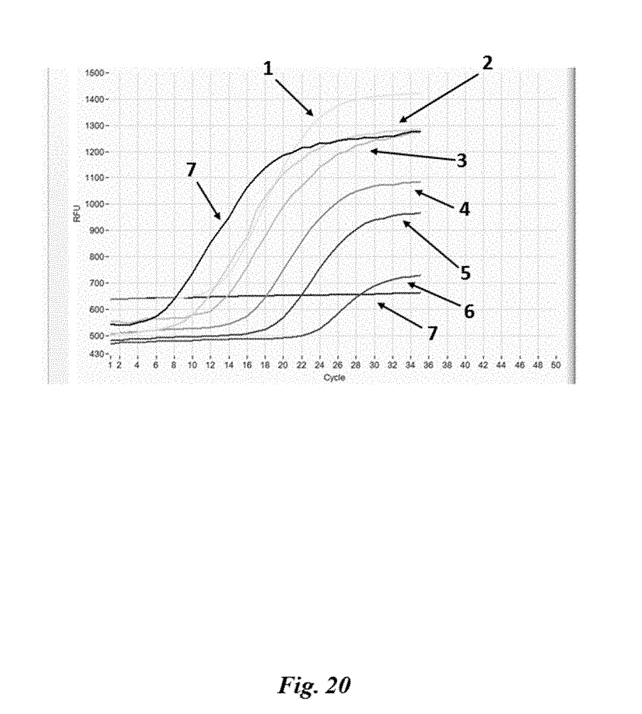

Each sample was combined with reagents necessary for reverse transcription and nucleic acid amplification (e.g., reverse transcriptase, DNA polymerase, primers, dNTPs, co-factors, suitable buffer, etc.) and a reporter agent (e.g., an oligonucleotide probe comprising FAM dye) into a 30 μL reaction mixture. A summary of the various reaction mixtures by sample number, including pseudovirus copy number, is shown in Table 18. To generate amplified product from the pseudovirus, each reaction mixture was subjected to two series of denaturing and elongation conditions. The two series were as follows: (i) in a first series, 15 cycles of 1 second at 95° C. and 1 second at 45° C., followed by 1 min at 95° C.; and (ii) in a second series, 35 cycles of 5 seconds at 95° C. and 10 seconds at 55° C. During the second series, signal from the reporter agent was recorded to generate amplification curves and obtain Ct values Amplification curves for the experiments are graphically depicted in FIG. 20, each labeled by sample number corresponding to those shown in Table 18. Results depicted in FIG. 20 show recorded relative fluorescence units (RFU) as a function of cycle number. Ct values obtained from the curves shown in FIG. 20 are summarized in Table 19.

As shown in FIG. 20, pseudovirus was detected via amplified products for all of the samples that included pseudovirus (samples 1-6), including the sample including positive control pseudovirus (sample 7). Moreover, pseudovirus was not detected in the water only control sample (sample 8). Accordingly, results shown in FIG. 20 indicate that, in some cases, a detection sensitivity of 25 copies of virus/rxn can be obtained using multiple series of denaturing and elongation conditions without sample purification.

TABLE 18
Experimental reaction mixtures of Example 14

| Sample | Pseudovirus (copies/rxn) |
|---|---|
| 1 | 2500000 |
| 2 | 250000 |
| 3 | 25000 |
| 4 | 2500 |
| 5 | 250 |
| 6 | 25 |
| 7 | 20000 (positive control pseudovirus) |
| 8 | 0 (water only) |

TABLE 19
Determined Ct values from experiments in Example 14

| Sample | Copies/rxn | Ct |
|---|---|---|
| 1 | 2500000 | 10.44 |
| 2 | 250000 | 13.30 |
| 3 | 25000 | 16.14 |
| 4 | 2500 | 19.62 |
| 5 | 250 | 22.92 |
| 6 | 25 | 30.00 |
| 7 | 20000 (positive control) | 15.94 |
| 8 | 0 (water only) | — |

### Example 15: Amplification and Detection of Ebola Virus

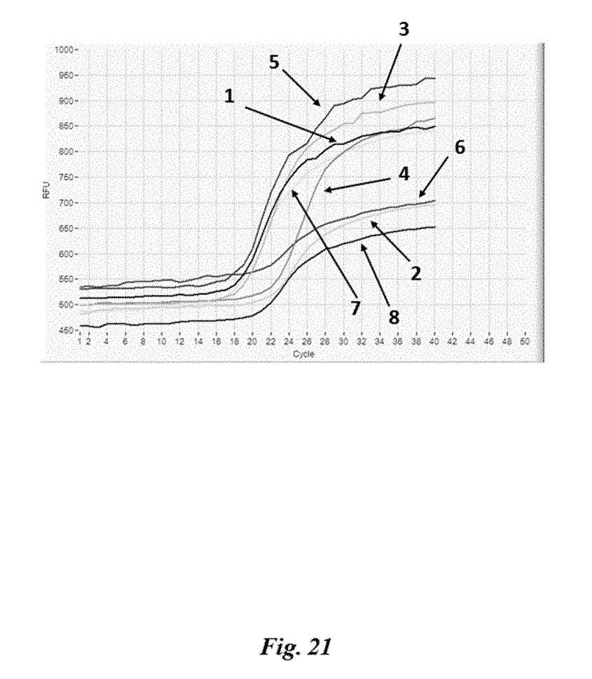

Amplification and detection experiments were performed on human whole blood samples comprising one of two copy numbers (250 copies/rxn or 25 copies/rxn) of Zaire Ebola Virus (Zaire-EBOV) pseudovirus. Each whole blood sample was tested using one of four reagent systems, for a total of eight samples. Each of the reagent systems (B-1, B-2, B-3 and B-4) included reagents necessary for reverse transcription and nucleic acid amplification (e.g., reverse transcriptase, DNA polymerase, primers, dNTPs, co-factors, suitable buffer, etc.) and a reporter agent (e.g., an oligonucleotide probe comprising FAM dye). Each of the different reagent systems contained different concentrations of the various components in the reagent systems. Each whole blood sample was combined with its appropriate reagent system into a 30 μL reaction mixture. A summary of the various reaction mixtures by sample number, including copy number of pseudovirus and reagent system, is shown below in Table 20. To generate amplified product from the pseudovirus, each reaction mixture was subjected to two series of denaturing and elongation conditions. The two series were as follows: (i) in a first series, 15 cycles of 1 second at 95° C. and 1 second at 45° C., followed by 1 min at 95° C.; and (ii) in a second series, 40 cycles of 5 seconds at 95° C. and 10 seconds at 55° C. During the second series, signal from the reporter agent was recorded to generate amplification curves and obtain Ct values Amplification curves for the experiments are graphically depicted in FIG. 21, each labeled by sample number corresponding to those shown in Table 20. Results depicted in FIG. 21 show recorded relative fluorescence units (RFU) as a function of cycle number. Ct values obtained from the curves shown in FIG. 21 are summarized in Table 21.

As shown in FIG. 21, pseudovirus was detected via amplified products for all of the samples, including samples having 25 copies/rxn. Accordingly, results shown in FIG. 21 indicate that, in some cases, a detection sensitivity of 25 copies of virus/rxn can be obtained using multiple series of denaturing and elongation conditions, with different reagent systems and without sample purification.

TABLE 20
Experimental reaction mixtures of Example 15

| Sample | Pseudovirus (copies/rxn) | Reagent System |
|---|---|---|
| 1 | 250 | B-1 |
| 2 | 25 | B-1 |
| 3 | 250 | B-2 |
| 4 | 25 | B-2 |
| 5 | 250 | B-3 |
| 6 | 25 | B-3 |
| 7 | 250 | B-4 |
| 8 | 25 | B-4 |

TABLE 21
Determined Ct values from experiments in Example 15

| Sample | Copies/rxn | Ct |
|---|---|---|
| 1 | 250 | 20.38 |
| 2 | 25 | 24.82 |
| 3 | 250 | 20.62 |
| 4 | 25 | 24.05 |
| 5 | 250 | 20.26 |
| 6 | 25 | 25.09 |
| 7 | 250 | 19.86 |
| 8 | 25 | 24.00 |

### Example 16: Real-Time PCR Detection for Zaire Ebola Virus

A one-step qPCR method of the present disclosure was used to analyze patient blood serum samples for the Zaire Ebola virus. The samples were not purified. The samples included nine Zaire Ebola virus positive samples and seven Zaire Ebola virus negative samples. A Roche LC96 real-time PCR system was used.

The program employed in this example to analyze the samples is shown in Table 22.

TABLE 22
Thermal cycling program

| Step | Tem | Time | | Cycle NO. | |
|---|---|---|---|---|---|
| 1 | 42° C. | 1 | min | 1 | cycle |
| 2 | 95° C. | 5 | second | 10 | cycles |
| | 45° C. | 10 | second | | |
| 3 | 95° C. | 1 | min | 1 | cycle |
| 4 | 95° C. | 5 | second | 40 | cycles |

| | |
|---|---|
| 55° C. | 10 second |
| | (Reading) |

The results of the one-step qPCR method are shown in Table 23. The one-step qPCR method testing for the Zaire Ebola virus showed 100% consistency as compared to a verified reagent and method.

TABLE 23
Results

| | One-Step QPCR | Verified reagent and | |
|---|---|---|---|
| Sample # | Method (Cq) | method (Cq) | consistency |
| 1 | N/A | N/A | Yes |
| 2 | 26.53 | 29.73 | Yes |
| 3 | 17.68 | 19.53 | Yes |
| 4 | N/A | N/A | Yes |
| 5 | N/A | N/A | Yes |
| 6 | N/A | N/A | Yes |
| 7 | N/A | N/A | Yes |
| 8 | 21.52 | 20.98 | Yes |
| 9 | 18.97 | 18.88 | Yes |
| 10 | 24.97 | 24.44 | Yes |
| 11 | 18.92 | 18.91 | Yes |
| 12 | 26.32 | 25.22 | Yes |
| 13 | 20.48 | 20.85 | Yes |
| 14 | 18.5 | 20.45 | Yes |
| 15 | N/A | N/A | Yes |

### Example 17: Amplification and Detection of Malaria

Amplification and detection experiments were performed on a human whole blood sample comprising an unknown concentration of Malaria pathogens. Two sets of experiments were completed. In the first set of experiments, duplicate experiments were completed for a 1:4 dilution (in 1×PBS) of the human whole blood sample; an experiment was completed for a sample comprising whole blood and a plasmid corresponding to Malaria pathogens; and an experiment was completed for a water only control. In the second set of experiments, experiments were completed for samples comprising various dilutions (1:4, 1:40, 1:400, 1:4000, 1:40000 and 1:400000) of the human whole blood sample in 1×PBS along with blood only and water only control samples. Whole blood samples were analyzed without sample purification.

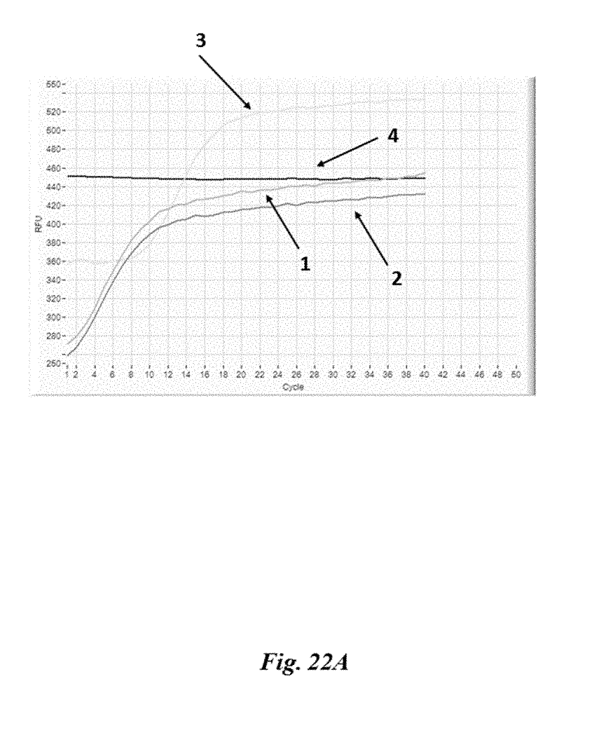

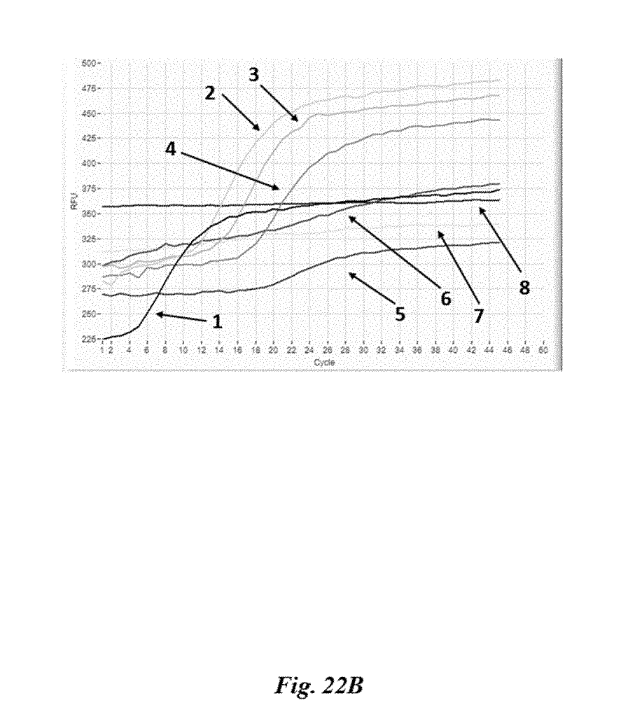

Each sample was combined with reagents necessary for nucleic acid amplification (e.g., DNA polymerase, primers, dNTPs, co-factors, suitable buffer, etc.) and a reporter agent (e.g., an oligonucleotide probe comprising FAM dye) into a 30 μL reaction mixture. A summary of the reaction mixtures by sample number, including dilution, for the first set of experiments is shown in Table 24. A summary of the reaction mixtures by sample number, including dilution, for the second set of experiments is shown in Table 25. To generate amplified product from Malaria pathogens, each reaction mixture was subjected to two series of denaturing and elongation conditions. The two series were as follows: (i) in a first series, 13 cycles of 1 second at 95° C. and 1 second at 45° C., followed by 1 min at 95° C.; and (ii) in a second series, 45 cycles of 5 seconds at 95° C. and 10 seconds at 55° C. During the second series, signal from the reporter agent was recorded to generate amplification curves. Amplification curves for the first set of experiments are graphically depicted in FIG. 22A and amplification curves for the second set of experiments are graphically depicted in FIG. 22B. Each curve is labeled by its corresponding sample number in Tables 24 and 25, respectively. Results depicted in FIG. 22A and FIG. 22B show recorded relative fluorescence units (RFU) as a function of cycle number.

As shown in FIG. 22A, Malaria pathogens were detected via amplified products for the two reaction mixtures comprising whole blood sample (samples 1 and 2) and for the positive control comprising recombinant plasmid (sample 3). Moreover, Malaria pathogens were not detected in the water only control sample (sample 4). Accordingly, results shown in FIG. 22A indicate that Malaria pathogens can, in some cases, be detected using multiple series of elongation and denaturation conditions without sample purification.

As shown in FIG. 22B, Malaria pathogens were detected via amplified products for all reaction mixtures containing whole blood sample (samples 1-6). Moreover, Malaria pathogens were not detected in the water only and blood only control samples (sample 7 and 8). Accordingly, results shown in FIG. 22B indicate that a pathogen(s), including Malaria pathogens, can, in some cases, be detected at dilutions of up to 1:400000 using multiple series of denaturing and elongation conditions and without sample purification.

TABLE 24
Experimental reaction mixtures for first
set of experiments in Example 17

| Sample | Dilution |
|---|---|
| 1 | 1:4 |
| 2 | 1:4 |
| 3 | 1:2 (plasmid in whole |
| | blood control) |
| 4 | None (water only) |

TABLE 25
Experimental reaction mixtures for second
set of experiments in Example 17

| Sample | Dilution |
|---|---|
| 1 | 1:4 |
| 2 | 1:40 |
| 3 | 1:400 |
| 4 | 1:4000 |
| 5 | 1:40000 |
| 6 | 1:400000 |
| 7 | 0 (blood only) |
| 8 | 0 (water only) |

### Example 18: Amplification and Detection of Dengue Virus

Amplification and detection experiments were performed on samples obtained from a culture comprising an unknown concentration of Dengue virus. Three sets of experiments were completed. In the first set of experiments, duplicate experiments were completed for undiluted culture; an experiment was completed for 1:10 dilution of the culture; and an experiment was completed for a water only control. In the second set of experiments, experiments were completed for various dilutions (no dilution, 1:10, 1:100, 1:1000, 1:10000, 1:100000 and 1:1000000) of the culture along with a water only control sample. In the third set of experiments, experiments were completed for various dilutions (no dilution, 1:10, 1:100, 1:1000 and 1:10000) of the culture along with a water only control sample. Culture samples were analyzed without sample purification.

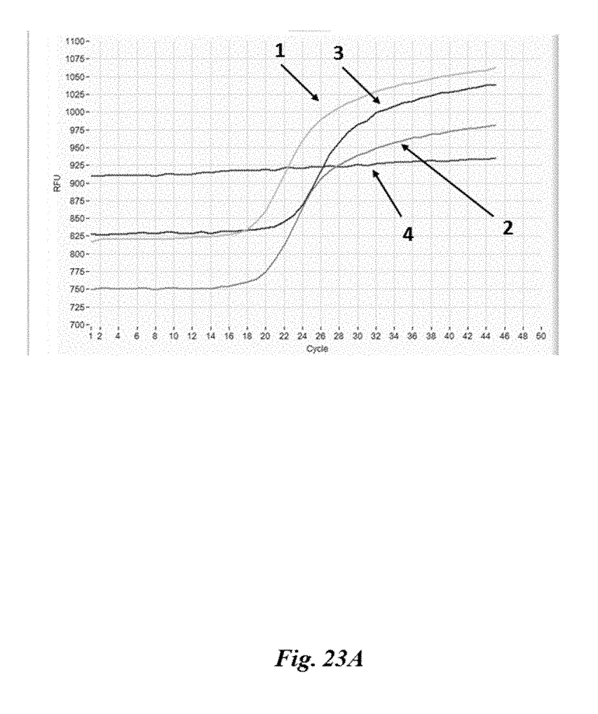

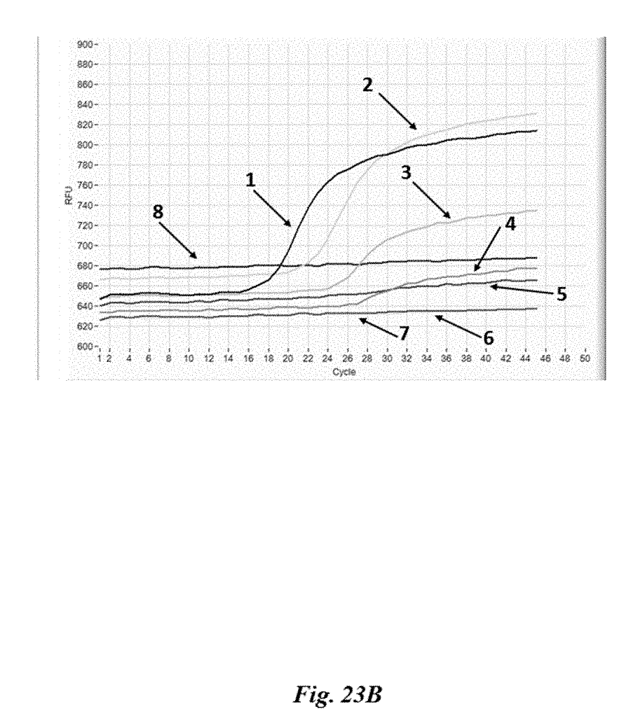

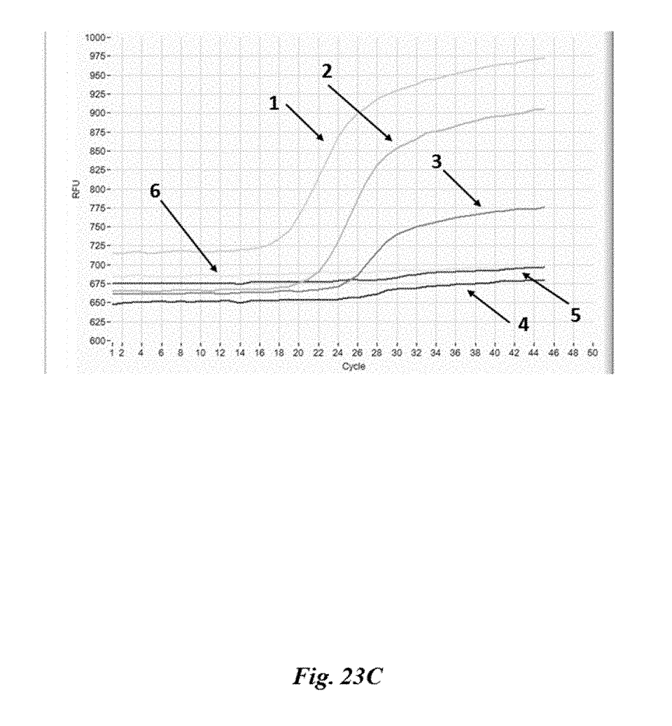

2 μL of each sample was combined with reagents necessary for reverse transcription and nucleic acid amplification (e.g., reverse transcriptase, DNA polymerase, primers, dNTPs, co-factors, suitable buffer, etc.) and a reporter agent (e.g., an oligonucleotide probe comprising FAM dye) into a 30 μL reaction mixture. A summary of the reaction mixtures, including dilution, for the first set of experiments is shown in Table 26, for the second set of experiments in Table 27 and for the third set of experiments in Table 28. To generate amplified product from the virus, each reaction mixture was subjected to two series of denaturing and elongation conditions. The two series were as follows: (i) in a first series, 1 min at 42° C., 10 cycles of 5 seconds at 95° C. and 10 seconds at 45° C., followed by 1 min at 95° C.; and (ii) in a second series, 45 cycles of 5 seconds at 95° C. and 10 seconds at 55° C. During the second series, signal from the reporter agent was recorded to generate amplification curves. Amplification curves for the first set of experiments are graphically depicted in FIG. 23A, amplification curves for the second set of experiments are graphically depicted in FIG. 23B and amplification curves for the third set of experiments are graphically depicted in FIG. 23C. Each curve is labeled by its corresponding sample number in Tables 26, 27 and 28 respectively. Results depicted in FIG. 23A, FIG. 23B and FIG. 23C show recorded relative fluorescence units (RFU) as a function of cycle number. Ct values obtained from the curves shown in FIG. 23A, FIG. 23B and FIG. 23C are shown in Tables 26, 27 and 28 respectively.

As shown in FIG. 23A, virus was detected via amplified products for the three reaction mixtures comprising virus (samples 1-3). Moreover, virus was not detected in the water only control sample (sample 4). Accordingly, results shown in FIG. 23A indicate that Dengue virus can, in some cases, be detected using multiple series of elongation and denaturation conditions.

As shown in FIG. 23B, virus was detected via amplified products for reaction mixtures containing Dengue virus and either not diluted (sample 1) or diluted up to 1:1000 (samples 2, 3 and 4). A Ct value, however, was not determined for the 1:1000 reaction mixture (sample 4). Virus was not detected in higher dilutions (samples 5, 6 and 7) or in the water only control sample (sample 8). Accordingly, results shown in FIG. 23B indicate that virus can, in some cases, be detected at dilutions of up to 1:1000, where Ct values can be generated at dilutions up to 1:100 using multiple series of denaturing and elongation conditions and without sample purification.

As shown in FIG. 23C, virus was detected via amplified products for reaction mixtures containing Dengue virus and either not diluted (sample 1) or diluted up to 1:1000 (samples 2, 3 and 4). A Ct value, however, was not determined for the 1:1000 reaction mixture. Virus was not detected in higher dilutions (sample 5) or in the water only control sample (sample 6). Accordingly, results shown in FIG. 23C indicate that virus can, in some cases, be detected at dilutions of up to 1:1000, where Ct values can be generated at dilutions up to 1:100 using multiple series of denaturing and elongation conditions and without sample purification.

TABLE 26
Experimental reaction mixtures and determined Ct
values for first set of experiments in Example 18

| Sample | Dilution | Ct value |
|---|---|---|
| 1 | none | 19.32 |
| 2 | none | 20.40 |
| 3 | 1:10 | 23.23 |
| 4 | no virus (water only) | — |

TABLE 27
Experimental reaction mixtures and determined Ct values
for second set of experiments in Example 18

| Sample | Dilution | Ct value |
|---|---|---|
| 1 | none | 20.85 |
| 2 | 1:10 | 25.14 |
| 3 | 1:100 | 31.57 |
| 4 | 1:1000 | — |
| 5 | 1:10000 | — |
| 6 | 1:100000 | — |
| 7 | 1:1000000 | — |
| 8 | no virus (water only) | — |

TABLE 28
Experimental reaction mixtures and determined Ct
values for third set of experiments in Example 18

| Sample | Dilution | Ct value |
|---|---|---|
| 1 | None | 19.22 |
| 2 | 1:10 | 22.43 |
| 2 | 1:100 | 26.55 |
| 4 | 1:1000 | — |
| 5 | 1:10000 | — |
| 6 | no virus (water only) | — |

### Example 19: Detection of Single Nucleotide Polymorphisms (SNPs)

Amplification and detection experiments were performed on human oropharyngeal swab or blood samples comprising a particular genotype of cytochrome P450 2C19, CYP2C19*2 (having a “GA” genotype) or CYP2C19*3 (having a “GG” genotype). Two sets of experiments were conducted—one set for samples obtained from human oropharyngeal swabs and one set for samples obtained from blood. In the first set of experiments, seven different samples obtained from human oropharyngeal swabs were analyzed without sample purification. In the second set of experiments, five different blood samples were analyzed without sample purification.

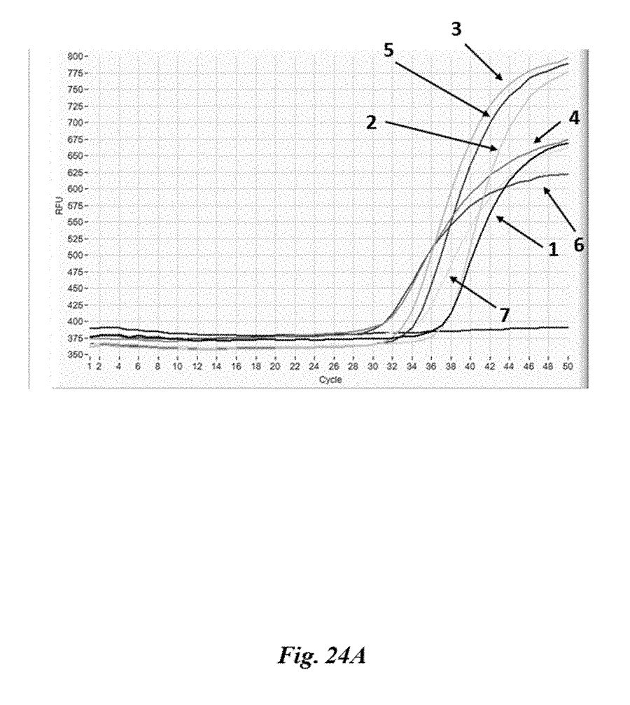

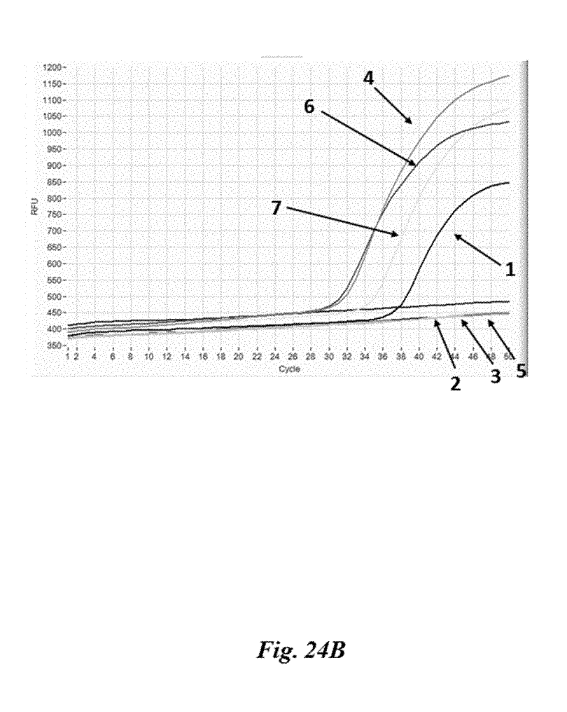

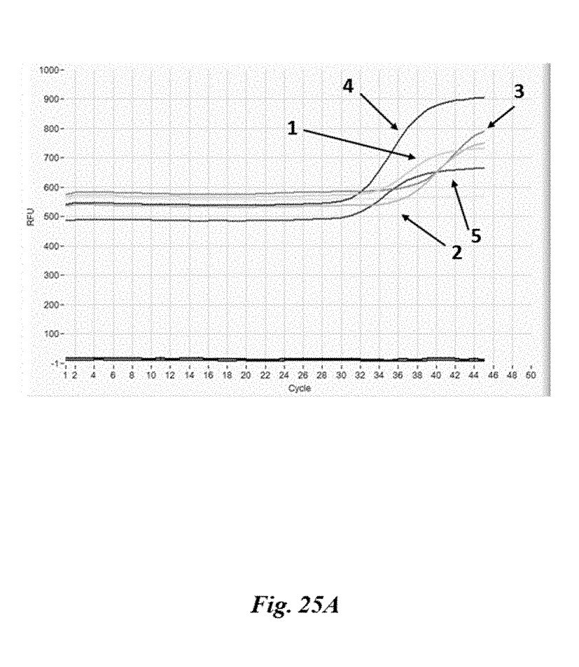

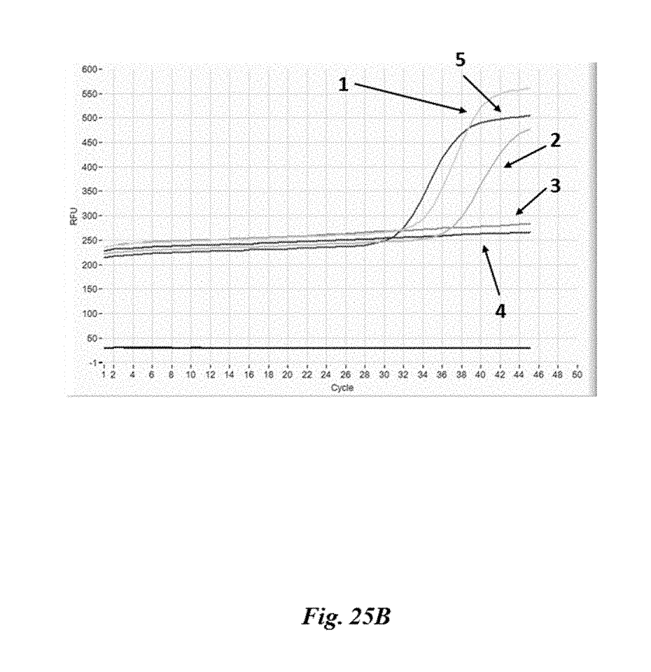

Each sample was combined with reagents necessary for nucleic acid amplification (e.g., DNA polymerase, primers, dNTPs, co-factors, suitable buffer, etc.) and two reporter agents (e.g., an oligonucleotide probe comprising FAM dye to detect amplification of nucleic acids, an oligonucleotide probe comprising Texas Red dye to detect the “GA” genotype) into a reaction mixture. To generate amplified product, each reaction mixture was subjected to a thermocycling protocol that included 5 min at 95° C. followed by 50 cycles of 5 seconds at 95° C. and 10 seconds at 49° C. During thermocycling, signals from the reporter agents were recorded to generate amplification curves. Amplification curves for the first set of experiments (human oropharyngeal swabs) are graphically depicted in FIG. 24A (signal corresponding to the FAM oligonucleotide probe) and FIG. 24B (signal corresponding to the Texas Red oligonucleotide probe). Amplification curves for the second set of experiments (blood samples) are graphically depicted in FIG. 25A (signal corresponding to the FAM oligonucleotide probe) and FIG. 25B (signal corresponding to the Texas Red oligonucleotide probe). Results depicted in FIG. 24A, FIG. 24B, FIG. 25A and FIG. 25B show recorded relative fluorescence units (RFU) as a function of cycle number. Each curve is labeled by its corresponding reaction mixture number in Table 29 (oropharyngeal swab experiments) or Table 30 (blood experiments). Ct values determined for amplification curves are also shown in Table 29 or Table 30 along with determined genotype. In amplification curves where signal from Texas Red was observed in FIG. 24B or FIG. 25B, it was determined that the corresponding reaction mixture had the “GA” genotype. Moreover, in amplification curves where signal from Texas Red was not observed in FIG. 24B or FIG. 25B, it was determined that the corresponding reaction mixture had the “GG” genotype.

As shown in FIG. 24A, amplified product was observed for each of the reaction mixtures having sample obtained from oropharyngeal swabs, suggesting that amplification of nucleic acids occurs. However, as shown in FIG. 24B, amplified product was observed for only some of the reaction mixtures (reaction mixtures 1, 4, 6 and 7) having sample obtained from oropharyngeal swabs, these reaction mixtures corresponding to the “GA” genotype. In the other reaction mixtures (reaction mixtures 2, 3 and 5), amplified products were not observed, these reaction mixtures corresponding to the “GG” genotype. Results shown in FIG. 24A and FIG. 24B were validated via amplification and detection experiments using DNA extracted from oral swab samples (data not shown). Thus, results shown in FIG. 24A and FIG. 24B suggest that, in some cases, SNPs can be detected via real-time amplification in samples obtained from oropharyngeal swabs without sample purification.

As shown in FIG. 25A, amplified product was observed for each of the reaction mixtures having sample obtained from blood, suggesting that amplification of nucleic acids occurs. However, as shown in FIG. 25B, amplified product was observed for only some of the reaction mixtures (reaction mixtures 1, 2 and 5) having sample obtained from blood, these reaction mixtures corresponding to the “GA” genotype. In the other reaction mixtures (reaction mixtures 3 and 4), amplified products were not observed, these reaction mixtures corresponding to the “GG” genotype. Results shown in FIG. 25A and FIG. 25B were validated using nucleic acid sequencing. Thus, results shown in FIG. 25A and FIG. 25B suggest that, in some cases, SNPs can be detected via real-time amplification in samples obtained from blood without sample purification.

TABLE 29
Determined Ct values and genotypes for oropharyngeal
swab experiments in Example 19

| Reaction | Ct- FAM | Ct-Texas Red | |
|---|---|---|---|
| Mixture | Reporter | Reporter | Genotype |
| 1 | 38.70 | 40.25 | GA |
| 2 | 38.28 | — | GG |
| 3 | 34.16 | — | GG |
| 4 | 33.18 | 33.75 | GA |
| 5 | 35.20 | — | GG |
| 6 | 33.08 | 33.59 | GA |
| 7 | 36.45 | 37.01 | GA |

TABLE 30
Determined Ct values and genotypes
for blood experiments in Example 19

| Reaction | Ct- FAM | Ct-Texas Red | |
|---|---|---|---|
| Mixture | Reporter | Reporter | Genotype |
| 1 | 38.36 | 36.24 | GA |
| 2 | 39.97 | 39.67 | GA |
| 3 | 41.25 | — | GG |
| 4 | 33.96 | — | GG |
| 5 | 35.68 | 34.12 | GA |

### Example 20: Amplification and Detection of Adenovirus Type 55 (ADV55) and Adenovirus Type 7 (ADV7)

Amplification and detection experiments were performed on samples obtained from oropharyngeal swabs comprising various copy numbers of adenovirus type 55 (ADV55) or unknown concentrations of adenovirus type 7 (ADV7). Two sets of experiments were completed—one set for samples having ADV55 and one set for experiments having ADV7. In the first set of experiments, six different experiments having samples comprising differing copy numbers (1, 10, 100, 1000, 10000, and 100000 copies) of ADV55 were completed without sample purification along with an experiments for a negative control. In the second set of experiments, eight different experiments having samples comprising unknown copy number of ADV7 were completed without sample purification.

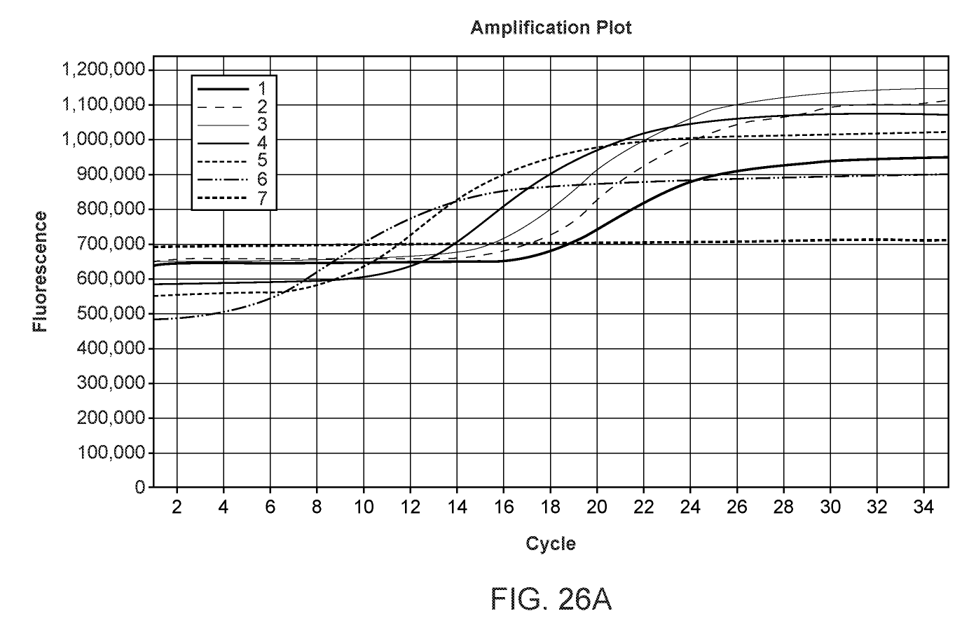

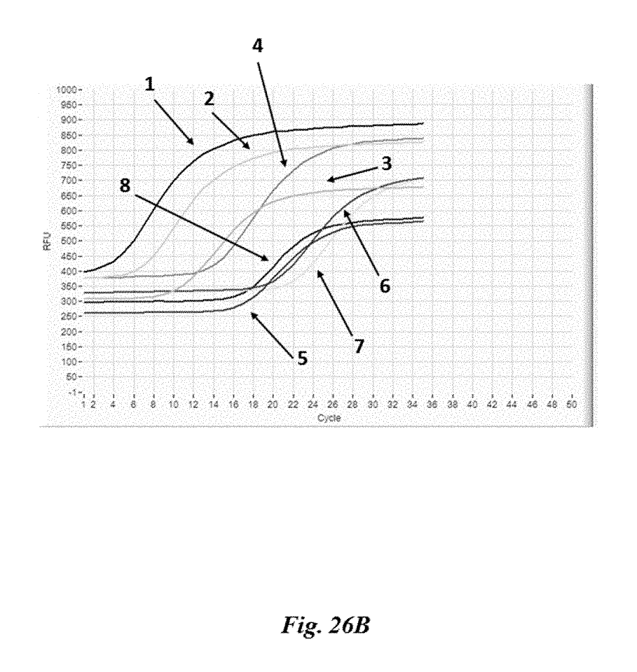

Each sample was combined with reagents necessary for nucleic acid amplification (e.g., DNA polymerase, primers, dNTPs, co-factors, suitable buffer, etc.) and a reporter agent (e.g., an oligonucleotide probe comprising FAM dye) into a reaction mixture. A summary of the reaction mixtures, including ADV55 copy number, for the first set of experiments is shown in Table 31. To generate amplified product from viruses, each reaction mixture was subjected to two series of denaturing and elongation conditions. The two series were as follows: (i) in a first series, 20 cycles of 1 second at 95° C. and 1 second at 45° C., followed by 1 min at 95° C.; and (ii) in a second series, 35 cycles of 5 seconds at 95° C. and 34 seconds at 60° C. During the second series, signal from the reporter agent was recorded to generate amplification curves and obtain Ct values. Amplification curves for the first set of experiments are graphically depicted in FIG. 26A, each labeled by reaction mixture number corresponding to those shown in Table 31. Amplification curves for the second set of experiments are graphically depicted in FIG. 26B and corresponding Ct values shown in Table 32. Amplification curves in FIG. 26B are labelled as they correspond to reaction mixture number shown in Table 32. Results depicted in FIG. 26A and FIG. 26B show recorded relative fluorescence units (RFU) as a function of cycle number.

As shown in FIG. 26A, ADV55 was detected via amplified products for all of the reaction mixtures comprising sample containing virus (reaction mixtures 1-6). Moreover, virus was not detected in the negative control reaction mixture (reaction mixture 7). Accordingly, results shown in FIG. 26A indicate that ADV55 virus can, in some cases, be detected using multiple series of elongation and denaturation conditions without sample purification and at various levels of dilution.

As shown in FIG. 26B, ADV7 was detected via amplified products for all of the reaction mixtures. Accordingly, results shown in FIG. 26B indicate that ADV7 virus can, in some cases, be detected using multiple series of elongation and denaturation conditions and without sample purification.

TABLE 31
Experimental reaction mixtures for
ADV55 experiments in Example 20

| Reaction | ADV55 Copy |
|---|---|
| Mixture | Number/Rxn |
| 1 | 1 |
| 2 | 10 |
| 3 | 100 |
| 4 | 1000 |
| 5 | 10000 |
| 6 | 100000 |
| 7 | 0 (negative |
| | control) |

TABLE 32
Determined Ct values for ADV7 experiments in Example 20

| Reaction | |
|---|---|
| Mixture | Ct Value |
| 1 | 5.12 |
| 2 | 7.16 |
| 3 | 10.97 |
| 4 | 14.15 |
| 5 | 17.58 |
| 6 | 20.29 |
| 7 | 22.13 |
| 8 | 17.66 |

### Example 21: Amplification and Detection of Armored RNA Hepatitis C Virus (RNA-HCV)

Amplification and detection experiments were performed on blood plasma samples comprising various copy numbers of armored RNA Hepatitis C Virus (RNA-HCV). Three different experiments having samples comprising differing copy numbers (10, 100 and 500 copies) of RNA-HCV were completed without sample purification along with an experiment completed for a negative control.

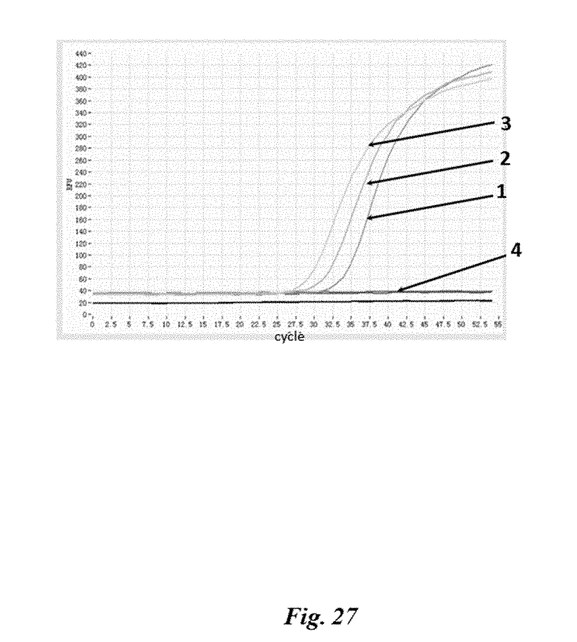

Each sample was combined with reagents necessary for reverse transcription and nucleic acid amplification (e.g., reverse transcriptase, DNA polymerase, primers, dNTPs, co-factors, suitable buffer, etc.) and a reporter agent (e.g., an oligonucleotide probe comprising FAM dye) into a reaction mixture. A summary of the reaction mixtures, including RNA-HCV copy number is shown in Table 33. To generate amplified DNA product from viruses, each reaction mixture was subjected to two series of denaturing and elongation conditions. The two series were as follows: (i) in a first series, 20 cycles of 1 second at 95° C. and 1 second at 45° C., followed by 1 min at 95° C.; and (ii) in a second series, 55 cycles of 5 seconds at 95° C. and 34 seconds at 60° C. During the second series, signal from the reporter agent was recorded to generate amplification curves Amplification curves for the first set of experiments are graphically depicted in FIG. 27, each labeled by number corresponding to reaction mixture numbers shown in Table 33. Results depicted in FIG. 27 show recorded relative fluorescence units (RFU) as a function of cycle number.

As shown in FIG. 27, RNA-HCV was detected via amplified products for all of the reaction mixtures comprising sample containing virus (reaction mixture 1-3). Moreover, RNA-HCV was not detected in the negative control reaction mixture (reaction mixture 4). Accordingly, results shown in FIG. 27 indicate that RNA-HCV can, in some cases, be detected using multiple series of elongation and denaturation conditions without sample purification. A detection sensitivity of 10 copies/rxn can also be achieved.

TABLE 33
Experimental reaction mixtures for
RNA-HCV experiments in Example 21

| Reaction | RNA-HCV Copy |
|---|---|
| Mixture | Number/Rxn |
| 1 | 10 |
| 2 | 100 |
| 3 | 500 |
| 4 | 0 (negative |
| | control) |

### Example 22: Amplification and Detection of Salmonella in Stool Samples

Amplification and detection experiments were performed on stool samples comprising various concentrations of Salmonella. Four different experiments having clinical samples comprising various amounts of Salmonella were completed without sample purification. Among these four clinical samples, only sample No. 4 is Salmonella negative and the other three are Salmonella positive, as determined by PCR analysis using corresponding purified DNA samples from the stool.

For each experiment, 0.2 g stool sample was added into a 1.5 mL centrifuge tube. 200 μl normal saline solution was added into each tube. The tube was then vortexed at maximum speed until the stool sample was thoroughly homogenized.

Then, for each sample, 50 μl of the homogenized stool sample suspension was transferred into a new 1.5 mL centrifuge tube. Then, 50 μl lysis buffer (100 mM NaOH, pH 12.5) was added and mixed thoroughly with the homogenized stool sample. The mixture was then incubated at room temperature for no more than 10 minutes.

Next, the mixture was centrifuged at 12,000 rpm for 1 min. For each sample, 5 μl of the obtained supernatant was then added into a reaction vessel comprising 45 μl amplification reagents for performing PCR.

The amplification reactions were performed according to the protocols described in Table 34 below.

TABLE 34
PCR Protocols Employed in Example 22

| | | | | | |
|---|---|---|---|---|---|
| 1 | 95° C. | 1 | second | 15 | cycles |
| | 50° C. | 1 | second | | |
| 2 | 95° C. | 1 | min | 1 | cycle |
| 3 | 95° C. | 5 | seconds | 35 | cycles |

| | | |
|---|---|---|
| 55° C. | 10 | seconds |

As can be seen from the results shown in Table 35, Salmonella has been successfully detected using the method of the present disclosure without sample purification.

TABLE 35
PCR Amplification Results of Example 22

| Sample | | | |
|---|---|---|---|
| Well | Sample | Channel | Ct value |
| 1 | — | FAM | — |
| 2 | PCR mix NTC | FAM | — |
| 3 | Clinical negative sample NO. 4 | FAM | — |
| 4 | Clinical positive sample NO. 16 | FAM | 21.94 |
| 5 | Clinical positive sample NO. 25 | FAM | 16.37 |
| 6 | Clinical positive sample NO. 43 | FAM | 22.57 |
| 7 | — | FAM | — |
| 8 | — | FAM | — |

Sensitivity of the subject method for detecting Salmonella in stool samples was then determined with the LOD (Limit Of Detection) test. Briefly, the sample was prepared by mixing 45 μl stool suspension not comprising any Salmonella with 5 μl Salmonella dilution of known concentration. The concentration of Salmonella in the sample was then determined by plating 100 μl of the sample on a Luria-Bertani (LB) agar plate, then the total number of viable colonies were counted. The samples were also amplified and analyzed using the method of the present disclosure, as described above. The results showed that the limit of detection (LOD) for Salmonella in stool samples using the method of the present disclosure is 1.1E+3 CFU/mL.

Thus, comparing to other methods and kits commercially available to analyze stool samples (e.g., real time PCR kits provided by Shanghai ZJ Bio-Tech Co., Ltd. or provided by DAAN Gene Co., Ltd.), the method of the present disclosure may not require purification of stool sample, extraction of DNA, or incubation (e.g., for lysis) at elevated temperature (e.g., 100° C. or boiling temperature). The subject method takes less steps and costs much less time, yet, it may be used to successfully detect Salmonella in stool samples with higher sensitivity.

### Example 23: Amplification and Detection of Coxsachie Virus A16 in Stool and Swab Samples

Amplification and detection of Coxsachie Virus A16 in stool samples and throat swab samples was performed. Experiments were conducted with three stool samples and three throat swab samples, along with negative controls.

Throat swab samples were obtained directly from the subjects, and suspended in suspension buffer. Briefly, a fiber tip or a Q-tip was used to swab the posterior pharynx and tonsillar area of a subject, which was then inserted into a sample tube comprising a medium containing a protein stabilizer and a buffer. The sample was mixed thoroughly with the medium. Then, 5-10 μl of each throat swab sample so prepared was added into a reaction vessel for PCR amplification.

As for stool samples, 200 mg stool sample was added into a 2 ml centrifuge tube. Then, 0.2 ml suspension buffer was added to each tube. The samples were vortexed continuously for 1 min or until the stool sample was thoroughly homogenized. In another instance, the homogenized samples were centrifuged at 12000 rpm for 2 min to obtain supernatant comprising the biological samples and pellets. 20 μl of each supernatant obtained was then added into a new 1.5 ml centrifuge tube, and 20 μl lysis buffer was added. The tubes were vortexed continuously at room temperature for 15 s, and centrifuged briefly. 10 μl of each sample was added into a reaction vessel for PCR amplification. The PCR reactions were performed as described in Example 22.

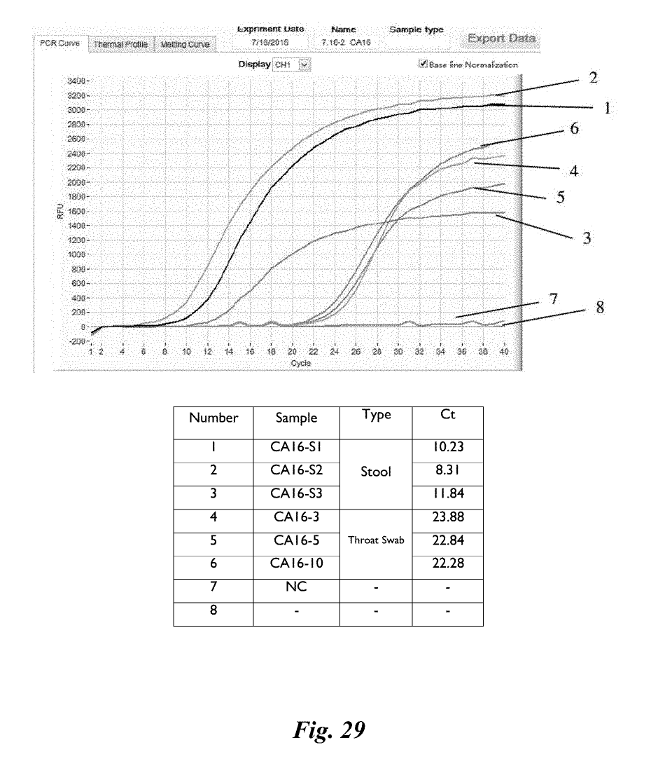

As shown in FIG. 29, Coxsachie Virus A16 in stool and swab samples was successfully detected. The detection sensitivity for Coxsachie Virus A16 was determined to be 25 copies/rnx.

### Example 24: Comparison of Amplification and Detection Results Using Different Methods

Amplification and detection of Coxsachie Virus A16 in throat swab samples was compared using the method as described in Example 23 (i.e., without sample purification and extraction of nucleic acid) and a control method A. In the control method A, viral nucleic acid was purified using a purification kit, wherein the sample was lysed, bound to membranes of a purification column and washed for several times, then the bound nucleic acid was eluted for further analysis.

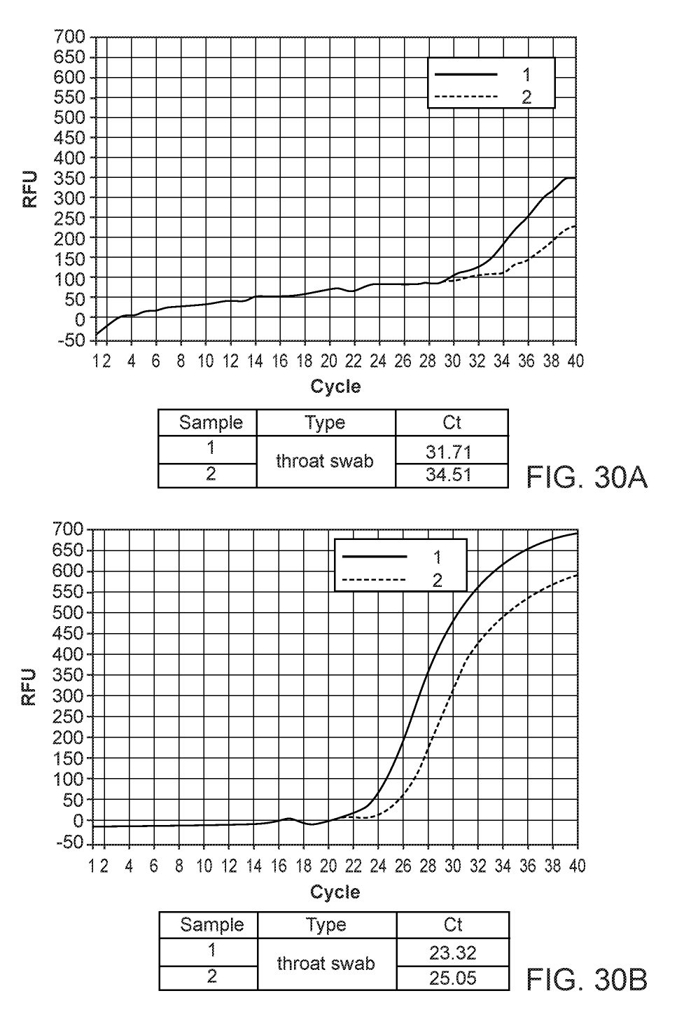

As shown in FIG. 30A and FIG. 30B, the method of the present disclosure enables detection of the Coxsachie Virus A16 with lower Ct values.

### Example 25: Comparison of Amplification and Detection Results Using Different Methods

Amplification and detection of Coxsachie Virus A16 in throat swab samples and stool samples was compared using the method as described in Example 23 (i.e., without sample purification and extraction of nucleic acid) and a control method B. In the control method B, viral nucleic acid was purified using a purification kit, wherein the sample was lysed, bound to membranes of a purification column and washed for several times, then the bound nucleic acid was eluted for further analysis.

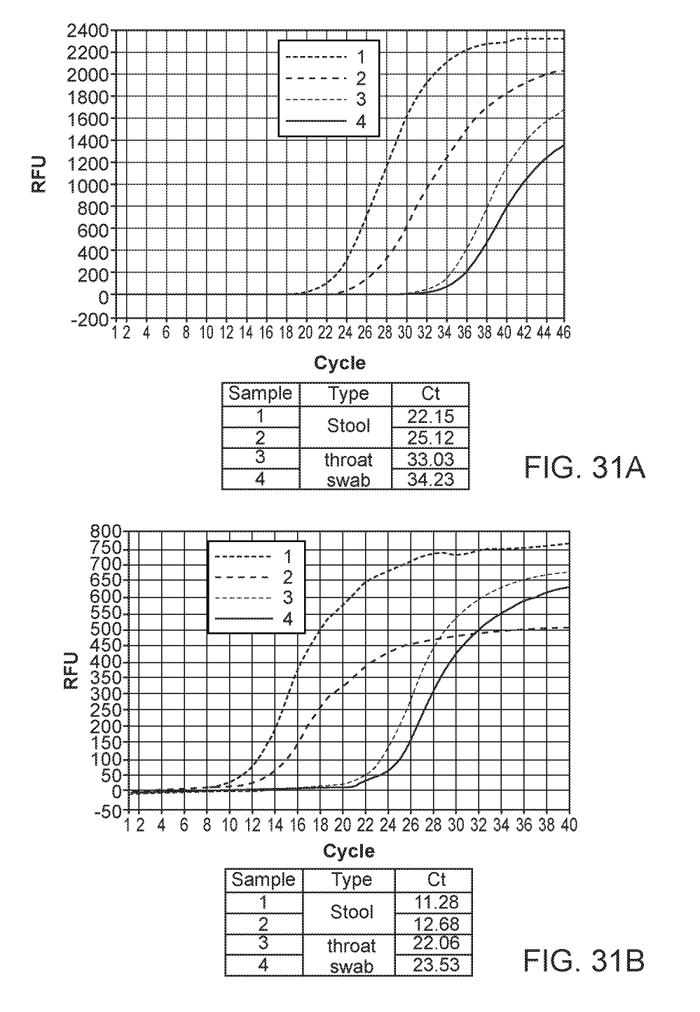

As shown in FIG. 31A and FIG. 31B, the method of the present disclosure enables detection of the Coxsachie Virus A16 with significantly lower Ct values.

### Example 26: Comparison of Amplification and Detection Results Using Different Methods

Amplification and detection of Coxsachie Virus A16 in throat swab samples and stool samples was compared using the method as described in Example 23 (i.e., without sample purification and extraction of nucleic acid) and a control method C. In the control method C, viral nucleic acid was purified using a purification kit, wherein the sample was lysed using Trizol, and treated with chloroform. Then, SiO₂ was used to adsorb the nucleic acid. The samples were then washed and the adsorbed nucleic acids were eluted.

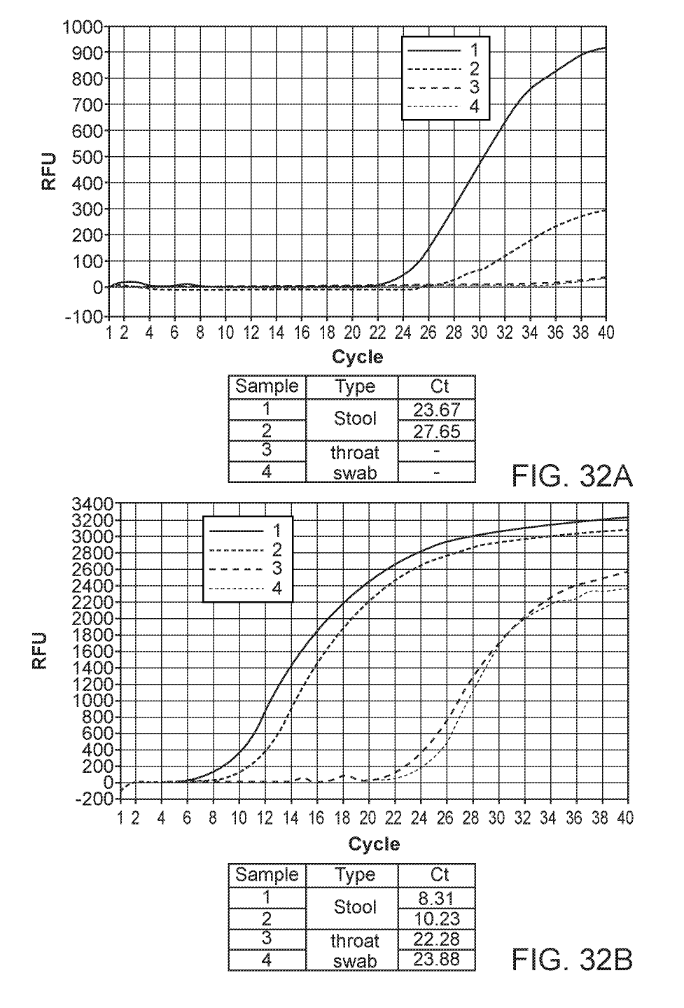

As shown in FIG. 32A and FIG. 32B, the control method C failed to detect Coxsachie Virus A16 in the throat swab samples. For the stool samples, the method of the present disclosure enables detection of the Coxsachie Virus A16 with significantly lower Ct values.

### Example 27: Amplification and Detection of Salmonella in Milk Samples without Enrichment Culturing

Amplification and detection experiments were performed on milk samples comprising unknown concentration of Salmonella, without culture enrichment. A milk sample comprising unknown amount of Salmonella was analyzed without culture enrichment and sample purification. Briefly, 2 mL milk sample was added into a 2 mL centrifuge tube. The tube was then centrifuged at 6,000 rpm for 2 min. The supernatant was removed. Then, 50 μl lysis buffer (100 mM NaOH, pH 12.5) was added and mixed thoroughly. The mixture was then incubated at room temperature for 10 minutes.

Next, 3 μl of the mixture was added into a reaction vessel comprising 47 μl amplification reagents for performing PCR.

The amplification reactions were performed according to the protocols described in Table 36 below.

TABLE 36
PCR Protocols Employed in Example 27

| | | | | | |
|---|---|---|---|---|---|
| 1 | 95° C. | 1 | second | 15 | cycles |
| | 50° C. | 1 | second | | |
| 2 | 95° C. | 1 | min | 1 | cycle |
| 3 | 95° C. | 5 | seconds | 35 | cycles |
| | 55° C. | 10 | seconds | | |

Results showed that Salmonella in the milk sample was successfully detected.

Sensitivity of the subject method for detecting Salmonella in milk samples was then determined with the LOD (Limit Of Detection) test. Briefly, six samples were prepared by mixing 1800 μl milk sample not comprising any Salmonella with 200 μl Salmonella dilution of known concentration. The concentration of Salmonella in the samples was then determined by plating 100 μl of the sample on a Luria-Bertani (LB) agar plate, then the total number of viable colonies were counted. The samples were also amplified and analyzed using the method of the present disclosure, as described above. The results showed that the limit of detection (LOD) for Salmonella in the milk samples using the method of Example 27 is on average 2.9E+2 CFU/mL.

Thus, comparing to other methods and kits commercially available to analyze milk samples (e.g., real time PCR kits provided by Norgen Biotek Corp.), the method of the present disclosure may not require purification of milk samples, extraction of DNA, or incubation (e.g., for lysis) at elevated temperature (e.g., 100° C. or boiling temperature). The subject method takes less steps and costs much less time, yet, it may be used to successfully detect Salmonella in milk samples with higher sensitivity.

### Example 28: Amplification and Detection of Salmonella in Milk Samples with Enrichment Culturing

Amplification and detection experiments were performed on milk samples comprising unknown concentration of Salmonella, with culture enrichment. A milk sample comprising unknown amount of Salmonella was analyzed without sample purification, yet with enrichment culturing. Briefly, 2 mL milk sample was added into a 2 mL centrifuge tube. The tube was then centrifuged at 12,000 rpm for 2 min. The supernatant was removed. Then, 1 mL Tryptic Soy Broth (TSB) liquid medium was added into the tube and mixed thoroughly. The mixture was cultured at 37° C. for 1-3 h, with vigorous shaking. The cultured mixture was then centrifuged at 6,000 rpm for 2 min and the supernatant was removed. Then, 30 μl lysis buffer (100 mM NaOH, pH 12.5) was added and mixed thoroughly.

The mixture was incubated at room temperature for 10 minutes. Then, 3 μl of the mixture was added into a reaction vessel comprising 47 μl amplification reagents for performing PCR (e.g., DNA polymerase, primers, dNTPs, co-factors, suitable buffer, etc.).

The amplification reactions were performed according to the protocols described in Table 37 below.

TABLE 37
PCR Protocols Employed in Example 28

| | | | | | |
|---|---|---|---|---|---|
| 1 | 95° C. | 1 | second | 15 | cycles |
| | 50° C. | 1 | second | | |
| 2 | 95° C. | 1 | min | 1 | cycle |
| 3 | 95° C. | 5 | seconds | 45 | cycles |

| | | |
|---|---|---|
| 55° C. | 10 | seconds |

Results showed that Salmonella in the milk sample was successfully detected.

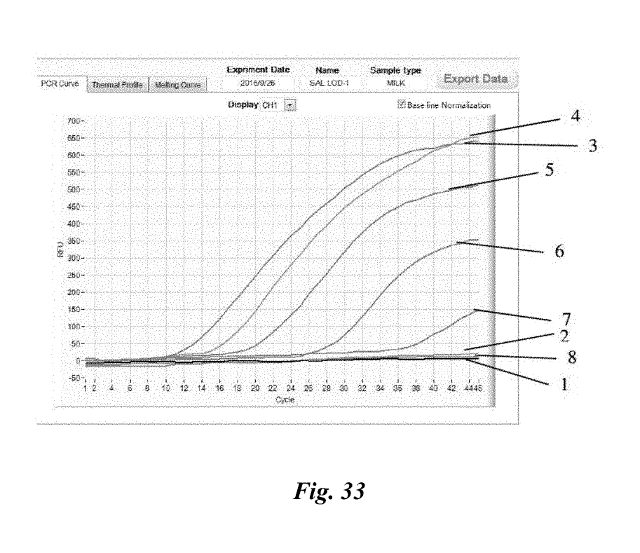

Sensitivity of the subject method for detecting Salmonella in milk samples was then determined with the LOD (Limit Of Detection) test. Briefly, six samples were prepared by mixing 1800 μl milk sample not comprising any Salmonella with 200 μl Salmonella dilution of known concentration. The concentration of Salmonella in the samples was then determined by plating 100 μl of the sample on a Luria-Bertani (LB) agar plate, then the total number of viable colonies were counted. The samples were also amplified and analyzed using the method of the present disclosure, as described above. The results showed that the limit of detection (LOD) for Salmonella in the milk samples using the method of Example 28 is on average 1 CFU/mL (see Table 38 below, also see FIG. 33).

TABLE 38
PCR Amplification Results of Example 28

| Sample | | | | |
|---|---|---|---|---|
| Well | Sample | | Channel | Ct value |
| 1 | PCR mix NTC | | FAM | — |
| 2 | Milk NTC | | FAM | — |
| 3 | 1.04E+4 | CFU/mL | FAM | 9.54 |
| 4 | 1.04E+3 | CFU/mL | FAM | 15.16 |
| 5 | 1.04E+2 | CFU/mL | FAM | 18.93 |
| 6 | 1.04E+1 | CFU/mL | FAM | 26.69 |
| 7 | 1.04 | CFU/mL | FAM | 36.08 |
| 8 | 1.04E−1 | CFU/mL | FAM | — |

Thus, comparing to other methods and kits commercially available to analyze milk samples (e.g., PCR kits provided by the 3M company), the method of the present disclosure may not require lengthened (e.g., 8-24 hrs) (pre)enrichment culturing, presumptive biochemical identification, extraction of DNA, or incubation (e.g., for lysis) at elevated temperature (e.g., 100° C. or boiling temperature). The subject method takes less steps and costs much less time, yet, it may be used to successfully detect Salmonella in milk samples with comparable or higher sensitivity.

### Example 29: Amplification and Detection of Norovirus GI and GII in Stool Samples

Amplification and detection experiments were performed on stool samples comprising Norovirus GI (50 copies/μl) and GII (20 copies/μl). The experiments were conducted using a Norovirus GI/GII Nucleic Acid Detection Kit (Coyotebio Inc.).

The kit comprises the following components:

(1) NoV GI/GII reaction mix, comprising Norovirus GI/GII primers, a fluorescent probe, internal reference primers and probe, dNTPs, and buffer;

(2) Enzyme mix, comprising a DNA polymerase and a reverse transcriptase;

(3) Nov GI/GII positive control, comprising a pseudovirus having GI/GII target sequences;

(4) Internal reference, comprising a plasmid comprising the target sequence of the internal reference;

(5) Negative control, comprising DNase & RNase free water; and

(6) Lysis buffer, comprising NaOH.

For each experiment, stool samples were suspended in 0.5 mL PBS by vortexing. The suspension was transferred into a 1.5 mL centrifuge tube and centrifuged at 13,000 rpm for 5 min. 10 μl to 50 μl of the supernatant was transferred into a fresh 1.5 mL centrifuge tube and heated to 95° C. for 5 min Subsequently, the supernatant was mixed with an equal volume of the lysis buffer and the mixture was vortexed for 15 seconds. 10 μl lysate was taken for performing PCR.

Each PCR amplification was conducted by adding 10 μl lysate, positive control, or negative control to 44.8 μl NoV GI/GII reaction mix, 4.2 μl enzyme mix, and 1 μl internal reference and the resultant mixture was subject to the protocols described in Table 39 below.

TABLE 39
PCR Protocols Employed in Example 29

| | | | | Cycle | |
|---|---|---|---|---|---|
| Pro. | Tem. | Time | | number | |
| 1 | 42° C. | 5 | min | 1 | cycle |
| 2 | 95° C. | 5 | seconds | 15 | cycles |
| | 45° C. | 5 | seconds | | |
| 3 | 95° C. | 1 | min | 1 | cycle |
| 4 | 95° C. | 5 | seconds | 40 | cycles |

| | |
|---|---|
| 55° C. | 30 seconds |
| | (fluorescent |
| | detection) |

Detection was conducted on a quantitative PCR system (e.g., Coyotebio Mini8 Plus). Fluorescence signal was read on the FAM channel

As can be seen from the results obtained from 20 samples of Norovirus GI and 20 samples of Norovirus GII as shown in Tables 40 and 41, Norovirus GI/GII can be successfully detected using the method of the present disclosure without sample purification. The LOD can be as low as 50 copies/μl (GI) and 20 copies/μl (GII) and consistency is high among samples.

TABLE 40
PCR Amplification Results for Norovirus GI

| Sample | | | |
|---|---|---|---|
| Well | Concentration | Template | Ct (FAM) |
| 1 | 50 copies/μl | 5 μl | 23.09 |
| 2 | 50 copies/μl | 5 μl | 24.96 |
| 3 | 50 copies/μl | 5 μl | 24.61 |
| 4 | 50 copies/μl | 5 μl | 24.11 |
| 5 | 50 copies/μl | 5 μl | 25.32 |
| 6 | 50 copies/μl | 5 μl | 24.39 |
| 7 | 50 copies/μl | 5 μl | 25.08 |
| 8 | 50 copies/μl | 5 μl | 26.51 |
| 9 | 50 copies/μl | 5 μl | 24.94 |
| 10 | 50 copies/μl | 5 μl | 27.53 |
| 11 | 50 copies/μl | 5 μl | 24.33 |
| 12 | 50 copies/μl | 5 μl | 24.04 |
| 13 | 50 copies/μl | 5 μl | 25.65 |
| 14 | 50 copies/μl | 5 μl | 24.72 |
| 15 | 50 copies/μl | 5 μl | 24.88 |
| 16 | 50 copies/μl | 5 μl | 24.33 |
| 17 | 50 copies/μl | 5 μl | 26.94 |
| 18 | 50 copies/μl | 5 μl | 26.14 |
| 19 | 50 copies/μl | 5 μl | 25.91 |
| 20 | 50 copies/μl | 5 μl | 25.54 |

TABLE 41
PCR Amplification Results for Norovirus GII

| Sample | | | |
|---|---|---|---|
| Well | Concentration | Template | Ct (FAM) |
| 1 | 20 copies/μl | 5 μl | 23.47 |
| 2 | 20 copies/μl | 5 μl | 24.72 |
| 3 | 20 copies/μl | 5 μl | 23.57 |
| 4 | 20 copies/μl | 5 μl | 24.81 |
| 5 | 20 copies/μl | 5 μl | 24.23 |
| 6 | 20 copies/μl | 5 μl | 24.71 |
| 7 | 20 copies/μl | 5 μl | 23.62 |
| 8 | 20 copies/μl | 5 μl | 24.78 |
| 9 | 20 copies/μl | 5 μl | 24.2 |
| 10 | 20 copies/μl | 5 μl | 22.91 |
| 11 | 20 copies/μl | 5 μl | 22.61 |
| 12 | 20 copies/μl | 5 μl | 24.67 |
| 13 | 20 copies/μl | 5 μl | 22.85 |
| 14 | 20 copies/μl | 5 μl | 23.18 |
| 15 | 20 copies/μl | 5 μl | 24.02 |
| 16 | 20 copies/μl | 5 μl | 23.31 |
| 17 | 20 copies/μl | 5 μl | 24.07 |
| 18 | 20 copies/μl | 5 μl | 24.60 |
| 19 | 20 copies/μl | 5 μl | 24.23 |
| 20 | 20 copies/μl | 5 μl | 25.20 |

Thus, comparing to other methods and kits commercially available to analyze stool samples (e.g., real time PCR kits provided by Shanghai ZJ Bio-Tech Co., Ltd. or provided by DAAN Gene Co., Ltd.), the method of the present disclosure may not require purification of stool sample, extraction of DNA, or incubation (e.g., for lysis) at elevated temperature (e.g., 100° C. or boiling temperature). The subject method takes less steps and costs much less time, yet, it may be used to successfully detect norovirus GI/GII in stool samples with higher sensitivity and consistency.

### Example 30: Amplification and Detection of Enterovirus in Stool Samples

Amplification and detection experiments were performed on stool samples comprising enterovirus 71 (38 copies/μl).

For each experiment, 0.1 g stool samples were suspended in 900 μl suspension buffer by vortexing in a 1.5 mL centrifuge tube, followed by centrifugation at 13,000 rpm for 10 min 100 μl of the supernatant was transferred into a fresh 1.5 mL centrifuge tube and heated to 70° C. for 2 min Subsequently, 10 μl supernatant was mixed with 1 μl lysis buffer and the mixture was vortexed for 5 seconds and was taken for performing PCR.

Each PCR amplification was conducted by adding 11 μl lysate to 45 μl reagents and the resultant mixture was subject to the protocols described in Table 42 below.

TABLE 42
PCR Protocols Employed in Example 30

| | | | | | |
|---|---|---|---|---|---|
| 1 | 95° C. | 5 | seconds | 15 | cycles |
| | 45° C. | 5 | seconds | | |
| 2 | 95° C. | 1 | min | 1 | cycle |
| 3 | 95° C. | 5 | seconds | 40 | cycles |

| | |
|---|---|
| 55° C. | 30 seconds |
| | (fluorescence |
| | detection) |

Detection was conducted on a quantitative PCR system (e.g., Coyotebio Mini8 Plus). Fluorescence signal was read on the FAM channel

As can be seen from the results obtained from 20 samples of Enterovirus 71 as shown in Table 43, enterovirus can be successfully detected using the method of the present disclosure without sample purification. The LOD can be as low as 38 copies/μl and consistency is high among samples.

TABLE 43
PCR Amplification Results for Enterovirus 71

| Sample | | |
|---|---|---|
| Well | Concentration | Ct (FAM) |
| 1 | 38 copies/μl | 19.17 |
| 2 | 38 copies/μl | 19.57 |
| 3 | 38 copies/μl | 20.17 |
| 4 | 38 copies/μl | 19.86 |
| 5 | 38 copies/μl | 19.46 |
| 6 | 38 copies/μl | 19.95 |
| 7 | 38 copies/μl | 20.00 |
| 8 | 38 copies/μl | 19.66 |
| 9 | 38 copies/μl | 17.68 |
| 10 | 38 copies/μl | 17.75 |
| 11 | 38 copies/μl | 18.34 |
| 12 | 38 copies/μl | 17.49 |
| 13 | 38 copies/μl | 18.06 |
| 14 | 38 copies/μl | 17.72 |
| 15 | 38 copies/μl | 17.83 |
| 16 | 38 copies/μl | 18.06 |
| 17 | 38 copies/μl | 21.96 |
| 18 | 38 copies/μl | 20.87 |
| 19 | 38 copies/μl | 20.70 |
| 20 | 38 copies/μl | 21.75 |
| 21 | Positive control | 19.43 |

Thus, comparing to other methods and kits commercially available to analyze stool samples (e.g., real time PCR kits provided by Shanghai ZJ Bio-Tech Co., Ltd. or provided by DAAN Gene Co., Ltd.), the method of the present disclosure may not require purification of stool sample, extraction of DNA, or incubation (e.g., for lysis) at elevated temperature (e.g., 100° C. or boiling temperature). The subject method takes less steps and costs much less time, yet, it may be used to successfully detect enterovirus in stool samples with higher sensitivity and consistency.

### Example 31: Amplification and Detection of Salmonella in Stool Samples

Amplification and detection experiments were performed on stool samples comprising Salmonella at 2.6×10⁶ CFU/g, 2.6×10⁵ CFU/g, 2.6×10⁴ CFU/g, 2.6×10³ CFU/g, and 2.6×10² CFU/g. Five separate experiments were conducted after cultivation for proliferation of bacteria.

For each experiment, 1.0 g stool sample was transferred into 10 mL SBG medium in 50 mL tube for cultivation. All cultures were vigorously shaken at 200 rpm, 37° C. for 4 hours, before samples were taken.

For each culture, 100 μl culture were taken and heated at 95° C. for 10 minutes, follow by addition of 10 μl lysis buffer. Subsequently, the lysate was centrifuged at 12,000 rpm for 3 minutes. 10 μl supernatant was taken as the template for subsequent amplification.

Amplification was conducted following the procedure as described in Table 44 using a primer set consisting of a forward primer and a reverse primer, as well as a sequence-specific fluorescent oligonucleotide probe, disclosed herein.

TABLE 44
PCR Protocols Employed in Example 31

| | | | | Cycle | |
|---|---|---|---|---|---|
| Pro. | Tem. | Time | | number | |
| 1 | 42° C. | 5 | min | 1 | cycle |
| 2 | 95° C. | 1 | second | 15 | cycles |
| | 50° C. | 1 | second | | |
| 3 | 95° C. | 1 | min | 1 | cycle |
| 4 | 95° C. | 5 | second | 35 | cycles |

| | |
|---|---|
| 55° C. | 30 seconds |
| | (fluorescent |
| | detection) |

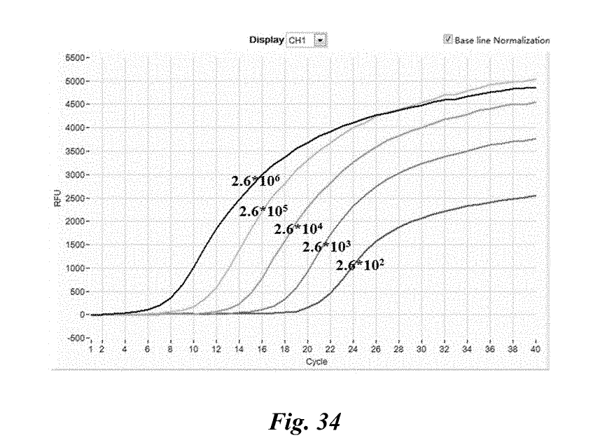

As can be seen from the results as shown in Table 45 and FIG. 34, Salmonella can be successfully detected using the method of the present disclosure without sample purification. The LOD can be as low as 2.6*10² CFU/g.

TABLE 45
PCR Amplification Results for Example 31

| | Initial Concentration (CFU/g) | | | | |
|---|---|---|---|---|---|
| | 2.6*10² | 2.6*10³ | 2.6*10⁴ | 2.6*10⁵ | 2.6*10⁶ |
| CT | 19.96 | 17.05 | 13.52 | 10.05 | 6.93 |

Thus, comparing to other methods and kits commercially available to analyze stool samples (e.g., real time PCR kits provided by Shanghai ZJ Bio-Tech Co., Ltd. or provided by DAAN Gene Co., Ltd.), the method of the present disclosure may not require purification of stool sample, extraction of DNA, or incubation (e.g., for lysis) at elevated temperature (e.g., 100° C. or boiling temperature). The subject method takes less steps and costs much less time, yet, it may be used to successfully detect Salmonella in stool samples with higher sensitivity.

### Example 32: Comparison Between Genome DNA and mRNA Amplification and Detection of Salmonella in Culture Samples

Sensitivities of amplification and detection of Salmonella using genome DNA and mRNA as target nucleic acids were compared by using Salmonella culture serial dilutions.

A Salmonella strain was taken from a −80° C. freezer for inoculating into liquid LB media. The strain was cultivated at 37° C., 200 rpm overnight. The culture was washed with 0.9% NaCl and centrifuged at 13,000 rpm for 3 min twice. 1:10 Serial dilutions of Salmonella was made in 0.9% NaCl. The initial concentration of Salmonella was 10⁶ CFU/μl. Dilution-3 (1,000 CFU/μl), Dilution-4 (100 CFU/μl), Dilution-5 (10 CFU/μl), and Dilution-6 (1 CFU/μl) were used for both genome DNA and mRNA amplification.

Sample preparation was conducted as described elsewhere herein. Reaction mixture without Salmonella lysate was used as the negative control.

For ttr DNA detection, a primer set consisting of a forward primer, a reverse primer, and a sequence-specific fluorescent oligonucleotide probe disclosed herein, were used.

For invA mRNA detection, a primer set consisting of a forward primer, a reverse primer, and a sequence-specific fluorescent oligonucleotide probe, disclosed herein, were used.

Amplification was conducted following the procedure as described in Table 46.

TABLE 46
PCR Protocols Employed in Example 32

| | | | | Cycle | |
|---|---|---|---|---|---|
| Pro. | Tem. | Time | | number | |
| 1 | 95° C. | 1 | second | 15 | cycles |
| | 50° C. | 1 | second | | |
| 2 | 95° C. | 1 | min | 1 | cycle |
| 3 | 95° C. | 5 | second | 40 | cycles |

| | |
|---|---|
| 55° C. | 30 seconds |
| | (fluorescent |
| | detection) |

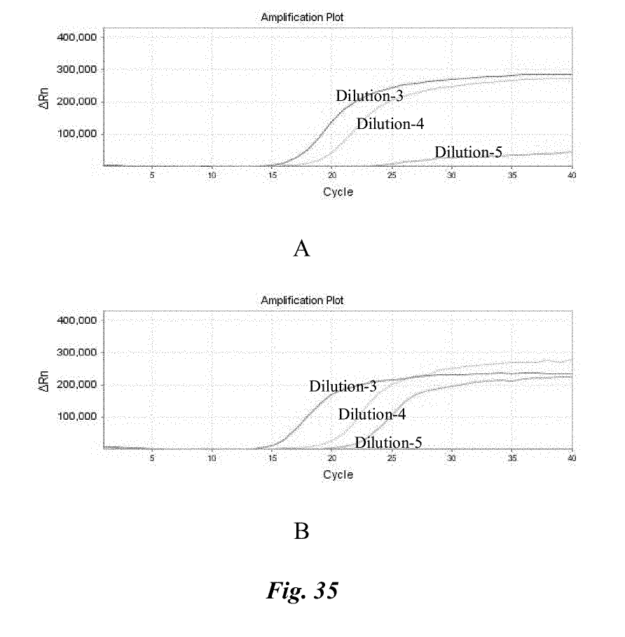

As can be seen from the results as shown in Table 47 and FIG. 35 (panel A for DNA detection and panel B for mRNA detection), Salmonella can be successfully detected using the method of the present disclosure by both DNA amplification and mRNA amplification. The LOD can be as low as 10 CFU/μl. Moreover, mRNA amplification resulted in higher sensitivity in all concentrations tested as compared to DNA amplification. This trend was more prominent towards the lower range of the concentrations.

TABLE 47
PCR Amplification Results for Example 32

| | Dilu- | Dilu- | Dilu- | Dilu- | Negative |
|---|---|---|---|---|---|
| | tion-3 | tion-4 | tion-5 | tion-6 | control |
| Salmonella | 1,000 | 100 | 10 | 1 | 0 |
| concentration | | | | | |
| (CFU/μl) | | | | | |
| DNA | 14.25 | 15.21 | 23.12 | — | — |
| detection (Ct) | | | | | |
| mRNA | 13.91 | 14.61 | 19.37 | — | — |
| detection (Ct) | | | | | |

### Example 33: Comparison Between Genome DNA and mRNA Amplification and Detection of Salmonella in Stool Samples

Sensitivities of amplification and detection of Salmonella using genome DNA and mRNA as target nucleic acids were compared by using Salmonella stool samples.

A stool sample was taken by dipping a swab into the stool surface and turning the swab one round. The swab was sunk into 500 μl 0.9% NaCl and vortex thoroughly.

Sample preparation was conducted as described elsewhere herein. The initial lysate (with a concentration of Salmonella at about 1,000 CFU/μl) and two dilutions (10× and 100×, respectively) were used for subsequent amplification. Stool sample without Salmonella was used as the negative control.

For ttr DNA detection, a primer set consisting of a forward primer, and a reverse primer, and a sequence-specific fluorescent oligonucleotide probe, as disclosed herein were used.

For invA mRNA detection, a primer set consisting of a forward primer, a reverse primer, and a sequence-specific fluorescent oligonucleotide probe, as disclosed herein were used.

Amplification was conducted following the procedure as described in Table 48.

TABLE 48
PCR Protocols Employed in Example 33

| | | | | Cycle | |
|---|---|---|---|---|---|
| Pro. | Tem. | Time | | number | |
| 1 | 95° C. | 1 | second | 15 | cycles |
| | 50° C. | 1 | second | | |
| 2 | 95° C. | 1 | min | 1 | cycle |
| 3 | 95° C. | 5 | second | 40 | cycles |

| | |
|---|---|
| 55° C. | 30 seconds |
| | (fluorescent |
| | detection) |

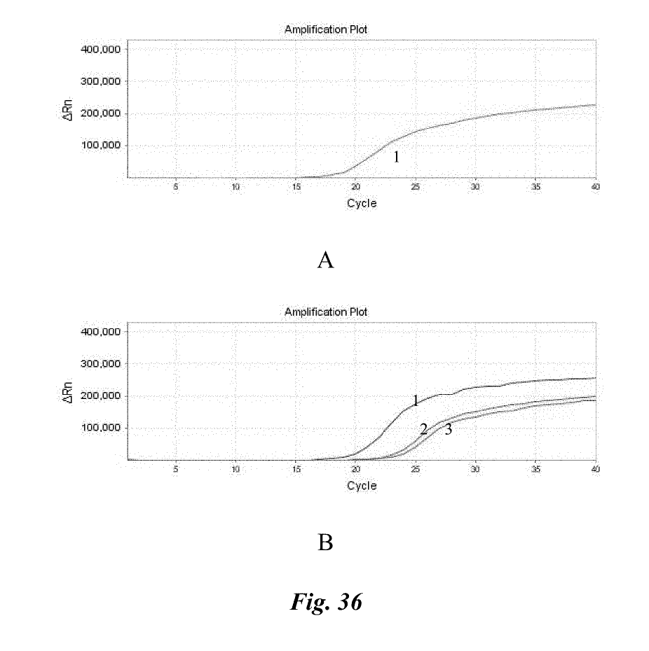

As can be seen from the results as shown in Table 49 and FIG. 36 (panel A for DNA detection and panel B for mRNA detection), Salmonella can be successfully detected using the method of the present disclosure by both DNA amplification and mRNA amplification without sample purification. The LOD can be as low as 10 CFU/μl for mRNA detection and 1000 CFU/μl for DNA detection. Moreover, mRNA amplification resulted in higher sensitivity tested as compared to DNA amplification. This trend was more prominent towards the lower range of the concentrations where Salmonella could be detected by mRNA amplification at 100 and 10 CFU/μl which were not detectable using DNA amplification.

TABLE 49
PCR Amplification Results for Example 33

Sample #

| | | | | Negative |
|---|---|---|---|---|
| | 1 | 2 | 3 | control |
| Salmonella | 1000 | 100 | 10 | 0 |
| concentration | | | | |
| (CFU/μl) | | | | |
| DNA | 16.99 | — | — | — |
| detection (Ct) | | | | |
| mRNA | 15.91 | 17.98 | 19.77 | — |
| detection (Ct) | | | | |

### Example 34: Combined Genome DNA and mRNA Amplification and Detection of Salmonella

Sensitivities of amplification and detection of Salmonella using combined genome DNA/mRNA and mRNA only as target nucleic acids were compared by using Salmonella culture serial dilutions.

Salmonella culture samples and serial dilutions were prepared as described elsewhere herein. Dilution 0 (1,000 CFU/μl), Dilution-1 (100 CFU/μl), Dilution-2 (10 CFU/μl), and Dilution-3 (1 CFU/μl) were used for both combined genome DNA/mRNA amplification and mRNA only amplification.

Sample preparation was conducted as described elsewhere herein. Reaction mixture without Salmonella lysate was used as the negative control.

For invA mRNA only detection, a primer set consisting of a forward primer, a reverse primer, and a sequence-specific fluorescent oligonucleotide probe, as disclosed herein were used.

For combined ttr DNA+invA mRNA detection, in addition to the aforesaid primer set and probe, another primer set consisting of a forward primer, a reverse primer, and another sequence-specific fluorescent oligonucleotide probe, as disclosed herein were also used.

Amplification was conducted following the procedure as described in Table 50.

TABLE 50
PCR Protocols Employed in Example 34

| | | | | Cycle | |
|---|---|---|---|---|---|
| Pro. | Tem. | Time | | number | |
| 1 | 95° C. | 1 | second | 15 | cycles |
| | 50° C. | 1 | second | | |
| 2 | 95° C. | 1 | min | 1 | cycle |
| 3 | 95° C. | 5 | second | 40 | cycles |

| | |
|---|---|
| 55° C. | 30 seconds |
| | (fluorescent |
| | detection) |

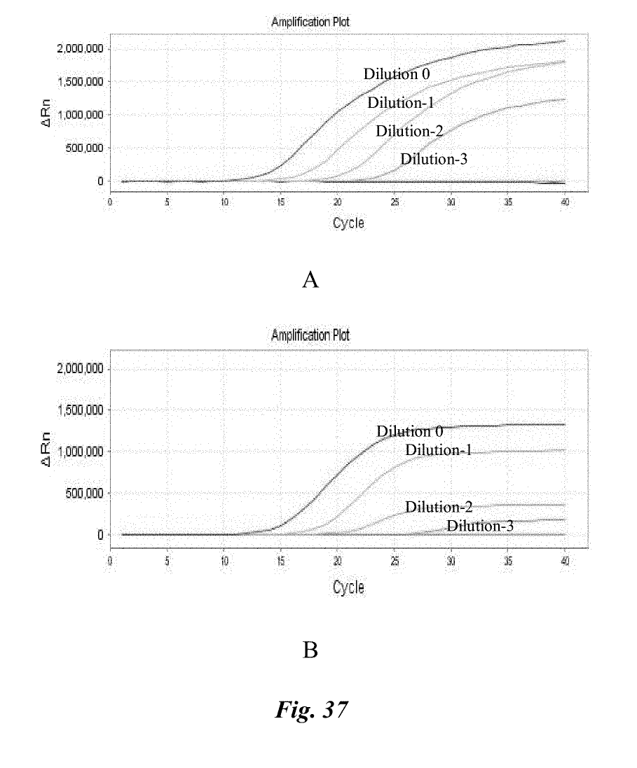

As can be seen from the results as shown in Table 51 and FIG. 37 (panel A for combined genome DNA/mRNA detection and panel B for mRNA only detection), Salmonella can be successfully detected using the method of the present disclosure by both combined genome DNA/mRNA amplification and mRNA amplification. The LOD can be as low as 1 CFU/μl. Moreover, Combined genome DNA/mRNA amplification resulted in even higher sensitivity in all concentrations tested as compared to mRNA only amplification. This trend was more prominent towards the lower range of the concentrations. These results show that it may be advantageous to target both genome DNA and mRNA in detecting Salmonella by amplification.

TABLE 51
PCR Amplification Results for Example 34

| | Dilu- | Dilu- | Dilu- | Dilu- | Negative |
|---|---|---|---|---|---|
| | tion 0 | tion-1 | tion-2 | tion-3 | control |
| Salmonella | 1,000 | 100 | 10 | 1 | 0 |
| concentration | | | | | |
| (CFU/μl) | | | | | |
| Combined | 11.09 | 14.45 | 18.24 | 22.59 | — |
| DNA/mRNA | | | | | |
| detection (Ct) | | | | | |
| mRNA | 11.95 | 15.31 | 20.11 | 26.07 | — |
| detection (Ct) | | | | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1.-249. (canceled)

250. A method for detecting a target nucleic acid molecule in a biological sample, comprising:
(a) mixing said biological sample with a lysis buffer to obtain a mixture;
(b) incubating said mixture at a temperature from about 15° C. to 70° C. for a period of time of no more than about 15 minutes;
(c) adding said mixture from (b) to a reaction vessel comprising reagents necessary for conducting nucleic acid amplification, said reagents comprising (i) a deoxyribonucleic acid (DNA) polymerase, and (ii) a primer set for said target nucleic acid molecule, to obtain a reaction mixture; and
(d) subjecting said reaction mixture in said reaction vessel to multiple cycles of a primer extension reaction to generate amplified product(s) that is indicative of a presence of said target nucleic acid molecule, each cycle comprising (i) incubating said reaction mixture at a denaturing temperature for a denaturing duration that is less than or equal to 60 seconds, followed by (ii) incubating said reaction mixture at an elongation temperature for an elongation duration that is less than or equal to 60 seconds, thereby amplifying said target nucleic acid molecule.

251. The method of claim 250, further comprising, prior to (a), suspending said biological sample in solution to obtain a homogenized preparation comprising said biological sample.

252. The method of claim 250, further comprising, prior to (a), subjecting said biological sample to centrifugation to yield a solution comprising said biological sample and a pellet.

253. The method of claim 250, further comprising, between (b) and (c), subjecting said mixture to centrifugation to yield a supernatant comprising said biological sample.

254. The method of claim 250, wherein said biological sample is obtained directly from a source thereof without pre-culturing, non-selective enrichment, selective enrichment, plating on differential medium, and/or presumptive biomedical identification.

255. The method of claim 250, wherein said biological sample is from a tissue of a subject, fluid of a subject, includes a soil sample or includes a food sample.

256. The method of claim 250, wherein said mixture is added to said reaction vessel in (c) without undergoing DNA or ribonucleic acid (RNA) extraction, without undergoing purification, or without undergoing DNA or RNA extraction.

257. The method of claim 250, wherein said biological sample is not treated with a detergent.

258. The method of claim 250, wherein in (a), a ratio of said biological sample to said lysis buffer is between about 1:1 (wt/vol) to about 1:10 (wt/vol).

259. The method of claim 250, wherein said reagents in (c) comprise a reporter agent that yields a detectable signal indicative of a presence of said amplified product(s).

260. The method of claim 250, wherein said denaturing duration or said elongation duration is less than or equal to about 30 seconds.

261. The method of claim 250, wherein said amplifying yields a detectable amount of said amplified product(s) indicative of a presence of said target nucleic acid molecule in said biological sample at a cycle threshold value (Ct) of less than 30.

262. The method of claim 250, further comprising detecting an amount of said amplified product(s).

263. The method of claim 250, further comprising outputting information indicative of an amount of said amplified product(s) to a recipient.

264. The method of claim 250, further comprising subjecting said target nucleic acid molecule to one or more denaturing conditions prior to (d).

265. A method for detecting a target nucleic acid molecule in a biological sample, comprising:
(a) mixing said biological sample with a lysis buffer to obtain a mixture;
(b) incubating said mixture at a temperature from about 15° C. to 70° C. for a period of time of no more than about 15 minutes;
(c) adding said mixture from (b) to a reaction vessel comprising reagents necessary for conducting nucleic acid amplification, said reagents comprising (i) a deoxyribonucleic acid (DNA) polymerase and optionally a reverse transcriptase, and (ii) a primer set for said target nucleic acid molecule, to obtain a reaction mixture; and
(d) subjecting said reaction mixture in said reaction vessel to a plurality of series of primer extension reactions to generate amplified product(s) that is indicative of a presence of said target nucleic acid molecule in said sample, each series comprising two or more cycles of (i) incubating said reaction mixture under a denaturing condition characterized by a denaturing temperature and a denaturing duration, followed by (ii) incubating said reaction mixture under an elongation condition characterized by an elongation temperature and an elongation duration, wherein an individual series differs from at least one other individual series of said plurality with respect to said denaturing condition and/or said elongation condition.

266. The method of claim 265, further comprising, prior to (a), suspending said biological sample in solution to obtain a homogenized preparation comprising said biological sample.

267. The method of claim 265, further comprising, prior to (a), subjecting said biological sample to centrifugation to yield a solution comprising said biological sample and a pellet.

268. The method of claim 265, further comprising, between (b) and (c), subjecting said mixture to centrifugation to yield a supernatant comprising said biological sample.

269. The method of claim 265, wherein said biological sample is obtained directly from a source thereof without pre-culturing, non-selective enrichment, selective enrichment, plating on differential medium, and/or presumptive biomedical identification.

270. The method of claim 265, wherein said biological sample is from a tissue of a subject, fluid of a subject, includes a soil sample or includes a food sample.

271. The method of claim 265, wherein said mixture is added to said reaction vessel in (c) without undergoing DNA or ribonucleic acid (RNA) extraction, without undergoing purification, or without undergoing DNA or RNA extraction.

272. The method of claim 265, wherein said biological sample is not treated with a detergent.

273. The method of claim 265, wherein in (a), a ratio of said biological sample to said lysis buffer is between about 1:1 (wt/vol) to about 1:10 (wt/vol).

274. The method of claim 265, wherein said reagents in (c) comprise a reporter agent that yields a detectable signal indicative of a presence of said amplified product(s).

275. The method of claim 265, wherein said denaturing duration or said elongation duration is less than or equal to about 30 seconds.

276. The method of claim 265, wherein said amplifying yields a detectable amount of said amplified product(s) indicative of a presence of said target nucleic acid molecule in said biological sample at a cycle threshold value (Ct) of less than 30.

277. The method of claim 265, further comprising detecting an amount of said amplified product(s).

278. The method of claim 265, further comprising outputting information indicative of an amount of said amplified product(s) to a recipient.

279. The method of claim 265, further comprising subjecting said target nucleic acid molecule to one or more denaturing conditions prior to (d).


SEQUENCE LISTING (9 sequences)

SEQ ID NO: 1 - DNA, 22 bp, Artificial Sequence
  Description of Artificial Sequence Synthetic primer
tgctcgtttacgacctgaatta

SEQ ID NO: 2 - DNA, 20 bp, Artificial Sequence
  Description of Artificial Sequence Synthetic primer
acaccaatatcgccagtacg

SEQ ID NO: 3 - DNA, 26 bp, Artificial Sequence
  Description of Artificial Sequence Synthetic probe
tctggttgatttcctgatcgcactga

SEQ ID NO: 4 - DNA, 20 bp, Artificial Sequence
  Description of Artificial Sequence Synthetic primer
tcgtttacgacctgaattac

SEQ ID NO: 5 - DNA, 19 bp, Artificial Sequence
  Description of Artificial Sequence Synthetic primer
tagaacgaccccataaaca

SEQ ID NO: 6 - DNA, 23 bp, Artificial Sequence
  Description of Artificial Sequence Synthetic probe
ctggttgatttcctgatcgcact

SEQ ID NO: 7 - DNA, 23 bp, Artificial Sequence
  Description of Artificial Sequence Synthetic primer
ctcaccaggagattacaacatgg

SEQ ID NO: 8 - DNA, 23 bp, Artificial Sequence
  Description of Artificial Sequence Synthetic primer
agctcagaccaaaagtgaccatc

SEQ ID NO: 9 - DNA, 22 bp, Artificial Sequence
  Description of Artificial Sequence Synthetic probe
caccgacggcgagaccgacttt